SEQUENCE LISTING (20 sequences)

SEQ ID NO: 1 - DNA, 1348 bp, Desulfovibrio piger
tcacgggtcgaaggcgcggatggtgcgcccaacattgacggggttgttgatgtccgggacctgcacaccgatgagggcttatcttctggtccttcacacggatgtagtggaccagagaaccacggggcgcttcggtgcagccgtagccttcggcgctctgcgggatctcgaagggggtgaaggtctcgccgtcgggcttgacttccttcagccagccttccacagcattggcgatgagcagggcttcttcggcgcgggccacgtgacggcccatgatggagaagaccttgtcccagcccaggtcgcggatggtgcgggcttcgatgccgaacttttccttcagcatcttcacgcccacttcggacagcggcgcgttggccacccacatgcgggcggcggggcccacttcgatgacttcgccgtcgtagcggggagccttgacgaaggagtaggcgtccttcttgtccaggttggggttggtctcggcgccggcagcacccttgccggtggtggcgtcgtcgtaccaggcgtacttgaggtcttcggtgatcttcttgggatcgaattccacatcacggccattgaggaagataccggccttgagcaggtgggtcttgccgtcgtcggtcatggggaacactcgaattccacatcacggccattgaggaagataccggccttgagcaggtgggtcttgccgtcgtcggtcatggggaacacgcaggtatttttcgacgatgaactggcgcaccttcttgaagcgggccgcgtattccagcagggcttccttggtgggcatttcagttgcaccgccaggcacgatgccctgcacatggggcatgcggccgccgaagagggcgaccatctcgtgggcgatgcggcgcacttccagggcttccaggtactggtccacgcccaccttgttggcttcggggggcaggcgcaggtcgggctgggcgaaacgcggcacgaagggcgcggtatcggggccggccacgaagtccagagcggccagatggtagaagtgcaggatgtgcgactgcaggtagttggcgcccaggatcaggttacgggtgatgcggccgttgccggtcaccttgatgttgaaggcgctttcctgcgccagggcggaagccatggcatgggaggtggggcacacgccgcagatgcgctgcacgatctgggtggagtcacgcgggtcgcgccctttcaaaatctgttcaaaaccgcggaacatcccaccggtgaggtgggcgtccacaaccttgccgttctccaccaccacttgggccttgagatggccctcgattctggtgacgggatcgatggcgatagtggccttggccat

SEQ ID NO: 2 - DNA, 951 bp, Desulfovibrio piger
ctagttgctgtagaaggggctctgtccgtcggggaacgagggctcggtgcagccgatgcagatggcgttggccacgcaccagttgacgccgttccatttacgttcaaacgagtcgcagtacgcaccggggcccttgcagcccaggtcgtagcggcagccgtccttctcggtgaaggtggcggagaacttgccttcgtcgtacttgcccagataggggcagttctcgtgcacgttgcgaccgtagaaaggcagcgggcggccttcgctgtccagcagggagaccacttcggccagaccggcttccaggcccttttccttgatcttctggagggccagggcgatggtgcccacgatccagtcaggctggggcgggcagccggggatgttgaccacgggggtcttgatgccggcctgcttgagcatggcaccggtacccatggcttcggtcacggaacccttggcagcggggatgccgccgtaggcagcgcaggtacccagggccagcacggcggcggcatcgggagccagctttttgaccagatcggccatggtgatctcggtgtggccctcttcggccaccacgcagtagcgaccatccttggccagcgggatggaaccttcgatggccaggaagaacttgcccttgaagttcttggcaatgttcagcatgtgggtgtaggcgtcgtggccttcaccgcccatgacggtaggatggtactcgaggctgatgatcttcagcagcacatcagcgatggaggggttgacgttgttcagcagcgacactgaacaaccggtacagccctgtccctgcagccagatgacaggggggcgttcgccagtcaacgtgccggccagggcttcgcgaaccgcaggatggaacatctgcgcaacgccgaaacccgccactgtgccggtgcacagtttgacgaaatcacgcctgttgagactcat

SEQ ID NO: 3 - DNA, 252 bp, Desulfovibrio piger
tcagaacaggggtttctgcccttcgatggcctcggccacctcgcgcagggctgccaggctctcctgggcctgctcaggggtgagactgtgcagggcgaagcccgcgtggacgatgacgtaatcaccgaccttgggcggctcgggcagcaggagcatggaagcggtaaggtaggtctggctttcgcccacacgcacgcgggccatgccctgttcctggagttcgaccacctgggccggaatggcaagacacat

SEQ ID NO: 4 - DNA, 741 bp, Desulfovibrio piger
ttaacaggtactgtcgggatcgttcccggcgtattggcgggacaggcgcaggatctccttgcggacctcatccagataggcggggaagcgttcctgcacgacgggagaaagctccatgctccaagaggtgtagtccgcgggctccatgcccatgacggtgaggttggggcgggagccgtggagcatctcggccatgcgcagggaatccagcaggtcgatgtcgtggatggagaggcgctgcttctcgttgtccaccagctggttctcggtcagatgatagaccgtgccgggtccggcatggccgtggacgatgtccaggatgagcacatgggcaaagcccttgaacagatagaagacatcctgggtgaaggttcccgcttccatgatggtgacgttttcgggccaggattccccggccagggtctgggcggcgtggacgccgacaccgtcgtcggtgaggagcaggttgccgatacccatgacaagcactttgtccatactatcctcggtacaggagttgcgcccgctgggggcgggatgtggtgtccgccgtgcctggtgccgcatgggcaggccatgcaggcgcggatgctccggcagggacggatcacggacagacgacagcatagcagcccggcggggtagccgcaaggggatggcggagaagggagggaagcccggcggatggaccatgccccagaaggccgccggaacacgggcacgaaaaaagggcggggtcac

SEQ ID NO: 5 - DNA, 1581 bp, Desulfovibrio piger
atgggcaaactgctggtatgctggctgctgctctgcggcctgagcctgctggccggctgccgggaagaggagaagggacaggccggtgcgccgcgcgaggagctggtctatgccagcaccaaggacatccgggacatcaatcctcacctgtacggcggcgagatggcagcccagggcatggtcttcgagcccctggtcatcaacacggcggaaggtgtccggccctggctggccgaaagctgggagatctcgcccgacggccgcgtctatacattccatctgcgccgtggtgtgaccttcagcgacgggacgcccttcgatgccgaggccgtccgcctgaacatggatgccatcatcgccaatcgcctgcgccacgcctggctggacatgatcaacgagatcgaacgccatgaagtcgtggacagccatacctggcgtctggtactcaagcacccctattatcccactctcatcgagctggggctgttgcggcctttccgtttcatctcgccgtcctgcttcatcgacggtcagacgcgcaacggcgtacgcggcctcgtgggcacgggcccgtggatactgaaggaacacaaggagaaccagtacgccctgttcacggccaatccctcttactggggagagaagccacgtttgcaggccgtgcgctggaaggtgatgcccgaccaccaggccatcctgctggccctgcacaagggggacgtggatctggtgttcggggccgatggcgacatgctcaacctggacaatttcgacgccatccgtcgtgaggggcgctatgcggcggccatcagccagcccatcgcctcgcgggccatactgctcaacgctcaccagccgttcacccgggagcgggacgtgcgcctggccctgcagtatgcggtggacagggaaggcatcgccgccaccatcctcaacggttccgagaccgtggcccccagcctgctggcctccacggtggcctattgcgacctgccgctggaggcgcgcggccatgacccggaaaaggcggcgcgtctgctggacggggcgggctggctcatggcggccgacggctggcgctacaaggacggacggagggccagcgtgcgcctgtactacaattcgcagaatgcgcaggagcgcagcatcggcgagtacatgcaggcggacctgaaaaagatcggggtggagatgaagatcgtgggcgaggagaagcaggcctttctggatcgccagaagagcggcgactttgaattgcagtattccctgtcctggggcacgccttacgacccgcagtcctatatttcctcgtggcgcatcccggcgcacggggactatcaggcccagctgggtctggagcgcaaggaatggctggacgcggccatcacccgtttgatgaccgagacggacgaggagcgccgcaaggccctgtatgcggagatcctcgggtatgtacatgatgagggcgtctacatcccgctgacctattcccgcaccaaggccgtccacgtgcgggagctcaagggcgtttcattcggcgtgtcgcagtacgagatccccttcgagaagatgtatttttga

SEQ ID NO: 6 - DNA, 825 bp, Desulfovibrio piger
atgagcggcagcgacatcttccttgccgtccgggatatctggaagacctatgccgtcccccacggagggggctcccggcccgtgctgcgggggctggatttctgtctggagcggggcgagatggcgggcctggtgggcgagagcggcagcggcaagagcacgctggcccgtctgctgctggggctggagcggcccgaccgtggtatcgtgctgctggaaggtcagcccttgcggcaatggcgggccgggggcgggaggctctccgtggtctttcaggactatgtgacctcggtcgatccgggctttacgctggcggaggccgtggacgagggtctggggccgggctgccgcctttcccgccgggagcggcgacgggaagtggacaggctgctggaacgcgtggggctttcgccttcattgtccggccgtctgccgcatgaactcagcggcggtcaggtgcagcgggcctgcatcgcgcgggcgctggccgcgcgaccgtcctttctggtgctggacgaggccgtgagctcgctggatgtcccggtacaggtgcagatactggaattgctgcgggacattcgcagcgacacgacctgccttttcatcacccatgatctccaggccgcgacgctggtctgtgacagtctgctggtcctggatcagggccgctgtgcggagcatctgccggtgtcgcagctgggtagcgcccgcagcccgcgcttgcggcggatgctggaaacagtcgtcccgttccgctccgcatgggaggggcgctgtgacgaagctgccgggacaggaagagcccccggggcgacgaaggtctga

SEQ ID NO: 7 - DNA, 156 bp, Desulfovibrio piger
ctaatgctcgtcgtcgtcaccgaagagccaggggcccttcttggggaacagcacggtgttccagtacaggacgtagacgggcagggtcacgaacatcatgacgtaggcccagttcttggtgtagaggaagtagtcgtaaaaggtatggaattccat

SEQ ID NO: 8 - DNA, 1020 bp, Desulfovibrio piger
ctagttggcgttctcggtcgcacgcggcttgtggcaggacgcgcaggacgtatcgaggggacgggcaacgtccatgccgtcatggcaacccatgcactgcagatgatacgcggccttcagcgtgggacgctcgggcacgcggggatcgatcttggcggagtggcagctgccgcacttgggcggagtagccgacagggggctgcgatggtggcaggtggcgcacagggtctcgggttcgctgtggaacgccttggccagattgtcgccttcaatgcgcttcatgagcgaagagacgtgacgacgatggttgaagaggttgggctcgaacttgtcggacaggctgccgatttccaccttgtaggggcaggcttcgggggccaggtagtccacgggcttgcgttcggcgatggtggcttcggccagggcgacgcggtcctcggcggacagcttgccggccacgcccagctggtactgttcgggggtcatcttggaggtgaccacgtggcagacgccacaccacttgtcgtcacgcttgggagtgacgatctcgtggcagccggcgcattcgcggcgttccttgtactgttcttcgtggcagctcacacagctcttgggcgtgacgccatccttgcgcggggcaatcttggtggcgtgcatggcgcgctcaaggttgatgaagttgccctcggctttgccttccacggtatgacaggtgctacaggccaccatgtcgcccgtgtgatggcaggccgtgcaatcgtcgatcttgtcgacgtgagcctgatggttgaatgtcaccggcgtcatgctggcgcccttgggattgggcttttcgctgaccggaaagagaaccgtggcattaggcatttccgcgtcggattcttccagacccaccgcaacagcgcggttggcttcgggaagcatcagacaggccacgccggccaaggcaatcaccgccgccaaaagcagtgctttaccgttcctcatgtgctcgtccttttgaaacagacttttccccat

SEQ ID NO: 9 - DNA, 1185 bp, Desulfovibrio piger
ctagtagccgcgtccgtacatgcggacgaacagcagggccgcggtgcggtacacgaagtgaccgagcttggaccacggcaggtaggcaaagagcatccacacaaagaccagatgcagatagtacaccacaaaggcgggctggatggcatcggccaggcggaagacctgggacaggatgcccgtcacggccaccagccagatgatgcccagcaggtaccagtcataccagctcgaaccctgatgcttggggttcagcttcagacggcgggcggtgagcatggccaggccacagaccagcatgatggcgccgaggttggccaggatcttcacagggtacagcagcggcatgggggtctcgatcttgatgagcgggatgatcttgccgccccagtggcccagagccacaacggccgtcacgaaggccaggatgacgaagctgtagaccaggatcatgtggccggccttgcggttgggcacataggtaccggtcttgggaccagcttcgcagtcgtcgaacttgcggtgggtgaccacttcgtcgatgagcacgtccagcaggtgccagtaccaggccttggtcttgccaagcacggtcaggcggccttcaggcttgaacatgccccagagcttgcgcacgcccttgtagaggatgaagcaggccccgaagaaggtgatcatgaagatggggtcgatggtgtagtcaccatagaacacctgaccgaacacgatgcggccgtcggcggcgcgggggaaccagttgccgtcattgaagccggcgcggatccaccagatgaagagccacagcagggccgggatggcgaagaggaagggcagaccggagggcttgctcatccacttgcccacgatggtgggctcggtcagatcgcggtagaccatgttgcgggcagcgcccatgacgtcggcgggacgggcaccacgggggcagaggtccgcgcagttgccgcagttgtggcagagccacaggtccacgtcggtggccagcttttccttgaggccccaggacgcccacaccatttccttgcgggggtagggagcgttttccggagccatggggcaggccacggagcaggtggcgcactgatagcattttttcaggctttcgccgcccgcttccatcagctcccgcgtaaaggtggaatccggcttgagaatattctgtgccat

SEQ ID NO: 10 - DNA, 1236 bp, Desulfovibrio piger
ctacctccctttcaccgtttgcaccgccttgagagcggcggccgtgccggactgcgcggtacgcatcacgtccagcggcatgcgggcgcaaccggcggcaaaaatgccagcctcttcaccgcccatgacgaaaccgtcttcgtccaggggcaggggcaggggcaggctgtcggtggccaggctgggctgcatgcccgtggccagcaccaccatgtcataatccagatgcagcttttcgccgcggatggcgtcttcaacggtcacggtgacgttgtcgtcagcggcctgggccacgtcggccaccttgcccttgatgaagcgcacattgggggcggccttcaccttgtccagaaccttcacatagcggcccggagcgcgcaggtcgatgtagaacaccgagatctgggtctcggggtactgctcgcagatgtactggcactgcttgagagtggccatacagcagatgtaggagcagtagttcaggtggttctggtcacgggaaccggcgcactgcacgaaagcgatgcgtttgggacgacggccgtcggtaggacggacgatcttgccgccggtggggccgaagggggaggccaggcgttcgagctgcatgttggtcacgcagttcttgaccttgccggcgcccaggttggctaggttggtgacgtcgtagggcttccagccggtggccaccacgatggcgcccacattgagttcgatctcgcgggcgggttcgttctggtccaggaacttctggccggcgacgcgggcggcgtcagccttgctcagggcgtcggtgtccagcacgaagcggttggggaacgcgaaaggcatggatttgtacagagccttgcgctgggacagcccgaagtcgaattcgttgggcacggtgccttccaggctggaagcgaggagggtgaagtccacgttgtgggcggccaccttgcggggcttgatgctcacggtggcggtgtagtcacccttacagccgaccagcttggtcacttcggccatggtgaagaatttgatgcgggggttcttcctgatgcgctggaactggatttccagaccgcaggaggggggacaaagtttggggaaatatttgttgagctgggccacacggccgcccaggtagggcgacttttccacgatatacacatcgtggcccagttccgccgcttcgatggcggcagtaaggcctgcaaagccaccgcccacgacgagaatggagttggacat

SEQ ID NO: 11 - DNA, 1167 bp, Desulfovibrio piger
ctagtcgctgaagtgaccggtttcgcgcttcacgcccagggcgatcttccagagcagggagagcaccagggcgcccacggcgtacacgcccagggccacgatgatctcacggccggtgggggcgtattcggtgacggtctcaaacatgttgggagtgaaaccaccgatgagcaggccgaggcccttgtcgatccaggaagcggcaaccagcagcaccagagccaggggcagcagggggccggtacgccactgcggcgggatcaggatgatcagggaagcggcggccatgaccacagcggcccacatccagttggtcacccaggaaacgtggccttcatgaccagcgaacaggaagaccaggggatgctgatgaccggggatgccactgtagaacgcggtgaagacttccagcaggtagaagaacacgttgatgcacatggcataggtgatgatggtcgtcagggtcttgatggcgtcgcggccgggatcgaagccggtgaggcggcgcacgatgaagatcagcagcagcaggatggcgggaccggagcagaacgccgaggacaggaagcgggcggccatgatggcggtcagccagtaatggcggccggggatgccggcgtacaggaaggcggtcacggtgtggatggagaaggcccacaggatcgagaggtagatcaggggcttgatccacttgggcggttccacgtcatggcgctcggcttccagggtgacccagccgatgacggcgttgagggtcaggtagccgatgagcacgatcatgtcatagaacatgaccgaattgggcgtgggatgcagcagcacgttcatcatgcgctggggctggcccaggtccaccacgatgaagagggcgcacatgaccacggccgagatggccatgaattcaccgaagatgatcatgcgcttgaacttcttgtagtggtggaagtaggcgggcagcaccagcatgacggcggaagcggccacacccacaagataggtgaactgcgagatgtagaggccccacgagacatcgcggctcatgccggtgatggagaggccttcctgcagctgctgcaggtagaccaggccgcaaccggcgatgactgcgagcaggaagagcagccacaggtagtaggtcttgggaccggtaagcaatttttcaagcat

SEQ ID NO: 12 - DNA, 1023 bp, Desulfovibrio piger
ttactcggcggccttggaagagggctgcttttccagcggcaggccggcttcggccatggcatcgcggtaaacgtcctcatattcggcataggtgaagtactgcttgggcgggttccaggggttcacgtggcgcacttcgcgggtgttggtgggaaggttgcgcgtggggctgaagaacacgccaggcatgtgcatgagcttggagaaggggaagtacagcagcagcacgctgacaaaggccagatgcatgaagaagagcgggctgatgccgtcgggggccacgggcgagaagtgcaccagacccatgatgaagaccttgacctgggcgatgtcggtcttatcgaagtagcgcatgcagataccgctcagaacgacgcccaggataaggaacagggggaagtagtcgttcatcagggagatgtaacgcagacgctggttgaacagacgacgggccagcaggaagagcacggccaccagcagcaggccgccggtccagaagaagcggggggcgccgatctgcatgatgccgtcgatgctttccatgaaggtcagccagccgggcaccggatccatgaagaaacggaagtggcggatgaagaccagcaggaagcagtaatggaacagcagggcgaagaaccacaaccacttggaggagtagtagatggtgcgggggcccttggtcacagggtcgaattcacgcacgtcggccgcggtgttgcggaacagcgaacggaagaagcagacttccaggaacatgcggacgaccacaccgaacttggtgttggggcagtcccacttttcatgtttgatgaagtccagcgacttttcctgacccccggtcgtggggatgctgaaaggcacgggcgatttggcccagtaaaccatgcgccagatgacgcccaggaagaaggtgactaccgccacatagggcaaaaccacgccgaacaggtacgacagaccaatggccgaacctgcccaggcaatggccccgatgagcgccaccaccagcaatgatgcgaacat

SEQ ID NO: 13 - DNA, 60 bp, Artificial Sequence
  SYNTHESIZED
ccatctcatccctgcgtgtctccgactcagnnnnnnnnnntggcagatmatgatymacgg

SEQ ID NO: 14 - DNA, 50 bp, Artificial Sequence
  SYNTHESIZED
cctatcccctgtgtgccttggcagtctcaggggccgtaaccgtccttgaa

SEQ ID NO: 15 - DNA, 20 bp, Artificial Sequence
  SYNTHESIZED
aaaggaagcaccggctaact

SEQ ID NO: 16 - DNA, 20 bp, Artificial Sequence
  SYNTHESIZED
cggattcaagtcgtgcagta

SEQ ID NO: 17 - DNA, 37 bp, Artificial Sequence
  SYNTHESIZED
gggaattccaattgagaccagccaggacagaaatgcc

SEQ ID NO: 18 - DNA, 34 bp, Artificial Sequence
  SYNTHESIZED
ctagtctagacggggtctgacgctcagtggaacg

SEQ ID NO: 19 - DNA, 34 bp, Artificial Sequence
  SYNTHESIZED
acgcggatccgggcgctcccgcaggggacagcgg

SEQ ID NO: 20 - DNA, 30 bp, Artificial Sequence
  SYNTHESIZED
gccatacctccacatggtttgttgtatcac

(19) United States
(12) Patent Application Publication
(10) Pub. No.: US 2016/0000837 A1
(43) Pub. Date: Jan. 7, 2016

(54) COMPOSITIONS AND METHODS TO ALTER GUT MICROBIAL FERMENTATION USING SULFATE-REDUCING BACTERIA

(71) Applicant: WASHINGTON UNIVERSITY, St. Louis, MO (US)

(72) Inventors: Federico E. Rey, St. Louis, MO (US); Mark Gonzalez, St. Louis, MO (US); Jeffrey I. Gordon, St. Louis, MO (US)

(21) Appl. No.: 14/768,394

(22) PCT Filed: Feb. 18, 2014
(86) PCT No.: PCT/US14/16883

§ 371 Date: Aug. 17, 2015

Related U.S. Application Data
(60) Provisional application No. 61/852,221, filed on Mar. 15, 2013.
(60) Provisional application No. 61/765,991, filed on Feb. 18, 2013.

(51) Int. Cl. A61K 35/741; A23L 1/29; A23L 1/30; A61K 31/737

(52) U.S. Cl. CPC A61K 35/741; A61K 31/737; A23L 1/293; A23L 1/3014; A61K 2035/115

(57) ABSTRACT

The present invention provides combinations and methods for changing the representation of at least one sulfate-reducing bacterial species in a subject's gut, thereby changing microbial fermentative activity in the gut in the subject.

### CROSS REFERENCE TO RELATED APPLICATIONS

This application claims the priority of PCT application No. PCT/US2014/016883, filed Feb. 18, 2014, which claims priority to U.S. provisional application No. 61/765,991, filed Feb. 18, 2013, and U.S. provisional application No. 61/852,221, filed Mar. 15, 2013, each of which is hereby incorporated by reference in its entirety.

### GOVERNMENTAL RIGHTS

This invention was made with government support under DK78669, DK70977, DK078669, and P30-AG028716 awarded by the National Institutes of Health. The government has certain rights in the invention.

### FIELD OF THE INVENTION

The present invention encompasses compositions and methods for changing the representation of sulfate-reducing bacteria in a subject's gut, thereby changing the microbial fermentative activity in the gut and changing adiposity in the subject.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821(f).

### BACKGROUND OF THE INVENTION

In the gut, fermentation is one digestive process that extracts energy from the available nutrient sources. Prior to the present invention, it was known in the art that clearing hydrogen gas generated by fermenting microbial communities through mechanisms that produce methane (methanogenesis), acetate (acetogenesis), or hydrogen sulfide (via sulfate reduction), affects energy extraction from available nutrient sources in the gut.

The hydrogen consuming bacteria in the gut that produce methane, acetate, and hydrogen sulfide, are referred to as methanogens, acetogens, and sulfate-reducing bacteria, respectively. Although features of the nutrient utilizing behavior of methanogens, acetogens and sulfate-reducing bacteria have been studied in vitro, little is known about the metabolic activities and requirements of these bacteria in vivo and how their metabolism impacts other microbes and the subject. Because little is known about the metabolic activities of these hydrogen consuming bacteria in vivo and, in particular, how their metabolism impacts the subject, it is not possible to predict the impact of existing or new food ingredients whose health effects or benefits are unclear. Thus, there is a need in the art for compositions and methods for altering the gut microbiota that will have defined effects on the representation of hydrogen consuming bacteria in the gut and clear impacts on the subject.

### SUMMARY OF THE INVENTION

The present invention encompasses a combination comprising a sulfated polysaccharide and an effective amount of at least one isolated Desulfovibrio species. The at least one isolated Desulfovibrio species comprises at least one nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). The isolated Desulfovibrio species may comprise any combination of 1, 2, 3, 4, 5, 6, 7, 8, 9, 10, 11 or 12 nucleic acids. The sulfated polysaccharide may be naturally occurring or synthetic, including but not limited to pentosan polysulfate, a fucoidan, a carrageenan, a sulfated glycosaminoglycan, or derivatives thereof. Optionally, the combination may further comprises an effective amount of at least one additional probiotic.

The present invention also encompasses a combination comprising a sulfated polysaccharide and an effective amount of at least one isolated SRB species selected from the group consisting of a D. piger and a bacterial species with at least one comparable in vivo fitness determinant to D. piger, wherein the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). The isolated SRB species may comprise any combination of 1, 2, 3, 4, 5, 6, 7, 8, 9, 10, 11 or 12 nucleic acids. The sulfated polysaccharide may be naturally occurring or synthetic, including but not limited to pentosan polysulfate, a fucoidan, a carrageenan, a sulfated glycosaminoglycan, or derivatives thereof. Optionally, the combination may further comprises an effective amount of at least one additional probiotic.

The present invention also encompasses a method for increasing microbial fermentative activity in the gut of a subject in need thereof. The method comprises administering a combination comprising a sulfated polysaccharide and an effective amount of at least one isolated Desulfovibrio species. The at least one isolated Desulfovibrio species comprises at least one nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). The isolated Desulfovibrio species may comprise any combination of 1, 2, 3, 4, 5, 6, 7, 8, 9, 10, 11 or 12 nucleic acids. The sulfated polysaccharide may be naturally occurring or synthetic, including but not limited to pentosan polysulfate, a fucoidan, a carrageenan, a sulfated glycosaminoglycan, or derivatives thereof. Optionally, the combination may further comprises an effective amount of at least one additional probiotic. When desired, an increase in microbial fermentative activity may be confirmed my determining in a sample obtained from the subject the amount of short chain fatty acids, hydrogen sulfide, abundance of the Desulfovibrio species, or combinations thereof, wherein an increased amount after administration of the combination relative to before administration confirms an increase in microbial fermentative activity.

The present invention also encompasses a method for increasing the nutritional value of a diet. The method comprises administering a combination comprising a sulfated polysaccharide and an effective amount of at least one isolated Desulfovibrio species. The at least one isolated Desulfovibrio species comprises comprises at least one nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In an aspect, the combination increases microbial fermentative activity in the gut of the subject, thereby increasing the nutritional value of the diet. The isolated Desulfovibrio species may comprise any combination of 1, 2, 3, 4, 5, 6, 7, 8, 9, 10, 11 or 12 nucleic acids. The sulfated polysaccharide may be naturally occurring or synthetic, including but not limited to pentosan polysulfate, a fucoidan, a carrageenan, a sulfated glycosaminoglycan, or derivatives thereof. Optionally, the combination may further comprises an effective amount of at least one additional probiotic. When desired, an increase in microbial fermentative activity may be confirmed my determining in a sample obtained from the subject the amount of short chain fatty acids, hydrogen sulfide, abundance of the Desulfovibrio species, or combinations thereof, wherein an increased amount after administration of the combination relative to before administration confirms an increase in microbial fermentative activity.

The present invention encompasses a method for increasing microbial fermentative activity in the gut of a subject in need thereof. The method comprises administering a combination comprising a sulfated polysaccharide and an effective amount of at least one isolated SRB species selected from the group consisting of a D. piger and a bacterial species with at least one comparable in vivo fitness determinant to D. piger, wherein the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). The isolated SRB species may comprise any combination of 1, 2, 3, 4, 5, 6, 7, 8, 9, 10, 11 or 12 nucleic acids. The sulfated polysaccharide may be naturally occurring or synthetic, including but not limited to pentosan polysulfate, a fucoidan, a carrageenan, a sulfated glycosaminoglycan, or derivatives thereof. Optionally, the combination may further comprises an effective amount of at least one additional probiotic. When desired, an increase in microbial fermentative activity may be confirmed my determining in a sample obtained from the subject the amount of short chain fatty acids, hydrogen sulfide, abundance of the SRB species, or combinations thereof, wherein an increased amount after administration of the combination relative to before administration confirms an increase in microbial fermentative activity.

The present invention also encompasses a method for increasing the nutritional value of a diet. The method comprises administering a combination comprising a sulfated polysaccharide and an effective amount of at least one isolated SRB species selected from the group consisting of a D. piger and a bacterial species with at least one comparable in vivo fitness determinant to D. piger, wherein the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In an aspect, the combination increases microbial fermentative activity in the gut of the subject, thereby increasing the nutritional value of the diet. The isolated SRB species may comprise any combination of 1, 2, 3, 4, 5, 6, 7, 8, 9, 10, 11 or 12 nucleic acids. The sulfated polysaccharide may be naturally occurring or synthetic, including but not limited to pentosan polysulfate, a fucoidan, a carrageenan, a sulfated glycosaminoglycan, or derivatives thereof. Optionally, the combination may further comprises an effective amount of at least one additional probiotic. When desired, an increase in microbial fermentative activity may be confirmed my determining in a sample obtained from the subject the amount of short chain fatty acids, hydrogen sulfide, abundance of the SRB species, or combinations thereof, wherein an increased amount after administration of the combination relative to before administration confirms an increase in microbial fermentative activity.

The present invention also encompasses a method for increasing the proportional representation of at least one SRB species in the gut of a subject. The method comprises administering a combination comprising a sulfated polysaccharide and an effective amount of at least one isolated SRB species selected from the group consisting of a D. piger and a bacterial species with at least one comparable in vivo fitness determinant to D. piger, wherein the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). The isolated SRB species may comprise any combination of 1, 2, 3, 4, 5, 6, 7, 8, 9, 10, 11 or 12 nucleic acids. The sulfated polysaccharide may be naturally occurring or synthetic, including but not limited to pentosan polysulfate, a fucoidan, a carrageenan, a sulfated glycosaminoglycan, or derivatives thereof. Optionally, the combination may further comprises an effective amount of at least one additional probiotic. When desired, an increase in the proportional representation of one or more SRB species may calculated by determining the abundance of one or more nucleic acid sequences encoding an enzyme involved in sulfate reduction or hydrogen consumption, including, but not limited to, DsrA, DsrB, DsrD, DsrJ, DsrK, DsrM, DsrO, DsrP, AprA, AprB, Sat, QmoA, QmoB, QmoC, HysA, HysB or a combination thereof.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1 A-C graphically depicts the sulfate-reducing bacteria in the fecal microbiota of healthy adult humans. The sulfate reductase alpha subunit (aprA) was amplified by PCR from fecal samples obtained from human subjects previously identified as SRB carriers (individual samples are identified on the y-axis; Hansen et al, 2011). Amplicons were subjected to multiplex pyrosequencing with a 454 FLX instrument using Titanium chemistry (see Methods for details). Sequences were analyzed using QIIME pipeline software tools. Reads were classified into OTUs on the basis of sequence similarity; we specified that species-level phylotypes share ≧94% identity over the sequenced region.

FIG. 2A-D depicts graphs and images showing the effects of host diet on a defined model human gut microbiota. (A) Relative abundance of bacterial species in the feces of mice fed a low fat/high plant polysaccharide diet (LF/HPP) or a diet high in fat and simple sugars (HF/HS). Abundance was defined by shotgun sequencing of fecal DNA (COPRO-Seq) 7 days after gavage with a consortium of 9 sequenced members of the human gut microbiota (n=4-5 animals/diet). Bacterial species that exhibited a significant difference in their abundance in the fecal microbiota of mice consuming one or the other diet are highlighted in red text in the figure legend (p<0.05, Student's t-test). Community structure remains stable on each diet until the time of sacrifice 14 d after colonization (see FIG. 26). (B) Selected results from microbial RNA-Seq analysis of the fecal meta-transcriptome. The heat map shows a subset of mRNAs encoding ECs whose expression was significantly different as a function of host diet (fold-difference ≦2 or >2; p<0.01, PPDE>0.95). The maximal relative expression across a row is red; the minimum is green (see legend at the bottom). Each column represents a different mouse in the indicated treatment group. Mean values ±S.E.M are plotted. (C and D) Targeted gas chromatography-mass spectrometry (GC-MS) analysis of hydrogen sulfide (C) and short chain fatty acids (SCFAs) (D) in cecal contents as a function of diet (n=4-5 animals/diet). Mean values ±S.E.M are plotted. *, p<0.05 based on Student's t-test. Comparison of two groups of mice fed the HF/HS diet and colonized with the 9-member community or another with the same community minus D. piger revealed that the presence of D. piger was associated with a statistically significant 1.8±0.3-fold higher level of H2S in cecal contents (n=5 mice/treatment group; p<0.05, two-tailed t-test; data not shown).

FIG. 3A-D depicts graphs and images presenting the INSeq analysis of D. piger fitness determinants in vitro and in vivo. (A) Graphical representation of the output:input ratio of individual transposon mutant genes, composed of ˜16,000 intragenic insertions across the D. piger GOR1 genome, after in vitro selection in a defined medium containing lactate, sulfate and all 20 amino acids. Mutants that show a significant drop in representation in the fecal microbiota (padj<0.05) and are present at output:input ratio <0.3 are highlighted in red. Those genes with no statistically significant change in abundance are highlighted in blue while those with no or low counts (mean<20 INSeq reads) are highlighted in green and excluded from analysis. For details on the genes that correspond to those in the first two categories and their known or predicted functions see Table S9 of Rey et al. PNAS 110: 13582-13587. (B) Venn diagram of the number of D. piger fitness determinants identified in the fecal microbiota of mice fed the LF/HPP or HF/HS diet that are present at output:input ratio <0.3 (padj<0.05) (n=4 mice/diet). (C) Ammonia assimilation genes that exhibit diet- and biogeography-dependent fitness effects based on INSeq analysis of mouse fecal pellets and cecal contents obtained 7 days after colonization with the D. piger mutant library (n=4 mice/group). Shown is the output:input ratio for each gene, with the D. piger gene annotation noted. The significance of the difference in representation of the indicated mutant strain in the output population compared to the input library in the fecal versus cecal microbiota: *padj<0.05; **padj<0.001 (negative binomial test from DESeq package; Anders and Huber, 2010). Significance of the difference observed in fecal samples obtained from mice on the LF/HPP versus HF/HS diets # padj<0.001. (D) Measurement of ammonia levels in fecal and cecal samples collected from mice colonized with the 9-member community containing D. piger fed the LF/HPP versus HF/HS diets. Mean values ±S.E.M. are plotted. * p<0.05 based on Student's t-test.

FIG. 4 depicts an illustration showing fitness determinants identified by INSeq in D. piger grown in vitro using lactate as the electron donor and sulfate as the electron acceptor. Growth of D. piger in a fully defined medium containing lactate as an electron donor and sulfate as electron acceptor occurs through the uptake and oxidation of lactate, which supplies electrons for sulfate reduction. This pathway generates a proton gradient that is used to generate energy via an F-type ATP synthase. Solid arrows represent enzyme reaction steps, while dashed arrows represent electron transfer steps (e-). Proteins and protein complexes involved in these reactions are noted, with those identified as statistically significant fitness determinants in red. Asterisks denote genes that had insufficient INSeq read counts for analysis in the input population (<20 reads; see Tables s5 and s9 of Rey et al PNAS 110: 13582-13587). LctP, lactate permease, DpigGOR1_{—}1075; Ldh, lactate dehydrogenase, DpigGOR1_{—}0371 and DpigGOR11628; Por, pyruvate-ferredoxin oxidoreductase, DpigGOR1_{—}1331; Pta, phosphate acetyltransferase, DpigGOR1_{—}1330; AckA, acetate kinase, DpigGOR1_{—}1329; Sat, sulfate adenylyltransferase, DpigGOR1_{—}0178; PpaC, pyrophosphatase, DpigGOR1_{—}2264; AprB, adenylsulfate reductase b subunit, DpigGOR1_{—}0794; AprA, adenylsulfate reductase a subunit, DpigGOR1_{—}0793; QmoA, quinone-interacting membrane-bound oxidoreductase flavin protein, DpigGOR1_{—}0792; QmoB, quinone-interacting membrane-bound oxidoreductase flavin protein, DpigGOR1_{—}0791; QmoC, quinone-interacting membrane-bound oxidoreductase membrane FeS protein, DpigGOR1_{—}0790; DsrA, dissimilatory sulfite reductase alpha subunit, DpigGOR1_{—}2316; DsrB, dissimilatory sulfite reductase beta subunit, DpigGOR1_{—}2317; DsrD, dissimilatory sulfite reductase D subunit, DpigGOR1_{—}2318; DsrMKJOP, DpigGOR1_{—}0174-DpigGOR1_{—}0170; ATP synthase, DpigGOR1_{—}0309-DpigGOR1_{—}0315.

FIG. 5 depicts graphs showing levels of wild-type D. piger versus the aggregate D. piger library of transposon mutants in the fecal microbiota of gnotobiotic mice harboring the 9-member model human gut community and fed the LF/HPP versus HF/HS diet. The relative abundance of the D. piger INSeq library was defined in fecal samples obtained from mice fed a low fat/high plant polysaccharide diet (LF/HPP) or a high fat/high simple sugar diet (HF/HS) using COPRO-Seq. Samples were taken 7 days after gavage with the library (n=4 mice/diet). Also shown is the relative abundance of wild-type (wt) D. piger from FIG. 2 (n=4-5 mice/diet). Note that there are no statistically significant differences between the levels of the aggregate INSeq library and wild-type D. piger in groups of mice consuming the same diet (Student's t-test). Mean values ±S.E.M are plotted.

FIG. 6A-B depicts graphs showing evidence for sulfate cross-feeding between B. thetaiotaomicron and D. piger. (A) In vitro test of sulfate cross-feeding. Plotted on the left y-axis is D. piger growth (OD600) in filter-sterilized conditioned medium harvested from B. thetaiotaomicron cultures of the sulfatase maturation mutant (Δbt0238) and isogenic wild-type (wt) strains grown in triplicate in minimal medium with chondroitin sulfate or fructose. The results of targeted GC-MS analysis of H₂S levels produced during D. piger growth in B. thetaiotaomicron-conditioned medium are plotted on the right y-axis. Mean values ±S.E.M. are shown (n=3/sample). (B) Quantitative PCR analysis of D. piger levels in mice co-colonized with either wild-type or Δbt0238 B. thetaiotaomicron. Mean values ±S.E.M. are plotted (n=3/sample). *, p<0.05 based on Student's t-test.

FIG. 7 depicts a graph showing the effects of different levels and types of sulfur-containing diet supplements on levels of D. piger. The relative abundance of D. piger was determined by shotgun sequencing of fecal DNA (COPRO-Seq). Six groups, each composed of two co-housed mice colonized with the 9-member model human gut microbiota were fed one of 13 diets, all based on the HF/HS diet (0.12% w/w SO4; see Table S2 of Rey et al. PNAS 110: 13582-13587 for diet composition). Each group of mice were started on the HF/HS diet and then given a sequence of four diets with differing sulfur content, each for a 7-day period. The sequence of presentation of the four diets was randomized so that that each diet was eventually fed to two different groups of co-housed animals. Mean values ±S.E.M are plotted. *, p<0.05 based on one-way ANOVA (Dunnett's Multiple Comparison Test). Abbreviations: SO4, sulfate; Cys, cysteine; Met, methionine; 503, sulfite; S203, thiosulfate; Chond. 504, chondroitin sulfate.

FIG. 8 presents an illustration summarizing the findings from Examples 1-9. B. thetaiotaomicron sulfatase activity liberates sulfate from sulfated mucins and produces H₂ during fermentation, providing D. piger with a source of sulfate and an electron source for its sulfate reduction pathway. This pathway yields H₂S, which can freely diffuse into enterocytes and inhibit mitochondrial acyl-CoA dehydrogenase (with resulting accumulation of acylcarnitines) and cytochrome c oxidase (cyto. c oxid.) (enzymes highlighted in red). Solid arrows represent enzyme reaction steps or movement of molecules, while dashed arrows represent electron transfer steps (e-) or numerous enzyme reactions. Abbreviations; Sat, sulfate adenylyltransferase encoded by DpigGOR1_{—}0178; PpaC, pyrophosphatase (DpigGOR1_{—}2264); AprB, adenylsulfate reductase b subunit (DpigGOR1_{—}0794); AprA, adenylsulfate reductase a subunit (DpigGOR1_{—}0793); QmoA, quinone-interacting membrane-bound oxidoreductase flavin protein (DpigGOR1_{—}0792); QmoB, quinone-interacting membrane-bound oxidoreductase flavin protein (DpigGOR1_{—}0791); QmoC, quinone-interacting membrane-bound oxidoreductase membrane FeS protein (DpigGOR1_{—}0790); DsrA, dissimilatory sulfite reductase alpha subunit (DpigGOR1_{—}2316); DsrB, dissimilatory sulfite reductase beta subunit (DpigGOR1_{—}2317); DsrD, dissimilatory sulfite reductase D subunit (DpigGOR1_{—}2318) as well as other components associated with the reductase (DsrMKJOP encoded by DpigGOR1_{—}0174-DpigGOR1_{—}0170); ATP synthase (DpigGOR1_{—}0309-DpigGOR1_{—}0315). IM, inner membrane, OM, outer membrane.

FIG. 9A-E graphically depicts data showing the impact of D. piger on the artificial human gut microbiota and host. (A) Bacterial species from the eight-member artificial community that showed significant changes in abundance in the fecal microbiota when D. piger was present versus absent. Mice (n=19-20/treatment group; three independent experiments) were fed the HF/HS diet supplemented with 3% chondroitin sulfate; *P<0.05 (Mann-Whitney test). (B) GC-MS and UPLC-MS (*) analysis of cecal contents from the mice described in A. Metabolites that were significantly changed when D. piger was present in mice consuming the HF/HS diet supplemented with chondroitin sulfate are listed. Normalized MS peak areas were mean centered and unit variance scaled. Scores ±SEM are plotted (P<0.05, Student t test). (C) Microbial RNA-Seq analysis of the fecal metatranscriptome in response to colonization with D. piger. The heat map shows selected ECs encoded by mRNA that were differentially represented between the two conditions [fold-change <-2 or >2; P<0.01, posterior probability of differential expression (PPDE)>0.95]. Each column represents a different mouse in the indicated treatment group sampled 14 d after colonization. The maximal relative expression across a row is red; the minimum is green. (D and E) Targeted GC-MS analysis of cecal short chain fatty acid and H2S levels [n=19-20 mice; mean values ±SEM are plotted; *P<0.05 (Student t test)].

### DETAILED DESCRIPTION OF THE INVENTION

The compositions and methods of the invention are based on the discovery that (i) Desulfovibrio piger, a sulfate-reducing bacteria, can invade an established model human microbiota; (ii) the presence of D. piger in the gut of a subject affects hydrogen consumption in the gut, such that net effect of increased D. piger colonization in a subject's gut is increased hydrogen consumption; (iii) the presence of D. piger in the gut of a subject affects overall gut microbial fermentative activity, such that the net effect of increased D. piger colonization in a subject's gut is increased fermentative activity and a corresponding increase in the conversion of polysaccharides to end-products of fermentation; and (iv) the abundance and metabolic properties of D. piger (and, therefore, gut microbial fermentative activity in a subject) can be manipulated by dietary supplementation.

Accordingly, the present invention provides compositions and methods for changing the representation of sulfate-reducing bacterial (SRB) species in a subject's gut. Non-limiting examples of SRB genera found in the gut include Desulfovibrio, Desulfotomaculum, Desulfobulbus, and Desulfobacter. The present invention contemplates a change in any SRB species capable of colonizing the gut of a subject, though bacterial species belonging to the genus Desulfovibrio are particularly preferred. Non-limiting examples of Desulfovibrio spp. found in the gut include D. piger, D. intestinalis, D. vulgaris, D. fairfieldensis and D. desulfuricans. For a brief overview of taxonomic overview of SRB species, see Muyzer G and Stams A J Nature Review Microbiology 2010; 6:441-454, hereby incorporated by reference in its entirety. In each aspect of the invention describe herein, a change in the representation of sulfate-reducing bacteria may be either an increase or a decrease.

The phrase “representation of SRB species”, as used herein, refers to the diversity of all the SRB species in the gut of a subject, the absolute representation of a single SRB species in the gut of a subject, or the proportional representation of a single SRB species in the gut of a subject. In an aspect, the present invention provides methods for changing the diversity of the SRB species in the gut of a subject. For example, if a SRB species not present in a subject's gut is administered to the subject and colonizes the subject's gut, then the diversity of the SRB species in the subject's gut increases. In another aspect, the present invention provides methods for changing the absolute representation of a single SRB species. A change in the absolute representation of a single SRB species may or may not change the absolute representation of all SRB species in the gut. In another aspect, the present invention provides methods for changing the proportional representation of one or more SRB species relative to the total gut microbiota. For example, the amount of 1, 2, 3, 4, 5, 6, 7, 8, 9, 10, 11, 12, 13, 14, 15, 16, 17, 18, 19, or 20 or more SRB species may be changed relative to the total gut microbiota. In another aspect, the present invention provides methods for changing the proportional representation of one of more SRB species relative to all SRB species present in the gut. For example, the amount of 1, 2, 3, 4, 5, 6, 7, 8, 9, 10, 11, 12, 13, 14, 15, 16, 17, 18, 19, or 20 or more SRB species may be changed relative to the total SRB community in the gut of a subject. In another aspect, the present invention provides methods for changing the proportional representation of one of more SRB species relative to a specific SRB genus present in the gut. For example, the amount of 1, 2, 3, 4, 5, 6, 7, 8, 9, 10, 11, 12, 13, 14, 15, 16, 17, 18, 19, or 20 or more SRB species may be changed relative to the total of all species in a particular SRB genus in the gut of a subject.

Changing the representation of SRB species in a subject's gut can change microbial fermentative activity in the gut. In an aspect, the present invention provides a method for increasing microbial fermentative activity in the gut of a subject by increasing the representation of at least one SRB species. In another aspect, the present invention provides a method for decreasing microbial fermentative activity in the gut of a subject by decreasing the representation of at least one SRB species.

The term “microbial fermentative activity”, as used herein, refers to the biotransformation of foods comprised of polysaccharides to the end products of fermentation by microbes. An increase in microbial fermentative activity in the gut of a subject may result in greater energy extraction from available nutrient sources or, stated another way, may increase the caloric value of food. Ultimately, this may lead to an increase in the subject's body mass. Conversely, a decrease in microbial fermentative activity in the gut of a subject may result in less energy extraction from available nutrient sources or, stated another way, may decrease the caloric value of food. Ultimately, this may lead to a decrease in the subject's body mass.

The phrase “efficiency of microbial fermentation in the gut”, as used herein, refers to the efficiency of energy extraction from available nutrient sources by fermenting bacteria in the gut of a subject.

The terms “gut microbial community” and “gut microbiota”, as used herein, are interchangeable and refer to microbes that have colonized and inhabit the gastrointestinal tract of a subject. A subject's gut microbiota may be naturally acquired or artificially established. Means by which a subject naturally acquires its gut microbiota are well known. Such examples may include, but are not limited to, exposure during birth, environmental exposure, consumption of foods, and coprophagy. Means by which a subject's gut microbiota may be artificially established are also well known. For example, artificially established gut microbial communities can be established in gnotobiotic animals by inoculating an animal with a defined or undefined consortium of microbes. Typically, a naturally acquired gut microbiota is comprised of both culturable and unculturable components. An artificially acquired gut microbiota may be similarly comprised of both culturable and unculturable components, or may consist of only culturable components. The phrase “culturable components” refers to the bacteria comprising the gut microbiota that may be cultured in vitro using techniques known in the art. Culture collections of gut microbial communities are described in detail in PCT/US2012/028600, incorporated herein in its entirety by reference. A subject's existing gut microbiota may also be modified or manipulated, for example, by administering one or more isolated bacterial species, dietary supplements, or changing the subject's diet.

The terms “colonize” and “invade”, as used herein, are interchangeable and refer to establishment, without regard to the presence or absence of an existing microbial community. For example, bacteria may colonize the intestinal tract of both a gnotobiotic animal and an animal with an existing gut microbiota. In the context of animals with an existing gut microbiota, the colonizing bacteria function within the existing microbiota. Colonization may refer to a change in the absolute or proportional representation of the microbe.

The term “subject,” as used herein, refers to a monogastric animal. Contemplated within the scope of the invention are all nonruminant animals, including hind-gut fermentators. Non-limiting examples of monogastric organisms may include felines, canines, horses, humans, non-human primates, pigs (including swine), poultry, rabbits, and rodents. In further embodiments, “subject” may refer to fish. Preferred subjects include, but are not limited to, those with a decreased proportional representation of SRB species in their gut, more preferably a decreased proportional representation of Desulfovibrio species, more preferably a decreased proportional representation of D. piger. Methods of identifying suitable subjects are described below in Section III.

The phrase “dietary supplement”, as used herein, refers to a nutrient added to a diet that promotes the colonization, invasion, growth, and/or metabolic activity of a gut microbe or an isolated bacterial species administered to a subject. The term “supplement’, as used herein, is shorthand for “dietary supplement”. Also included in the term “supplement” are specific foods, that when added to the diet provides an increased amount of a nutrient. For example, seaweed is a specific food that could be added to a diet to increase sulfated polysaccharides. A dietary supplement may also refer to a “food additive” or “feed additive”.

The term “nutrient”, as used herein, refers to prebiotics, vitamins, carbohydrates, fiber, fatty acids, amino acids, sulfates, minerals, antioxidants and other food ingredients. Also included in the definition are enzyme cofactors. Suitable vitamins may include, but are not limited to: vitamin B1, vitamin B2, vitamin B3, vitamin B5, vitamin B6, vitamin B9, vitamin B12, lipoic acid, vitamin A, biotin, vitamin K, vitamin C, vitamin D, and vitamin E. Suitable minerals may include, but are not limited to compounds containing: iron, copper, magnesium, manganese, molybdenum, nickel, and zinc. Suitable enzyme cofactors may include, but are not limited to: adenosine triphosphate (ATP), S-adenosyl methionine (SAM), coenzyme B, coenzyme M, coenzyme Q, glutathione, heme, methanofuran, and nucleotide sugars. Suitable carbohydrates include, but are not limited to, pectins, hemicellulose and beta-glucans, cellulose-related compounds, starches/fructans/alpha-glucans, host-derived glycans, monosaccharides, carrageenan, porphyran, alpha-mannan, and alginic acid. Carbohydrates may be described as plant-derived (e.g. pectins, hemicellulose and beta-glucans, cellulose-related compounds, starches/fructans/alpha-glucans, monosaccharides, carrageenan, porphyran, and alginic acid), host-derived (i.e. produced by the host (i.e. the subject) that is harboring the bacterium, such as host-derived glucans), or others, such as alpha-mannan. Pectins may include, but are not limited to, arabinan, arabinoglalactan, pectic galactan, polygalacturonic acid, rhamnogalacturonan I, and rhamnogalacturonan II. Hemicelluloses and beta-glucans may include, but are not limited to, xylan or xylan derivatives (non-limiting examples include arabinoxylan, water soluble xylan, glucuronoxylan, arabinoglucuronoxylan), xyloglucan, glucomannan, galactomannan, beta-glucan, lichenin, and laminarin. Cellulose-related compounds may include, but are not limited to, cellobiose and cellulose. Starches, fructans and alpha-glucans may include, but are not limited to, amylopectin, pullulan, dextran, inulin and levan. Host-derived glucans include neutral mucin O-glycans, chondroitin sulfate, hyaluronic acid, heparin, keratan sulfate, and glycogen. Monosaccharides may include, but are not limited to, arabinose, fructose, fucose, galactose, galacturonic acid, glucose, glucuronic acid, glucosamine, mannose, N-acetylgalactosamine, N-acetylglucosamine, N-acetylneuraminic acid, rhamnose, ribose, and xylose. Suitable forms of sulfate may include, but are not limited to, sulfated polysaccharides, calcium sulfate, copper sulfate, ferrous sulfate, magnesium sulfate, manganese sulfate, sodium sulfate, vanadyl sulfate, and zinc sulfate. Suitable fibers (including both soluble and insoluble fibers) may include, but are not limited to, arabinoxylans, cellulose, resistant starch, resistant dextrins, inulin, lignin, chitins, pectins, beta-glucans and oligosaccharides. Suitable lipids may include, but are not limited to, fatty acids, glycerolipids, glycerophospholipids, sphingolipids, sterol lipids, prenol lipids, saccharolipids and polyketides. Suitable amino acids may include, but are not limited to glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, and arginine. Additional non-limiting examples of nutrients may include Thiamin, Riboflavin, Niacin, Folate, Pantothenic acid, Calcium, Phosphorus, Magnesium, Manganese, Iron, Zinc, Copper, Selenium, Sodium, Potassium, betacarotene, retinol, alphatocopherol, betatocopherol, gammatocopherol, deltatocopherol, alphatoctrienol, betatoctrienol, gammatocotrienol, deltatocotrienol, apo-8-carotenal, trans-lycopene, cis-lycopene, trans-beta-carotene, and cis-beta-carotene, caffeine.

The term “sulfated polysaccharide” refers to a polysaccharide conjugated to a sulfate and includes both naturally occurring sulfated polysaccharides and sulfated polysaccharides prepared by chemical sulfonation of a polysaccharide or any other method known in the art. Non-limiting examples of sulfated polysaccharides may include dextran sulfate, pentosan polysulfate, fucoidan, carrageenans (i.e. the family of linear polysaccharides extracted from red seaweeds), sulfated glycosaminoglycans, and derivatives thereof.

The term “prebiotic,” as used herein, refers to a food ingredient that is utilized by a gut microbe. Non-limiting examples of prebiotics may include dietary fibers, lipids (including fatty acids), proteins/peptides and free amino acids, carbohydrates, and combinations thereof (e.g., glycoproteins, glycolipids, lipidated proteins, etc.).

The term “probiotic”, as used herein, refers to at least one live isolated microorganism that, when administered to a subject in an effective amount, confers a health benefit on the subject.

The term “health benefit”, as used herein, refers to a change in the representation of sulfate-reducing bacteria in the gut of the subject, a change in microbial fermentative activity in the gut of the subject, a change in body mass of the subject, a change in the caloric value of one or more foods consumed by the subject, or a combination thereof. The terms “health benefit” and “beneficial effect” may be used interchangeably.

The term “effective amount”, as used herein, means an amount of a substance (e.g. a combination of the invention, or component comprising a combination), that leads to measurable and beneficial effect(s) for the subject administered the substance, i.e., significant efficacy. The effective amount or dose of the substance administered according to this discovery will be determined by the circumstances surrounding the case, including the substance administered, the route of administration, the status of the symptoms being treated, the benefit desired, among other considerations.

The phrase “fitness determinant”, as used herein, refers to a chromosomal nucleic acid sequence that contributes to the fitness of a bacterium, such that loss of expression from this locus decreases the overall fitness of the bacterium. Criticality for fitness may or may not be context dependent. For example, core fitness determinants are required regardless of the experimental condition being studied (e.g. in vivo vs. in vitro, a first diet vs. a second diet). Non-limiting examples of core fitness determinants may include a chromosomal nucleic acid sequence encoding a nucleic acid product involved in core functions such as cell division, DNA replication and protein translation. Alternatively, by comparing fitness determinants required for two different conditions (e.g. in vivo and in vitro, a first diet with one or more nutrients and a second diet lacking one or more nutrients), it can be determined which fitness determinants are context dependent. For example, by comparing in vivo fitness determinants (i.e. fitness determinants for growth in vivo) to in vitro fitness determinants (i.e. fitness determinants for growth in vitro), a skilled artisan can identify in vivo-specific fitness determinants (i.e. fitness determinants unique to in vivo growth). As another example, by comparing fitness determinants identified for a first diet containing one or more nutrients to fitness determinants for a second diet lacking the one or more nutrients, a skilled artisan can identify diet-specific fitness determinants. Particularly useful fitness determinants may be in vivo, diet-specific fitness determinants, where the diet is known to support invasion.

A “nucleic acid product”, as used herein, refers to a nucleic acid derived from a chromosomal nucleic acid sequence. For example, a nucleic acid product may be a mRNA, tRNA, rRNA, or cDNA. Also included in the definition of “nucleic acid product” are amino acid sequences encoded by a chromosomal nucleic acid. Therefore, “nucleic acid product” also refers to proteins and peptides encoded by a chromosomal nucleic acid.

The phrase “diet-responsive”, as used herein, refers to differential expression of a nucleic acid product by a bacterial species between two diets. Stated another way, a nucleic acid product that is preferentially utilized by an isolated bacterial species when growing on a first diet as compared to a second diet is a diet-responsive nucleic acid product. In the context of in vitro growth, “diet” refers to the growth medium. In the context of in vivo growth in the gut of a subject, “diet” refers to the food or chow consumed by the subject.

Other aspects of the compositions and methods of the invention are described in further detail below.

### I. Combinations Comprising at Least One Isolated Sulfate-Reducing Bacterial (SRB) Species and at Least One Sulfated Polysaccharide

The present invention provides combinations comprising at least one isolated SRB species and at least one sulfated polysaccharide. When administered to a subject, combinations of the invention may increase the representation of the at least one isolated SRB species and/or increase microbial fermentative activity in the subject's gut.

### A. At Least One Isolated SRB Species

In an aspect, the present invention provides combinations comprising at least one isolated SRB capable of colonizing the gut of a subject. SRB species are obligate anaerobic bacteria that use sulfate as a terminal electron acceptor, undergoing dissimilatory sulfate reduction. Sulfate-reducing activity is not limited to a particular phylogenetic group. Moreover, there is considerable variation in SRB carriage among subjects. SRB capable of colonizing the gut of a subject are known in the art, having been identified in the fecal microbiota obtained from healthy and unhealthy subjects. In some embodiments, an isolated SRB species suitable for use in this invention may be a member of the genus Desulfovibrio, Desulfomonas, Desulfotomaculum, Desulfobulbus, or Desulfobacter. In preferred embodiments, a combination of the invention comprises an isolated Desulfovibrio species. Non-limiting examples of suitable Desulfovibrio species include D. piger, D. intestinalis, D. vulgaris, D. fairfieldensis and D. desulfuricans. In an exemplary embodiment, a combination of the invention comprises at least one isolated SRB species selected from the group consisting of D. piger and an SRB species with at least one comparable in vivo fitness determinant to D. piger. In another exemplary embodiment, a combination of the invention comprises at least one isolated SRB species selected from the group consisting of D. piger and a Desulfovibrio species with at least one comparable in vivo fitness determinant to D. piger.

An isolated SRB species with at least one comparable in vivo fitness determinant to D. piger may have at least one, at least two, at least three, at least four, at least five, at least six, at least seven, at least eight, at least nine, at least ten or more comparable in vivo fitness determinants to D. piger. Alternatively, an SRB species with at least one comparable in vivo fitness determinant to D. piger may have at least 15, at least 20, at least 25, at least 30, at least 35, at least 40, at least 45, or at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200 or more comparable in vivo fitness determinants to D. piger. Methods of identifying in vivo fitness determinants are known in the art and include, but are not limited to, a genome-wide transposon mutagenesis method known as Insertion Sequencing (INSeq). INSeq is further detailed in Goodman A L et al. Cell Host Microbe (2009) 6(3):279-289, hereby incorporated by reference in its entirety. Further details regarding INSeq and, specifically, D. piger in vivo fitness determinants may also be found in the Examples.

In some embodiments, a D. piger in vivo fitness determinant is a core fitness determinant. Non-limiting examples of D. piger core fitness determinants may be found in Table 1. In other embodiments, a D. piger in vivo fitness determinant is an in vivo-specific determinant. Non-limiting examples may be found in Table 3. In other embodiments, a D. piger in vivo fitness determinant is a diet-responsive determinant. Non-limiting examples may be found in Table 2. In preferred embodiments, a D. piger in vivo fitness determinant is involved in hydrogen consumption. Non-limiting examples D. piger in vivo fitness determinants involved in hydrogen consumption include a predicted periplasmic [NiFeSe] hydrogenase complex (e.g. DpigGOR11496 and/or DpigGOR11497) important in other Desulfovibrio species for growth in H₂; hydrogenase maturation genes (e.g. DpigGOR10739 and/or DpigGOR1740); and/or a predicted transport system for nickel, which functions as an important cofactor for the hydrogenase (e.g. DpigGOR11393 and/or DpigGOR11398). In other preferred embodiments, a D. piger in vivo fitness determinant is involved in sulfate reduction. Non-limiting examples D. piger in vivo fitness determinants involved in sulfate reduction include a high molecular weight cytochrome complex, Hmc (e.g. DpigGOR10741 and/or DpigGOR10744); the QmoABC complex (e.g. DpigGOR10790 and/or DpigGOR10792) which are two electron transport systems required for sulfate reduction in other species (Dolla et al., 2000; Keon et al., 1997; Zane et al., 2010); and/or components of sulfite reductase (e.g. DpigGOR10170 and/or DpigGOR10174).

The phrase “comparable in vivo fitness determinant to D. piger” refers to a fitness determinant in an SRB species other than D. piger that contributes the same or a comparable function as a D. piger in vivo fitness determinant. In some embodiments, a comparable in vivo fitness determinant to D. piger may not have significant homology to a D. piger in vivo fitness determinant at the sequence level but performs the same function. For example, two proteins may be very distantly related and have diverged so extensively that sequence comparison cannot reliably detect their similarity; however, these two proteins may perform the same function (e.g. enzymatic activity, signaling, etc.). Methods for identifying proteins that lack sequence homology but share the same function are known in the art. Non-limiting examples include structural alignment, motif finding, comparison of Enzyme Commission (EC) number, or comparison of KEGG Orthology identifiers. For example, a comparable in vivo fitness determinant can have the same EC number or belong to the same KEGG group but not have at least 80% identity at the sequence level. In other embodiments, a comparable in vivo fitness determinant to D. piger may have significant homology to a D. piger in vivo fitness determinant at the amino acid or nucleic acid level. The comparable in vivo fitness determinant to D. piger may be at least 80, 85, 90, or 95% homologous to a biomolecule a D. piger in vivo fitness determinant. In one embodiment, a comparable in vivo fitness determinant to D. piger may be at least 80, 81, 82, 83, 84, 85, 86, 87, 88, or 89% homologous to a D. piger in vivo fitness determinant. In another embodiment, a comparable in vivo fitness determinant to D. piger may be at least 90, 91, 92, 93, 94, 95, 96, 97, 98, 99, or 100% homologous to a D. piger in vivo fitness determinant.

In another embodiment, a comparable in vivo fitness determinant to D. piger may be at least 80, 81, 82, 83, 84, 85, 86, 87, 88, or 89% homologous to a gene derived from Table 1. In another embodiment, a comparable in vivo fitness determinant to D. piger may be at least 90, 91, 92, 93, 94, 95, 96, 97, 98, 99, or 100% homologous to a gene derived from Table 1. In another embodiment, a comparable in vivo fitness determinant to D. piger may be at least 80, 81, 82, 83, 84, 85, 86, 87, 88, or 89% homologous to a gene derived from Table 3. In another embodiment, a comparable in vivo fitness determinant to D. piger may be at least 90, 91, 92, 93, 94, 95, 96, 97, 98, 99, or 100% homologous to a gene derived from Table 3. In another embodiment, a comparable in vivo fitness determinant to D. piger may be at least 80, 81, 82, 83, 84, 85, 86, 87, 88, or 89% homologous to a gene derived from Table 2. In another embodiment, a comparable in vivo fitness determinant to D. piger may be at least 90, 91, 92, 93, 94, 95, 96, 97, 98, 99, or 100% homologous to a gene derived from Table 2.

In some preferred embodiments, a comparable in vivo fitness determinant to D. piger may be at least 80, 81, 82, 83, 84, 85, 86, 87, 88, or 89% homologous to a fitness determinant selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In other preferred embodiments, a comparable in vivo fitness determinant to D. piger may be at least 90, 91, 92, 93, 94, 95, 96, 97, 98, 99, or 100% homologous to a fitness determinant selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

In determining whether a comparable in vivo fitness determinant to D. piger has significant homology or shares a certain percentage of sequence identity with a sequence of the invention, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details.

A SRB species may be present in a combination of the invention in from at least about 0.5% to 100% relative to the total weight (expressed as dry weight). For example, a SRB species may be present in a combination of the invention in about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0%, about 19.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 30.5%, about 31.0%, about 31.5%, about 32.0%, about 32.5%, about 33.0%, about 33.5%, about 34.0%, about 34.5%, about 35.0%, about 35.5%, about 36.0%, about 36.5%, about 37.0%, about 37.5%, about 38.0%, about 38.5%, about 39.0%, about 39.5%, about 40.0%, about 40.5%, about 41.0%, about 41.5%, about 42.0%, about 42.5%, about 43.0%, about 43.5%, about 44.0%, about 44.5%, about 45.0%, about 45.5%, about 46.0%, about 46.5%, about 47.0%, about 47.5%, about 48.0%, about 48.5%, about 49.0%, about 49.5%, about 50.0%, about 50.5%, about 51.0%, about 51.5%, about 52.0%, about 52.5%, about 53.0%, about 53.5%, about 54.0%, about 54.5%, about 55.0%, about 55.5%, about 56.0%, about 56.5%, about 57.0%, about 57.5%, about 58.0%, about 58.5%, about 59.0%, about 59.5%, about 60.0%, about 60.5%, about 61.0%, about 61.5%, about 62.0%, about 62.5%, about 63.0%, about 63.5%, about 64.0%, about 64.5%, about 65.0%, about 65.5%, about 66.0%, about 66.5%, about 67.0%, about 67.5%, about 68.0%, about 68.5%, about 69.0%, about 69.5%, about 70.0%, about 70.5%, about 71.0%, about 71.5%, about 72.0%, about 72.5%, about 73.0%, about 73.5%, about 74.0%, about 74.5%, about 75.0%, about 75.5%, about 76.0%, about 76.5%, about 77.0%, about 77.5%, about 78.0%, about 78.5%, about 79.0%, about 79.5%, about 80.0%, about 80.5%, about 81.0%, about 81.5%, about 82.0%, about 82.5%, about 83.0%, about 83.5%, about 84.0%, about 84.5%, about 85.0%, about 85.5%, about 86.0%, about 86.5%, about 87.0%, about 87.5%, about 88.0%, about 88.5%, about 89.0%, about 89.5%, about 90.0%, about 90.5%, about 91.0%, about 91.5%, about 92.0%, about 92.5%, about 93.0%, about 93.5%, about 94.0%, about 94.5%, about 95.0%, about 95.5%, about 96.0%, about 96.5%, about 97.0%, about 97.5%, about 98.0%, about 98.5%, about 99.0%, about 99.5%, or about 100% relative to the total weight (expressed as dry weight). Alternatively, a combination of the invention may comprise from about 20¹ to about 20⁹ cfu/g of live microorganisms per gram of the combination, or equivalent doses calculated for inactivated or dead microorganisms or for microorganism fractions or for produced metabolites.

### B. At Least One Sulfated Polysaccharide

In another aspect, a combination of the invention comprises at least one sulfated polysaccharide. For example, a combination of the invention may comprise at least 1, at least 2, at least 3, at least 4, or at least 5, at least 6, at least 7, at least 8, at least 9, at least 10 or more sulfated polysaccharides (each in an equal or varying amount). A sulfated polysaccharide may or may not be naturally occurring. In some embodiments, a sulfated polysaccharide is selected from the group consisting of a dextran sulfate, a pentosan polysulfate, a fucoidan, a carrageenan, a sulfated glycosaminoglycan, and derivatives thereof. Non-limiting examples of carageenans may include kappa carrageenan, iota carrageenan, and lambda carrageenan. Non-limiting examples of sulfated glycosaminoglycans may include dermatan sulfate, keratan sulfate, heparan sulfate, and chondroitin sulfate.

The amount of sulfated polysaccharide in the combination can and will vary. A sulfated polysaccharide may be present in a combination of the invention in from at least about 0.5% to 100% relative to the total weight (expressed as dry weight). For example, a sulfated polysaccharide of the invention may be present in a combination of the invention in about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0%, about 19.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 30.5%, about 31.0%, about 31.5%, about 32.0%, about 32.5%, about 33.0%, about 33.5%, about 34.0%, about 34.5%, about 35.0%, about 35.5%, about 36.0%, about 36.5%, about 37.0%, about 37.5%, about 38.0%, about 38.5%, about 39.0%, about 39.5%, about 40.0%, about 40.5%, about 41.0%, about 41.5%, about 42.0%, about 42.5%, about 43.0%, about 43.5%, about 44.0%, about 44.5%, about 45.0%, about 45.5%, about 46.0%, about 46.5%, about 47.0%, about 47.5%, about 48.0%, about 48.5%, about 49.0%, about 49.5%, about 50.0%, about 50.5%, about 51.0%, about 51.5%, about 52.0%, about 52.5%, about 53.0%, about 53.5%, about 54.0%, about 54.5%, about 55.0%, about 55.5%, about 56.0%, about 56.5%, about 57.0%, about 57.5%, about 58.0%, about 58.5%, about 59.0%, about 59.5%, about 60.0%, about 60.5%, about 61.0%, about 61.5%, about 62.0%, about 62.5%, about 63.0%, about 63.5%, about 64.0%, about 64.5%, about 65.0%, about 65.5%, about 66.0%, about 66.5%, about 67.0%, about 67.5%, about 68.0%, about 68.5%, about 69.0%, about 69.5%, about 70.0%, about 70.5%, about 71.0%, about 71.5%, about 72.0%, about 72.5%, about 73.0%, about 73.5%, about 74.0%, about 74.5%, about 75.0%, about 75.5%, about 76.0%, about 76.5%, about 77.0%, about 77.5%, about 78.0%, about 78.5%, about 79.0%, about 79.5%, about 80.0%, about 80.5%, about 81.0%, about 81.5%, about 82.0%, about 82.5%, about 83.0%, about 83.5%, about 84.0%, about 84.5%, about 85.0%, about 85.5%, about 86.0%, about 86.5%, about 87.0%, about 87.5%, about 88.0%, about 88.5%, about 89.0%, about 89.5%, about 90.0%, about 90.5%, about 91.0%, about 91.5%, about 92.0%, about 92.5%, about 93.0%, about 93.5%, about 94.0%, about 94.5%, about 95.0%, about 95.5%, about 96.0%, about 96.5%, about 97.0%, about 97.5%, about 98.0%, about 98.5%, about 99.0%, about 99.5%, or about 100% relative to the total weight (expressed as dry weight).

A subject's diet, when supplemented with a combination of the invention, may contain up to about 5% sulfated polysaccharide. For example, a subject's total diet may contain at least about 5%, about 4.5%, about 4%, about 3.5%, about 3%, about 2.5%, about 2%, about 1.5%, about 1%, or about 0.5% sulfated polysaccharide provided as one component of the combination.

### C. Probiotic

In another aspect, a combination of the invention may optionally comprise one or more probiotics. For example, a combination of the invention may further comprise at least 1, at least 2, at least 3, at least 4, or at least 5 probiotics (each in an equal or varying amount).

A probiotic may be a symbiotic microbe. As used herein, the phrase “symbiotic microbe” refers to a bacterium whose presence in the gut provides a benefit or advantage to D. piger. The presence of D. piger may or may not provide a benefit to the symbiotic microbe. Typically, the symbiotic microbe provides a nutrient or some other substance that D. piger may use for growth or that promotes D. piger colonization in the gut. Alternatively, the symbiotic microbe may remove a nutrient or some other substance that negatively impacts D. piger growth or colonization in the gut. In some embodiments, a symbiotic microbe is a saccharolytic bacterial species. A saccharolytic bacterium is capable of hydrolyzing or otherwise breaking down a sugar molecule. Non-limiting examples of saccharolytic bacterial species include those belonging to the genera Bacteroides, Alistipes, Parabacteroides, Roseburia, Eubacterium, and Ruminococcus. Suitable isolated Bacteroides species may include, but are not limited to, B. acidifaciens, B. amylophilus, B. asaccharolyticus, B. barnesiae, B. bivius, B. buccae, B. buccalis, B. caccae, B. capillosus, B. capillus, B. cellulosilyticus, B. cellulosolvens, B. chinchilla, B. clarus, B. coagulans, B. coprocola, B. coprophilus, B. coprosuis, B. corporis, B. denticola, B. disiens, B. distasonis, B. dorei, B. eggerthii, B. endodontalis, B. faecichinchillae, B. faecis, B. finegoldii, B. fluxus, B. forsythus, B. fragilis, B. furcosus, B. galacturonicus, B. gallinarum, B. gingivalis, B. goldsteinii, B. gracilis, B. graminisolvens, B. helcogenes, B. heparinolyticus, B. hypermegas, B. intermedius, B. intestinalis, B. johnsonii, B. levii, B. loescheii, B. macacae, B. massiliensis, B. melaninogenicus, B. merdae, B. microfusus, B. multiacidus, B. nodosus, B. nordii, B. ochraceus, B. oleiciplenus, B. oralis, B. oris, B. oulorum, B. ovatus, B. paurosaccharolyticus, B. pectinophilus, B. pentosaceus, B. plebeius, B. pneumosintes, B. polypragmatus, B. praeacutus, B. propionicifaciens, B. putredinis, B. pyogenes, B. reticulotermitis, B. rodentium, B. ruminicola, B. salanitronis, B. salivosus, B. salyersiae, B. sartorii, B. splanchnicus, B. stercorirosoris, B. stercoris, B. succinogenes, B. suis, B. tectus, B. termitidis, B. thetaiotaomicron, B. uniformis, B. ureolyticus, B. veroralis, B. vulgatus, B. xylanisolvens, B. xylanolyticus, and B. zoogleoformans. Suitable isolated Afistipes species may include, but are not limited to A. finegoldii, A. indistinctus, A. onderdonkii, A. shahii, and A. putredinis. Suitable isolated Parabacteroides species may include, but are not limited to, P. chartae, P. distasonis, P. goldsteinii, P. gordonii, P. johnsonii, and P. merdae.

In other embodiments, a symbiotic microbe may be a bacterial species capable of liberating one or more sources of sulfate present in the gut of a subject, thereby providing an in vivo source of sulfate for D. piger. Sources of sulfate present in the gut of a subject may include, but are not limited to, a form of sulfate provided by the subject's diet, sulfated oligosaccharide side chains of glycosaminoglycans in a subject's mucins, and sulfonic acid moieties in bile acid. Accessing these sources of sulfate requires their liberation by sulfatases. Bacterial sulfatases require a sulfatase maturation enzyme for a post-translational modification (oxidation) of their active site cysteine or serine to a Cα-formylglycine. Non-limiting examples of bacterial species that can liberate sulfate includes those bacterial species with an active sulfatase, or those bacterial species comprising a nucleic acid sequence encoding a sulfatase and a nucleic acid sequence encoding a protein that can activate the sulfatase. The bacterial species may be native to the gut or not native to the gut. The symbiotic microbe may or may not be genetically engineered (i.e. a recombinant bacterium). In all cases the symbiotic microbe is isolated. In preferred embodiments, the bacterial species of the symbiotic microbe is Bacteroides thetaiotaomicron.

A probiotic may be present in a combination of the invention in from at least about 0.5% to 100% relative to the total weight (expressed as dry weight). For example, a probiotic of the invention may be present in a combination of the invention in about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0%, about 19.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 30.5%, about 31.0%, about 31.5%, about 32.0%, about 32.5%, about 33.0%, about 33.5%, about 34.0%, about 34.5%, about 35.0%, about 35.5%, about 36.0%, about 36.5%, about 37.0%, about 37.5%, about 38.0%, about 38.5%, about 39.0%, about 39.5%, about 40.0%, about 40.5%, about 41.0%, about 41.5%, about 42.0%, about 42.5%, about 43.0%, about 43.5%, about 44.0%, about 44.5%, about 45.0%, about 45.5%, about 46.0%, about 46.5%, about 47.0%, about 47.5%, about 48.0%, about 48.5%, about 49.0%, about 49.5%, about 50.0%, about 50.5%, about 51.0%, about 51.5%, about 52.0%, about 52.5%, about 53.0%, about 53.5%, about 54.0%, about 54.5%, about 55.0%, about 55.5%, about 56.0%, about 56.5%, about 57.0%, about 57.5%, about 58.0%, about 58.5%, about 59.0%, about 59.5%, about 60.0%, about 60.5%, about 61.0%, about 61.5%, about 62.0%, about 62.5%, about 63.0%, about 63.5%, about 64.0%, about 64.5%, about 65.0%, about 65.5%, about 66.0%, about 66.5%, about 67.0%, about 67.5%, about 68.0%, about 68.5%, about 69.0%, about 69.5%, about 70.0%, about 70.5%, about 71.0%, about 71.5%, about 72.0%, about 72.5%, about 73.0%, about 73.5%, about 74.0%, about 74.5%, about 75.0%, about 75.5%, about 76.0%, about 76.5%, about 77.0%, about 77.5%, about 78.0%, about 78.5%, about 79.0%, about 79.5%, about 80.0%, about 80.5%, about 81.0%, about 81.5%, about 82.0%, about 82.5%, about 83.0%, about 83.5%, about 84.0%, about 84.5%, about 85.0%, about 85.5%, about 86.0%, about 86.5%, about 87.0%, about 87.5%, about 88.0%, about 88.5%, about 89.0%, about 89.5%, about 90.0%, about 90.5%, about 91.0%, about 91.5%, about 92.0%, about 92.5%, about 93.0%, about 93.5%, about 94.0%, about 94.5%, about 95.0%, about 95.5%, about 96.0%, about 96.5%, about 97.0%, about 97.5%, about 98.0%, about 98.5%, about 99.0%, about 99.5%, or about 100% relative to the total weight (expressed as dry weight). Alternatively, a composition according to the invention may comprise from about 20¹ to about 20⁹ cfu/g of live microorganisms per gram of composition, or equivalent doses calculated for inactivated or dead microorganisms or for microorganism fractions or for produced metabolites.

### D. Additional Components

In other embodiments, the prebiotic is a polysaccharide that when hydrolyzed or otherwise broken down produces butyrate. Stated another way, the polysaccharide provides a source of fermentable carbohydrates that yields butyrate as an end product of fermentation. In an exemplary embodiment, the prebiotic is starch.

In another aspect, the present invention encompasses a composition that comprises at least one other component that may change the representation of sulfate-reducing bacteria in the gut. In some embodiments, the at least one other component is an antibiotic. Preferably, the antibiotic is preferentially cytotoxic or cytostatic to sulfate-reducing bacteria, bacteria of the genus Desulfovibrio, or bacteria of the class δ-Proteobacteria.

### E. Preferred Embodiments

In some preferred embodiments, a combination of the invention comprises at least one sulfated polysaccharide and at least one isolated SRB species selected from the group consisting of a D. piger and a bacterial species with at least one comparable in vivo fitness determinant to D. piger, wherein the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In exemplary embodiments, a sulfated polysaccharide is selected from the group consisting of a pentosan polysulfate, a fucoidan, a carrageenan, a sulfated glycosaminoglycan, and derivatives thereof.

In other preferred embodiments, a combination of the invention comprises at least one sulfated polysaccharide, at least one isolated bacterial species that liberates one or more sources of sulfate present in the gut of a subject, and at least one isolated SRB species selected from the group consisting of D. piger and a bacterial species with at least one comparable in vivo fitness determinant to D. piger, wherein the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In exemplary embodiments, a sulfated polysaccharide is selected from the group consisting of a pentosan polysulfate, a fucoidan, a carrageenan, a sulfated glycosaminoglycan, and derivatives thereof.

In other preferred embodiments, a combination of the invention comprises at least one sulfated polysaccharide and at least one isolated Desulfovibrio species comprising a nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In exemplary embodiments, a sulfated polysaccharide is selected from the group consisting of a pentosan polysulfate, a fucoidan, a carrageenan, a sulfated glycosaminoglycan, and derivatives thereof.

In other preferred embodiments, a combination of the invention comprises at least one sulfated polysaccharide, at least one isolated bacterial species that liberates one or more sources of sulfate present in the gut of a subject, and at least one isolated Desulfovibrio species comprising a nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In exemplary embodiments, a sulfated polysaccharide is selected from the group consisting of a pentosan polysulfate, a fucoidan, a carrageenan, a sulfated glycosaminoglycan, and derivatives thereof.

### F. Formulations

In each of the above embodiments, at least one SRB species, at least one sulfated polysaccharide and, when present, symbiotic microbes and nutrients (each a “component”) may be formulated for animal or human use. In some embodiments, each component is formulated separately. In other embodiments, two or more components are formulated together. In still other embodiments, all components are formulated together. The one or more formulations may then be processed into one or more dosage forms that can be administered together, sequentially, or over a period of time (for example, over 1 minute, 10 minutes, 30 minutes, 1 hour, 3 hours, 6 hours, 9 hours, 12 hours, 18 hours, 24 hours, or more). Administration can be performed using standard effective techniques, including oral, parenteral (e.g. intravenous, intraperitoneal, subcutaneous, intramuscular), buccal, sublingual, or suppository administration. The term orally, as used herein, refers to any form of administration by mouth, including addition of a composition to animal feed or other food product. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Methods for preparing compositions comprising probiotics are well known in the art, and commercially available probiotics are available in liquid and dry formulations. Generally speaking, any method known in the art is suitable, provided the viability of the microorganism is significantly preserved. Several approaches have been investigated for improving the technological and therapeutic performance of probiotics, including strain selection and probiotic stabilization during spray drying and/or freeze drying and gastric transit, as described in Ross et al. Journal of Applied Microbiology (2005) 98:1410-1417, Kosin et al. Food Technology and Biotechnology (2006) 44(3): 371-379, Riaz et al. Crit Rev Food Sci Nutr (2013) 53(3): 231-44; and Ledeboer et al “Technological aspects of making live, probiotic-containing gut health foods” www.labip.com/uploads/media/GutImpact_I_finalversion_EDM.pdf; each hereby incorporated by reference in its entirety.

Methods of preparing compositions for animal or human use are also well known in the art. For instance, a composition may be generally formulated as a liquid composition, a solid composition or a semi-solid composition. Liquid compositions include, but are not limited to, aqueous suspensions, solutions, emulsions, elixirs, or syrups. Liquid composition will typically include a solvent carrier selected from a polar solvent, a non-polar solvent, or a combination of both. The choice of solvent will be influenced by the properties of the components of the composition. For example, if the components are water-soluble, a polar solvent may be used. Alternatively, if the components of the composition are lipid-soluble, a non-polar solvent may be used. Suitable polar and non-polar solvents are known in the art. Semi-solid compositions include douches, suppositories, creams, and topicals. Dry compositions include, but are not limited to, reconstitutable powders, chewable tablets, quick dissolve tablets, effervescent tablets, multi-layer tablets, bi-layer tablets, capsules, soft gelatin capsules, hard gelatin capsules, caplets, lozenges, chewable lozenges, beads, powders, granules, particles, microparticles, and dispersible granules. Formulations may include a combination of the invention along with an excipient. Non-limiting examples of excipients include binders, diluents (fillers), disintegrants, effervescent disintegration agents, preservatives (antioxidants), flavor-modifying agents, lubricants and glidants, dispersants, coloring agents, pH modifiers, chelating agents, antimicrobial agents, release-controlling polymers, and combinations of any of these agents.

Non-limiting examples of binders suitable for the formulations of various embodiments include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohols, polyethylene glycol, polyols, saccharides, oligosaccharides, polypeptides, oligopeptides, and combinations thereof. The polypeptide may be any arrangement of amino acids ranging from about 200 to about 300,000 Daltons. In one embodiment, the binder may be introduced into the mixture to be granulated in a solid form including but not limited to a crystal, a particle, a powder, or any other finely divided solid form known in the art. In another embodiment, the binder may be dissolved or suspended in a solvent and sprayed onto the mixture in a granulation device as a binder fluid during granulation.

Non-limiting examples of diluents (also referred to as “fillers” or “thinners”) include carbohydrates, inorganic compounds, and biocompatible polymers, such as polyvinylpirrolydone (PVP). Other non-limiting examples of diluents include dibasic calcium sulfate, tribasic calcium sulfate, starch, calcium carbonate, magnesium carbonate, microcrystalline cellulose, dibasic calcium phosphate, tribasic calcium phosphate, magnesium carbonate, magnesium oxide, calcium silicate, talc, modified starches, saccharides such as sucrose, dextrose, lactose, microcrystalline cellulose, fructose, xylitol, and sorbitol, polyhydric alcohols; starches; pre-manufactured direct compression diluents; and mixtures of any of the foregoing.

Disintegrents may be effervescent or non-effervescent. Non-limiting examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Suitable effervescent disintegrants include but are not limited to sodium bicarbonate in combination with citric acid, and sodium bicarbonate in combination with tartaric acid.

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof. In an exemplary embodiment, the preservatives is an antioxidant, such as a-tocopherol or ascorbate, and antimicrobials, such as parabens, chlorobutanol or phenol.

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include but are not limited to cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IRO; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, additional taste-masking agents contemplated are those described in U.S. Pat. Nos. 4,851,226, 5,075,114, and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

Lubricants may be utilized to lubricate ingredients that form a composition of the invention. As a glidant, the lubricant facilitates removal of solid dosage forms during the manufacturing process. Non-limiting examples of lubricants and glidants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. The composition will generally comprise from about 0.01% to about 20% by weight of a lubricant. In some embodiments, the composition will comprise from about 0.1% to about 5% by weight of a lubricant. In a further embodiment, the composition will comprise from about 0.5% to about 2% by weight of a lubricant.

Dispersants may include but are not limited to starch, alginic acid, polyvinylpyrrolidones, guar gum, kaolin, bentonite, purified wood cellulose, sodium starch glycolate, isoamorphous silicate, and microcrystalline cellulose as high hydrophilic-lipophilic balance (HLB) emulsifier surfactants.

Depending upon the embodiment, it may be desirable to include a coloring agent. Suitable color additives include but are not limited to food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in various embodiments.

Non-limiting examples of pH modifiers include citric acid, acetic acid, tartaric acid, malic acid, fumaric acid, lactic acid, phosphoric acid, sorbic acid, benzoic acid, sodium carbonate and sodium bicarbonate.

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

An antimicrobial agent may be included as an excipient to minimize the degradation of the compound according to this disclosure by microbial agents, including but not limited to bacteria and fungi. Non-limiting examples of antimicrobials include parabens, chlorobutanol, phenol, calcium propionate, sodium nitrate, sodium nitrite, Na2EDTA, and sulfites including but not limited to sulfur dioxide, sodium bisulfite, and potassium hydrogen sulfite.

Release-controlling polymers may be included in the various embodiments of the solid dosage compositions incorporating compounds according to this disclosure. In one embodiment, the release-controlling polymers may be used as a tablet coating. In other embodiments, including but not limited to bilayer tablets, a release-controlling polymer may be mixed with the granules and other excipients prior to the formation of a tablet by a known process including but not limited to compression in a tablet mold. Suitable release-controlling polymers include but are not limited to hydrophilic polymers and hydrophobic polymers.

Suitable hydrophilic release-controlling polymers include, but are not limited to, cellulose acetate, cellulose diacetate, cellulose triacetate, cellulose ethers, hydroxyethyl cellulose, hydroxypropyl cellulose, hydroxypropyl methylcellulose, microcrystalline cellulose, nitrocellulose, crosslinked starch, agar, casein, chitin, collagen, gelatin, maltose, mannitol, maltodextrin, pectin, pullulan, sorbitol, xylitol, polysaccharides, ammonia alginate, sodium alginate, calcium alginate, potassium alginate, propylene glycol alginate, alginate sodium carmellose, calcium carmellose, carrageenan, fucoidan, furcellaran, arabicgum, carrageensgum, ghaftigum, guargum, karayagum, locust beangum, okragum, tragacanthgum, scleroglucangum, xanthangum, hypnea, laminaran, acrylic polymers, acrylate polymers, carboxyvinyl polymers, copolymers of maleic anhydride and styrene, copolymers of maleic anhydride and ethylene, copolymers of maleic anhydride propylene or copolymers of maleic anhydride isobutylene), crosslinked polyvinyl alcohol and poly N-vinyl-2-pyrrolidone, diesters of polyglucan, polyacrylamides, polyacrylic acid, polyamides, polyethylene glycols, polyethylene oxides, poly(hydroxyalkyl methacrylate), polyvinyl acetate, polyvinyl alcohol, polyvinyl chloride, polystyrenes, polyvinylpyrrolidone, anionic and cationic hydrogels, and combinations thereof.

The invention can also include compositions that can be created as a powder that can be added to food items, as a baked good (e.g., as cookies and brownies), and as a concentrate. The concentrate can be added to water or another ingestible liquid to create a nutritional beverage. The nutritional supplement is typically contained within a one-serving or multiple serving container such as a package, box, carton, wrapper, bottle or can. Where the nutritional supplement is prepared in the form of a concentrate that can be added to and mixed with a beverage, a bottle or can be used for packaging the concentrate. The nutritional supplement can also include water.

II. Method for Increasing the Representation of D. Piger or an SRB Species with at Least One Comparable In Vivo Fitness Determinant to D. Piger in the Gut of a Subject

When administered to a subject, combinations of the invention described above in Section I may increase in the gut of the subject the representation of D. piger or an SRB species with at least one comparable in vivo fitness determinant to D. piger. Applicants show in the Examples that although free sulfate in the diet is not a required determinant of D. piger levels in the intestine, supplementation of the diet with a sulfated polysaccharide significantly increases D. piger levels in the fecal microbiota relative to an unsupplemented diet. Thus, in another aspect, the present invention provides a method for increasing the representation of D. piger or an SRB species with at least one comparable in vivo fitness determinant to D. piger in the gut of a subject. Typically the method comprises administering a combination of the invention in an effective amount to a subject and, optionally, confirming an increase representation of D. piger or an SRB species with at least one comparable in vivo fitness determinant to D. piger. Suitable subjects are described above. In certain embodiments, a subject is as described in Section III(A).

In some embodiments, a combination of the invention comprises at least one sulfated polysaccharide and at least one isolated SRB species selected from the group consisting of D. piger and a bacterial species with at least one comparable in vivo fitness determinant to D. piger, wherein the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In other embodiments, a combination of the invention comprises at least one sulfated polysaccharide and at least one isolated Desulfovibrio species comprising a nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In certain embodiments, combinations of the invention further comprise at least one symbiotic microbe. In preferred embodiments, a sulfated polysaccharide is selected from the group consisting of a dextran sulfate, a pentosan polysulfate, a fucoidan, a carrageenan, a sulfated glycosaminoglycan, and derivatives thereof. In an exemplary embodiment, a sulfated polysaccharide is chondroitin sulfate.

Confirming an increased representation of D. piger or an SRB species with at least one comparable in vivo fitness determinant to D. piger following administration of a combination of the invention requires measuring the abundance of the species in a sample comprising the subject's microbiota before and after administration of the combination, and comparing the levels of abundance to determine the presence and direction of change. If the abundance is greater after administration relative to before administration, then representation increased. Generally speaking, such methods employ qualitative, semi-quantitative or quantitative techniques, of which many are known in the art. See for example, Muyzer G and Stams A J Nature Review Microbiology 2010; 6:441-454. When bacteria are culturable, a sample may be collected, processed, plated on appropriate growth media, cultured under suitable conditions (i.e. temperature, presence or absence of oxygen and carbon dioxide, presence or absence of agitation, etc.), and colony forming units may be determined. Culture-independent methods that provide a comparative analysis of the presence or abundance of nucleic acid sequence at the genus-level or species-level, however, are preferred. Such methods include, but are not limited to, high throughput amplicon sequencing, quantitative-PCR, array-based methods, and fluorescence in situ hybridization (FISH). Many different probes or primers can be designed to target nucleic acid sequences of different taxonomic groups of SRB species. For example, a suitable threshold for genus classification is that genus-level phylotypes share ≧70% identity over a given region, preferably ≧80%, more preferably ≧95%. A suitable threshold for species classification is that species-level phylotypes share ≧90% identity over a given region, preferably ≧94%, more preferably ≧97%. Nucleic acids that may be queried include, but are not limited to, 16s rRNA, nucleic acid sequences encoding a polypeptide involved in the sulfate-reduction pathway, nucleic acid sequences encoding a polypeptide involved in hydrogen consumption, or combinations thereof. In certain embodiments, the proportional representation of one or more SRB species is calculated by determining the abundance of one or more nucleic acid sequences encoding an enzyme selected from the group consisting of DsrA, DsrB, DsrD, DsrJ, DsrK, DsrM, DsrO, DsrP, AprA, AprB, Sat, QmoA, QmoB, QmoC, HysA, HysB or a combination thereof. Example 1 illustrates, using aprA, how primers can be designed to amplify a nucleic acid sequence present in all known SRB species and amplicons can be generated from fecal samples. The same method may be used for other nucleic acid sequences.

Preferable samples comprising a subject's microbiota may include, but are not limited to, a fecal sample or a sample of the luminal contents of the gut. Methods of obtaining and processing a fecal sample and a sample of the lumenal contents are known in the art and further detailed in the Examples.

Typically, an effective amount of a combination increases the representation of the SRB species by at least 10%. For example, the amount of an indicator may be increased by at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99, or 100%. In some embodiments, the representation of the SRB species may increase about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%. In other embodiments, the representation of the SRB species may increase at least 2-fold, at least 5-fold, at least 10-fold, at least 20-fold, at least 50-fold, or at least 100-fold. The representation of the SRB species may be measured about 1 day to about 14 days after administration of the combination of the invention, including at 1 day, 2 days, 3 days, 4 days, 5 days, 6 days, 7 days, 8 days, 9 days, 10 days, 11 days, 12 days 13 days or 14 days after administration of the combination. For example, the representation of the SRB species may be measured about 1-5 days, about 1-7 days, 5-14 days, about 7-14 days, about 10-14 days, about 1-3 days, about 3-6 days, about 4-7 days, about 5-8 days, about 6-9 days, about 7-10 days, about 8-11 days, about 9-12 days, about 10-13 days, about 11-14 days, or about 12-14 days after administration.

### III. Method for Increasing Microbial Fermentative Activity in the Gut of a Subject in Need Thereof

As noted above and further detailed in the Examples, Applicants have discovered that changes in the representation of D. piger in the gut of a subject affects microbial fermentative activity. Thus, in another aspect, the present invention provides a method for increasing microbial fermentative activity in the gut of a subject in need thereof. Typically the method comprises identifying a subject in need, administering a combination of the invention in an effective amount to the identified subject, and, optionally, confirming an increase in microbial fermentative activity following administration of the combination.

Increased microbial fermentative activity improves the biotransformation of foods, such that more energy (i.e. more calories) is extracted and less energy passes through the system. Therefore, in another aspect, the present invention provides a method for increasing the nutritional value of a diet. The method comprises administering to a subject as part of a diet a combination of the invention, wherein the combination increases microbial fermentative activity in the gut of the subject, thereby increasing the nutritional value of the diet. Numerous methods exist the art to determine the energy value of food and the energy value extracted by a subject. For example, one may compare the energy available in a food to the energy present in a subject's excrement (urine and/or feces) after ingestion of the food. For further details, see “Energy Value of Foods . . . basis and derivation” by Annabel L. Meriil and Bernice K Watt, incorporated herein by reference (http://www.ars.usda.gov/SP2UserFiles/Place/12354500/Data/Classics/ah74.pdf). Increasing the nutritional value of a diet by improving the biotransformation of foods consumed by a subject may also increase a subject's body mass.

### A. Subject in Need

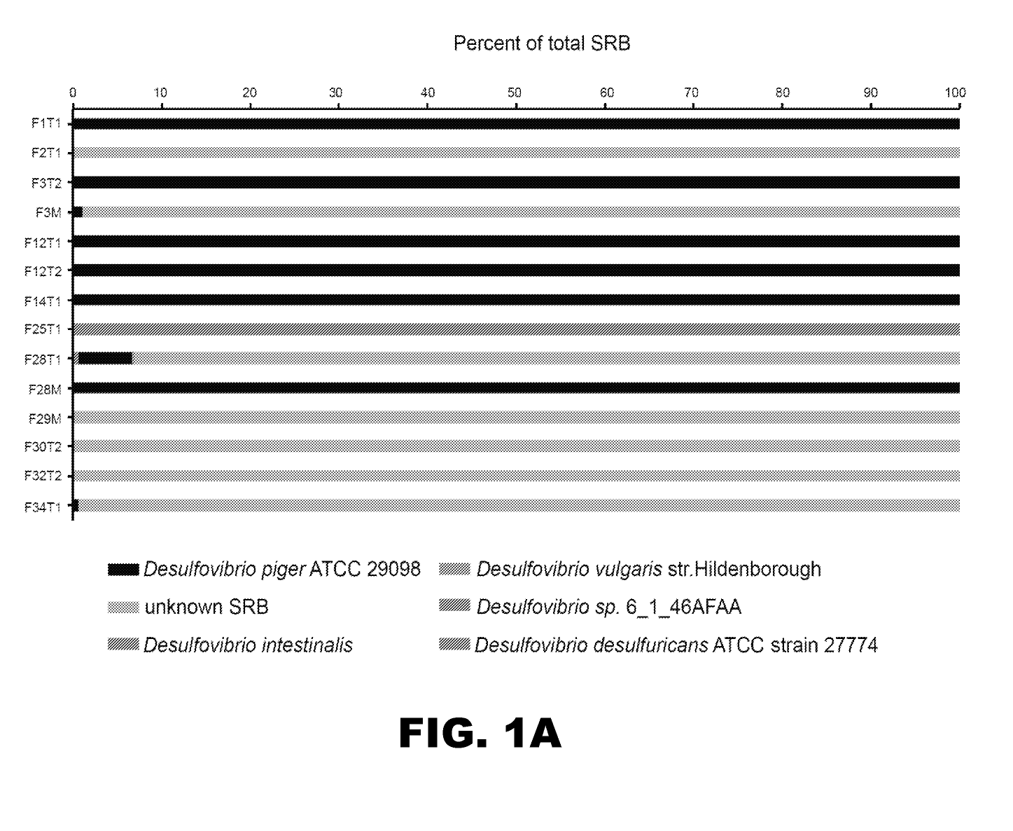

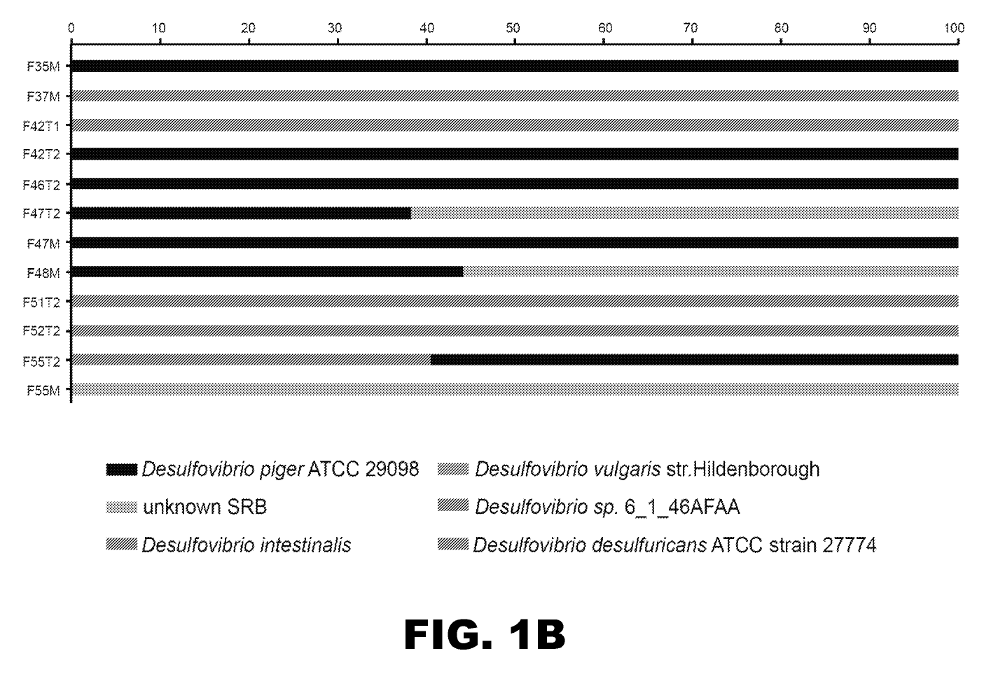

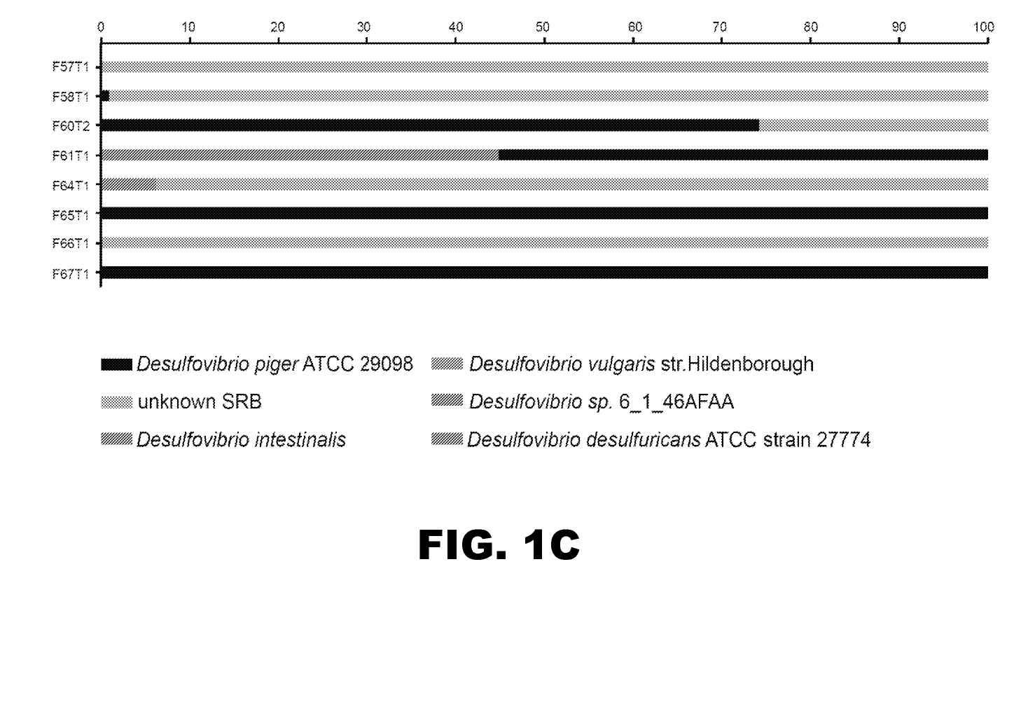

There is considerable variation in SRB species carriage between subjects, even when looking within a single genus (see FIG. 1). Generally speaking, a subject in need of increased microbial fermentative activity may have a decreased proportional representation of SRB species in the gut. Proportional representation may be calculated by comparing the abundance of an SRB genus or species relative to (i) the abundance of total gut mircobiota, (ii) the abundance of total sulfur reducing bacteria, or (iii) the abundance of an SRB genus. Proportional representation may also be calculated by comparing the abundance of all sulfate-reducing bacteria relative to the abundance of total gut mircobiota. Methods for measuring the abundance of sulfate-reducing bacteria are described above in Section II. Alternatively, a subject in need of increased microbial fermentative activity may have a decreased proportional representation of hydrogen consuming bacteria in the gut. Methods for measuring the abundance of hydrogen-consuming bacteria are similar to those described for measuring the abundance of sulfate-reducing bacteria in Section II. The choice of nucleic acid sequence may or may not be the same for detecting sulfate-reducing bacteria compared to hydrogen-consuming bacteria. Not all sulfate-reducing bacteria may be capable of consuming hydrogen and not all hydrogen-consuming bacteria may be capable of sulfate-reduction. For example, a nucleic acid sequence encoding AprA is suitable choice for detecting SRB species but is not suitable for detecting all hydrogen-consuming bacteria, as this will group of bacteria will also include acetogens and methanogens. A skilled artisan will appreciate that there may be no single nucleic acid sequence to calculate the abundance of acetogens, methanogens and sulfate-reducing bacteria, though a limited combination is possible. Other methods known in the art for determine the relative abundance of hydrogen consuming bacteria may also be used, including hydrogen breath tests.

In some embodiments, the proportional representation of hydrogen-consuming bacteria in a gut microbiota sample obtained from a subject in need of increased microbial fermentative activity may be less than about 20% of the total gut microbiota, including about 0%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, or about 19%, of the total gut microbiota. In other embodiments, the proportional representation of hydrogen-consuming bacteria in a gut microbiota sample obtained from a subject in need of increased microbial fermentative activity may be about 2-fold, 3-fold, 4-fold, 5-fold, 6-fold, 7-fold, 8-fold, 9-fold, 10-fold or more less than average abundance of hydrogen consuming bacteria in a subject. For example, sulfate-reducing bacteria and methanogens typically account for about 2% of the total gut microbiota and hydrogen-consuming acetogens account for about 10-20% of the total gut microbiota.

In some embodiments, the proportional representation of sulfate-reducing bacteria in a gut microbiota sample obtained from a subject in need of increased microbial fermentative activity may be less than about 1% of the total gut microbiota, including about 0%, about 0.1%, about 0.2%, about 0.3%, about 0.4%, about 0.5%, about 0.6%, about 0.7%, about 0.8%, about 0.9% of the total gut microbiota. In other embodiments, the proportional representation of sulfate-reducing bacteria in a gut microbiota sample obtained from a subject in need of increased microbial fermentative activity may be about 2-fold, 3-fold, 4-fold, 5-fold, 6-fold, 7-fold, 8-fold, 9-fold, 10-fold or more less than average abundance of sulfate-reducing bacteria in a subject. For example, sulfate-reducing bacteria typically account for about 1-2% of the total gut microbiota.

In some embodiments, the proportional representation of Desulfovibrio bacteria in a gut microbiota sample obtained from a subject in need of increased microbial fermentative activity may be less than 100% of total sulfate-reducing bacteria, including about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% of total sulfate-reducing bacteria. In other embodiments, the proportional representation of Desulfovibrio bacteria in a gut microbiota sample obtained from a subject in need of increased microbial fermentative activity may be about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100% of total sulfate-reducing bacteria. In still other embodiments, the proportional representation of Desulfovibrio bacteria in a gut microbiota sample obtained from a subject in need of increased microbial fermentative activity may be less than about 10%, less than about 20%, less than about 30%, less than about 40%, less than about 50%, less than about 60%, less than about 70%, less than about 80%, less than about 90%, or less than about 95% of total sulfate-reducing bacteria.

In some embodiments, the proportional representation of D. piger in a gut microbiota sample obtained from a subject in need of increased microbial fermentative activity may be less than 100% of total sulfate-reducing bacteria, including about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% of total sulfate-reducing bacteria. In other embodiments, the proportional representation of D. piger in a gut microbiota sample obtained from a subject in need of increased microbial fermentative activity may be about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100% of total sulfate-reducing bacteria. In still other embodiments, the proportional representation of D. piger in a gut microbiota sample obtained from a subject in need of increased microbial fermentative activity may be less than about 10%, less than about 20%, less than about 30%, less than about 40%, less than about 50%, less than about 60%, less than about 70%, less than about 80%, less than about 90%, or less than about 95% of total sulfate-reducing bacteria. In some embodiments, the proportional representation of D. piger in a gut microbiota sample obtained from a subject in need of increased microbial fermentative activity may be less than about 75% of total sulfate-reducing bacteria.

In some embodiments, the proportional representation of D. piger in a gut microbiota sample obtained from a subject in need of increased microbial fermentative activity may be less than 100% of total Desulfovibrio bacteria, including about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% of total sulfate-reducing bacteria. In other embodiments, the proportional representation of D. piger in a gut microbiota sample obtained from a subject in need of increased microbial fermentative activity may be about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100% of total Desulfovibrio bacteria. In still other embodiments, the proportional representation of D. piger in a gut microbiota sample obtained from a subject in need of increased microbial fermentative activity may be less than about 10%, less than about 20%, less than about 30%, less than about 40%, less than about 50%, less than about 60%, less than about 70%, less than about 80%, less than about 90%, or less than about 95% of total Desulfovibrio bacteria.

In some embodiments, the proportional representation of bacteria belonging to an SRB species with at least one comparable in vivo fitness determinant to D. piger in a gut microbiota sample obtained from a subject in need of increased microbial fermentative activity may be less than 100% of total sulfate-reducing bacteria, including about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% of total sulfate-reducing bacteria. In other embodiments, the proportional representation of bacteria belonging to an SRB species with at least one comparable in vivo fitness determinant to D. piger in a gut microbiota sample obtained from a subject in need of increased microbial fermentative activity may be about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100% of total sulfate-reducing bacteria. In still other embodiments, the proportional representation of bacteria belonging to an SRB species with at least one comparable in vivo fitness determinant to D. piger in a gut microbiota sample obtained from a subject in need of increased microbial fermentative activity may be less than about 10%, less than about 20%, less than about 30%, less than about 40%, less than about 50%, less than about 60%, less than about 70%, less than about 80%, less than about 90%, or less than about 95% of total sulfate-reducing bacteria. In each of the above embodiments, the at least one comparable in vivo fitness determinant may be selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). Alternatively, in each of the above embodiments, the at least one comparable in vivo fitness determinant may be as defined in Section I.

In some embodiments, the proportional representation of bacteria belonging to an SRB species with at least one comparable in vivo fitness determinant to D. piger in a gut microbiota sample obtained from a subject in need of increased microbial fermentative activity may be less than 100% of total Desulfovibrio bacteria, including about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% of total sulfate-reducing bacteria. In other embodiments, the proportional representation of bacteria belonging to an SRB species with at least one comparable in vivo fitness determinant to D. piger in a gut microbiota sample obtained from a subject in need of increased microbial fermentative activity may be about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100% of total Desulfovibrio bacteria. In still other embodiments, the proportional representation of bacteria belonging to an SRB species with at least one comparable in vivo fitness determinant to D. piger in a gut microbiota sample obtained from a subject in need of increased microbial fermentative activity may be less than about 10%, less than about 20%, less than about 30%, less than about 40%, less than about 50%, less than about 60%, less than about 70%, less than about 80%, less than about 90%, or less than about 95% of total Desulfovibrio bacteria. Preferably, in each of the above embodiments, the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). Alternatively, in each of the above embodiments, the at least one comparable in vivo fitness determinant may be as defined in Section I.

### B. Administering a Combination of the Invention

As noted above in Section 1(F), combinations of the invention may be formulated for animal or human use. One or more formulations comprising the components of the combination may then be processed into one or more dosage forms that can be administered together, sequentially, or over a period of time (for example, over 1 minute, 10 minutes, 30 minutes, 1 hour, 3 hours, 6 hours, 9 hours, 12 hours, 18 hours, 24 hours, or more). Administration can be performed using standard effective techniques, including oral, parenteral (e.g. intravenous, intraperitoneal, subcutaneous, intramuscular), buccal, sublingual, or suppository administration.

In some embodiments, a combination of the invention comprises at least one sulfated polysaccharide and at least one isolated SRB species selected from the group consisting of a D. piger and a bacterial species with at least one comparable in vivo fitness determinant to D. piger, wherein the at least one comparable in vivo fitness determinant is selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In other embodiments, a combination of the invention comprises at least one sulfated polysaccharide and at least one isolated Desulfovibrio species comprising a nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12). In certain embodiments, combinations of the invention further comprise at least one symbiotic microbe. In preferred embodiments, a sulfated polysaccharide is selected from the group consisting of a dextran sulfate, a pentosan polysulfate, a fucoidan, a carrageenan, a sulfated glycosaminoglycan, and derivatives thereof. In an exemplary embodiment, a sulfated polysaccharide is chondroitin sulfate.

### C. Confirming an Increase in Microbial Fermentative Activity

Proteins and carbohydrates are broken down by primary fermenters, yielding short-chain fatty acids (e.g., acetate, propionate, and butyrate) and gases (e.g., H₂ and CO₂). In one aspect, an increase in microbial fermentative activity may be confirmed by measuring the amount of short-chain fatty acids in a sample obtained from a subject before and after administration of a combination of the invention, and comparing the amount to determine the presence and direction of change. A greater amount of short chain fatty acids in a sample after administration relative to before administration indicates an increase in microbial fermentative activity.

One challenge primary fermentators and other microbes face during fermentation is to maintain redox balance while maximizing their energy production. Many species have branched fermentation pathways that allow for disposal of reducing equivalents; producing H₂ is an energetically efficient way of doing so, yielding higher levels of ATP. SRB species are capable of using H₂ as an electron donor and sulfate as the terminal electron acceptor for growth, in the process producing hydrogen sulfide. Therefore, in another aspect, an increase in microbial fermentative activity may be confirmed by measuring the amount of hydrogen sulfide and/or the abundance of the administered SRB species in a sample obtained from a subject before and after administration of a combination of the invention, and comparing the amount to determine the presence and direction of change. A greater amount of one or both in a sample after administration relative to before administration indicates an increase microbial fermentative activity. In another aspect, an increase in microbial fermentative activity can be confirmed by measuring the redox potential of a sample obtained from a subject before and after administration of a combination of the invention, and comparing the levels to determine the presence and direction of change. A lower redox potential in a sample after administration relative to before administration indicates an increase microbial fermentative activity.

Typically, an effective amount of a combination increases microbial fermentative activity, as measured by an increase an indicator selected from the group consisting of H2S, short chain fatty acids, abundance of SRB, by at least 10%. For example, the amount of an indicator may be increased by at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99, or 100%. In some embodiments, the amount of an indicator is increased about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%. In other embodiments, an amount of an indicator is increased at least 2-fold, at least 5-fold, at least 10-fold, at least 20-fold, at least 50-fold, or at least 100-fold. The amount of the indicator can be measured about 1 day to about 14 days after administration of the combination of the invention. For example, the amount of the indicator can be measured about 1-5 days, about 1-7 days, 5-14 days, about 7-14 days, about 10-14 days, about 1-3 days, about 3-6 days, about 4-7 days, about 5-8 days, about 6-9 days, about 7-10 days, about 8-11 days, about 9-12 days, about 10-13 days, about 11-14 days, or about 12-14 days after administration. Methods of measuring the abundance of sulfate-reducing bacteria are described in Section II. Methods of measuring hydrogen sulfide and short chain fatty acids are known in the art and further detailed in the Examples. Suitable methods may include, but are not limited to, gas chromatography-mass spectrometry, liquid chromatography-mass spectrometry, and high performance liquid chromatography.

### D. Other Aspects

Combinations of the invention may be used with or without changes to a subject's diet. In some embodiments, a combination of the invention is used without a change to a subject's diet. In other embodiments, a combination of the invention is used with a change to a subject's diet. Suitable changes will be apparent to a skilled artisan and will vary depending on the subject and the type of beneficial effect desired. Non-limiting examples of changes to a diet may include, but are not limited to, a change in the type or amount of a food, an increase in daily caloric content, a decrease in daily caloric content, an increase in daily saturated and/or unsaturated fat intake, a decrease in daily saturated and/or unsaturated fat intake, an increase in the amount of starchy foods consumed daily, a decrease in the amount of starchy foods consumed daily, an increase in the amount of foods high in sulfate (e.g. commercial breads, dried fruits and vegetables, nuts fermented beverages, and brassica vegetables), a decrease in the amount of foods high in sulfate, an increase in the amount of plant-based (or plant-derived) polysaccharides consumed daily, and a decrease in the amount of plant-based (or plant-derived) polysaccharides consumed daily.

### IV. Method for Classifying a Compound Administered to a Subject as Effective or Ineffective

In another aspect, the present invention encompasses a method for classifying a compound administered to a subject as effective or ineffective, wherein the desired effect is an increase in microbial fermentative activity and/or an increase in the biotransformation of food or nutrients in the gut of a subject. Typically, the method comprises (i) obtaining a sample from the subject before and after administration of the compound, (ii) determining the amount of at least one biomarker of microbial fermentative activity in each sample and calculating the change in the amount of the biomarker, and (iii) classifying the compound as effective if the change in the biomarker indicates microbial fermentative activity increased and classifying the compound as ineffective if the change in the biomarker indicates the microbial fermentative activity decreased or did not change at all.

In another aspect, the present invention encompasses a method for classifying a compound administered to a subject as effective or ineffective, wherein the desired effect is a decrease in microbial fermentative activity in the gut. Typically, the method comprises (i) obtaining a sample from the subject before and after administration of the compound, (ii) determining the amount of at least one biomarker of microbial fermentative activity in each sample and calculating the change in the amount of the biomarker, and (iii) classifying the compound as effective if the change in the biomarker indicates microbial fermentative activity decreased and classifying the compound as ineffective if the change in the biomarker indicates microbial fermentative activity increased or did not change at all.

In some embodiments, the amount of at least one biomarker of microbial fermentative activity is determined. For example, the amount of at least 1, at least 2, at least 3, at least 4, at least 5, at least 6, at least 7, at least 8, at least 9 or at least ten biomarkers is determined. Alternatively, the amount of at least 5, at least 10, at least 15, at least 20, at least 25, at least 30, at least 35, at least 40, at least 45, or at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 biomarkers may be determined.

Compounds administered to a subject may be a pharmaceutical, nutraceutical, probiotic, prebiotic, or dietary supplement, as well as compositions of the invention.

Preferable samples may include, but are not limited to, a fecal sample or luminal contents of the gut collected from a subject. Methods of obtaining and processing fecal samples and lumenal contents are known in the art and further detailed in the Examples. Suitable subjects are described above.

A change in the presence, absence or abundance of a biomarker of microbial fermentative activity is an appropriate measure of whether a composition or method of treatment is having the desired effect on microbial fermentation (i.e. increasing or decreasing microbial fermentative activity). Suitable biomarkers of the microbial fermentative activity may include, but are not limited to, hydrogen sulfide, short chain fatty acids, the abundance of hydrogen consuming bacteria, and a biomolecule present in, produced by, or modified by hydrogen consuming bacteria. Further details for measuring these biomarkers may be found above in Section II and Section III.

Non-limiting examples of short chain fatty acids include butyric acid, acetic acid and propionic acid. Methods of measuring hydrogen sulfide and short chain fatty acids are known in the art and further detailed in the Examples. Suitable methods may include, but are not limited to, gas chromatography-mass spectrometry, liquid chromatography-mass spectrometry, and high performance liquid chromatography. A skilled artisan will appreciate that other methods may be also be used. In some embodiments, the biomarker is hydrogen sulfide and an increase in hydrogen sulfide in a sample indicates an increase in microbial fermentative activity in the gut. In other embodiments, the biomarker is hydrogen sulfide and a decrease in hydrogen sulfide in a sample indicates a decrease in microbial fermentative activity in the gut. In still other embodiments, the biomarker is short chain fatty acids and an increase in short chain fatty acids in a sample indicates an increase in microbial fermentative activity in the gut. In yet other embodiments, the biomarker is short chain fatty acids and a decrease in short chain fatty acids in a sample indicates a decrease in microbial fermentative activity in the gut.

Hydrogen consuming bacteria in the gut may include methanogens, acetogens, and sulfate-reducing bacteria. In some embodiments, a hydrogen consuming bacterium is a methanogen. Methanogens are a clade of organisms unique to the domain Archaea and are named for their ability to oxidize hydrogen and reduce CO₂ to produce CH₄. Non-limiting examples of methanogens includes members of the genus Methanobrevibacter, Methanospaera, or Methanosarcina. In other embodiments, a hydrogen consuming bacterium is an acetogen. Acetogens are obligate anaerobes that synthesize the high energy intermediate acetyl-CoA from CO₂. Non-limiting examples of acetogens include Ruminococcus productus, Blautia hydrogenotrophica, and Marvinbryantia formatexigens. In still other embodiments, a hydrogen consuming bacterium is a sulfate-reducing bacterium. Suitable sulfate-reducing bacteria are described above. In an exemplary embodiment, the biomarker is a sulfate-reducing bacterium selected from the group consisting of D. piger and a bacterium with comparable in vivo fitness determinants to D. piger, and an increase in the biomarker in a sample indicates an increase in microbial fermentative activity in the gut. In another exemplary embodiment, the biomarker is a sulfate-reducing bacterium selected from the group consisting of D. piger and a bacterium with comparable in vivo fitness determinants to D. piger, and a decrease in the biomarker in a sample indicates a decrease in microbial fermentative activity in the gut.

Methods of measuring the presence, absence or change in abundance of hydrogen consuming bacteria are known in the art. For example, in embodiments where the bacteria are culturable, the sample may be collected, processed, plated on appropriate growth media, cultured under suitable conditions (i.e. temperature, presence or absence of oxygen and carbon dioxide, agitation, etc.), and colony forming units may be determined. Alternatively, in embodiments where the bacteria are not culturable or where it may be more convenient to use an approach with greater throughput, sequencing methods or arrays may be used. Such methods are well known in the art.

As used herein, “biomolecule” may refer to a nucleic acid, an oligonucleic acid, an amino acid, a peptide, a polypeptide, a protein, a lipid, a metabolite, or a fragment thereof. Nucleic acids may include RNA, DNA, and naturally occurring or synthetically created derivatives. A biomolecule may be present in, produced by, or modified by hydrogen consuming bacteria within the gut. In some embodiments, the biomolecule may be present in, produced by, or modified by acetogens. In other embodiments, the biomolecule may be present in, produced by, or modified by methanogens. In still other embodiments, the biomolecule may be present in, produced by, or modified by sulfate-reducing bacteria. In yet other embodiments, the biomolecule may be present in, produced by, or modified by sulfate-reducing bacteria selected from the group consisting of D. piger and a bacterium with comparable in vivo fitness determinants to D. piger. In an exemplary embodiment, the biomarker is a D. piger in vivo fitness determinant or a comparable D. piger in vivo fitness determinant, and an increase in the biomarker indicates an increase in microbial fermentative activity. In another exemplary embodiment, the biomarker is a D. piger in vivo fitness determinant or a comparable D. piger in vivo fitness determinant, and a decrease in the biomarker indicates a decrease in microbial f. Suitable D. piger in vivo fitness determinants are described above.

Methods for measuring the presence, absence or change in abundance of a biomolecule in sample may vary depending on the type of biomolecule. Suitable methods are well known in the art, and skilled artisan would be able to identify an appropriate method. Non-limiting examples of suitable methods to determine an amount of a biomolecule may include epitope binding agent-based methods (i.e. antibody- or aptamer-based methods, including ELISAs, radioimmunoassay, immunoblots, western blots), mass spectrometry based methods (for example, GC-MS, LC-MS, ESI-MS, ESI-tandem MS, MALDI-TOF), and array-based methods.

In some embodiments, the method for measuring the presence, absence or change in abundance of a biomolecule is an array. The array may be comprised of a substrate having disposed thereon at least one biomolecule. Several substrates suitable for the construction of arrays are known in the art. The substrate may be a material that may be modified to contain discrete individual sites appropriate for the attachment or association of the biomolecule and is amenable to at least one detection method. Alternatively, the substrate may be a material that may be modified for the bulk attachment or association of the biomolecule and is amenable to at least one detection method. Non-limiting examples of substrate materials include glass, modified or functionalized glass, plastics (including acrylics, polystyrene and copolymers of styrene and other materials, polypropylene, polyethylene, polybutylene, polyurethanes, TeflonJ, etc.), nylon or nitrocellulose, polysaccharides, nylon, resins, silica or silica-based materials including silicon and modified silicon, carbon, metals, inorganic glasses and plastics. In an exemplary embodiment, the substrates may allow optical detection without appreciably fluorescing.

A substrate may be planar, a substrate may be a well, i.e. a 1534-, 384-, or 96-well plate, or alternatively, a substrate may be a bead. Additionally, the substrate may be the inner surface of a tube for flow-through sample analysis to minimize sample volume. Similarly, the substrate may be flexible, such as a flexible foam, including closed cell foams made of particular plastics. Other suitable substrates are known in the art.

The biomolecule or biomolecules may be attached to the substrate in a wide variety of ways, as will be appreciated by those in the art. The biomolecule may either be synthesized first, with subsequent attachment to the substrate, or may be directly synthesized on the substrate. The substrate and the biomolecule may both be derivatized with chemical functional groups for subsequent attachment of the two. For example, the substrate may be derivatized with a chemical functional group including, but not limited to, amino groups, carboxyl groups, oxo groups or thiol groups. Using these functional groups, the biomolecule may be attached using functional groups on the biomolecule either directly or indirectly using linkers.

The biomolecule may also be attached to the substrate non-covalently. For example, a biotinylated biomolecule can be prepared, which may bind to surfaces covalently coated with streptavidin, resulting in attachment. Alternatively, a biomolecule or biomolecules may be synthesized on the surface using techniques such as photopolymerization and photolithography. Additional methods of attaching biomolecules to arrays and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, each of which is hereby incorporated by reference in its entirety).

In one embodiment, the biomolecule or biomolecules attached to the substrate are located at a spatially defined address of the array. Arrays may comprise from about 9 to about several hundred thousand addresses. In one embodiment, the array may be comprised of less than 10,000 addresses. In another alternative embodiment, the array may be comprised of at least 10,000 addresses. In yet another alternative embodiment, the array may be comprised of less than 5,000 addresses. In still another alternative embodiment, the array may be comprised of at least 5,000 addresses. In a further embodiment, the array may be comprised of less than 500 addresses. In yet a further embodiment, the array may be comprised of at least 500 addresses.

A biomolecule may be represented more than once on a given array. In other words, more than one address of an array may be comprised of the same biomolecule. In some embodiments, two, three, or more than three addresses of the array may be comprised of the same biomolecule. In certain embodiments, the array may comprise control biomolecules and/or control addresses. The controls may be internal controls, positive controls, negative controls, or background controls.

Furthermore, the biomolecules used for the array may be labeled. One skilled in the art understands that the type of label selected depends in part on how the array is being used. Suitable labels may include fluorescent labels, chromagraphic labels, chemi-luminescent labels, FRET labels, etc. Such labels are well known in the art.

TABLE 1
D. piger genes without identified mutations in the INSeq library that are presumably essential for D. piger survival in rich medium

| Gene_ID | Function | EC | KO | KEGG Pathway | KEGG Category |
|---|---|---|---|---|---|
| DpigGOR10047 | serine/threonine protein | EC2.7.11.1 | K08884 | Protein kinases | Enzyme Families |
| | kinase, bacterial | | | | |
| DpigGOR10093 | phosphate transport | EC3.6.3.27 | K02036 | Transporters; ABC | Membrane Transport |
| | system ATP-binding | | | transporters | |
| | protein | | | | |
| DpigGOR10097 | Unknown | Unknown | Unknown | Unknown | Unknown |
| DpigGOR10143 | D-alanine-D-alanine | EC6.3.2.4 | K01921 | D-Alanine | Metabolism of Other Amino |
| | ligase | | | metabolism; Peptidoglycan | Acids; Glycan Biosynthesis and |
| | | | | biosynthesis | Metabolism |
| DpigGOR10152 | Unknown | Unknown | Unknown | Unknown | Unknown |
| DpigGOR10155 | enoyl-(acyl-carrier | EC1.3.1.9 | K00208 | Fatty acid biosynthesis; Lipid | Lipid Metabolism |
| | protein) reductase I | | | biosynthesis proteins | |
| DpigGOR10184 | Unknown | Unknown | Unknown | Unknown | Unknown |
| DpigGOR10230 | ribose-phosphate | EC2.7.6.1 | K00948 | Pentose phosphate | Carbohydrate |
| | pyrophosphokinase | | | pathway; Purine metabolism | Metabolism; Nucleotide |
| | | | | | Metabolism |
| DpigGOR10233 | peptidyl-tRNA | EC3.1.1.29 | K01056 | Unclassified | Translation proteins |
| | hydrolase, PTH1 family | | | | |
| DpigGOR10286 | NOT DEFINED | EC3.4.24.— | K01417 | Unclassified | Others |
| DpigGOR10287 | tryptophanyl-tRNA | EC6.1.1.2 | K01867 | Tryptophan | Amino Acid |
| | synthetase | | | metabolism; Amino acid | Metabolism; Translation |
| | | | | related enzymes; Aminoacyl- | |
| | | | | tRNA biosynthesis | |
| DpigGOR10294 | large subunit ribosomal | NOT | K02909 | Ribosome | Translation |
| | protein L31 | DEFINED | | | |
| DpigGOR10319 | ubiquinone/menaquinone | EC2.1.1.— | K03183 | Ubiquinone and other | Metabolism of Cofactors and |
| | biosynthesis | | | terpenoid-quinone | Vitamins |
| | methyltransferase | | | biosynthesis | |
| DpigGOR10359 | glutamate-1- | EC5.4.3.8 | K01845 | Amino acid related | Amino Acid |
| | semialdehyde 2,1- | | | enzymes; Porphyrin and | Metabolism; Metabolism of |
| | aminomutase | | | chlorophyll metabolism | Cofactors and Vitamins |
| DpigGOR10374 | small subunit ribosomal | NOT | K02963 | Ribosome | Translation |
| | protein S18 | DEFINED | | | |
| DpigGOR10380 | aspartate-semialdehyde | EC1.2.1.11 | K00133 | Glycine, serine and threonine | Amino Acid Metabolism |
| | dehydrogenase | | | metabolism; Cysteine and | |
| | | | | methionine | |
| | | | | metabolism; Lysine | |
| | | | | biosynthesis | |
| DpigGOR10383 | dihydroorotate | NOT | K02823 | Unclassified | Energy metabolism |
| | dehydrogenase electron | DEFINED | | | |
| | transfer subunit | | | | |
| DpigGOR10400 | riboflavin kinase/FMN | EC2.7.1.26; | K11753 | Riboflavin metabolism | Metabolism of Cofactors and |
| | adenylyltransferase | EC2.7.7.2 | | | Vitamins |
| DpigGOR10412 | UDP-glucose 4- | EC5.1.3.2 | K01784 | Galactose metabolism; Amino | Carbohydrate Metabolism |
| | epimerase | | | sugar and nucleotide sugar | |
| | | | | metabolism | |
| DpigGOR10420 | glutamate racemase | EC5.1.1.3 | K01776 | D-Glutamine and D- | Metabolism of Other Amino Acids |
| | | | | glutamate metabolism | |
| DpigGOR10425 | Unknown | Unknown | Unknown | Unknown | Unknown |
| DpigGOR10435 | ArsR family | NOT | K03892 | Transcription factors | Transcription |
| | transcriptional regulator | DEFINED | | | |
| DpigGOR10436 | phosphopanto- | EC4.1.1.36; | K13038 | Pantothenate and CoA | Metabolism of Cofactors and |
| | thenoylcysteine | EC6.3.2.5 | | biosynthesis | Vitamins |
| | decarboxylase/ | | | | |
| DpigGOR10529 | ribulose-phosphate 3- | EC5.1.3.1 | K01783 | Pentose phosphate | Carbohydrate Metabolism; Energy |
| | epimerase | | | pathway; Pentose and | Metabolism |
| | | | | glucuronate | |
| | | | | interconversions; Carbon | |
| | | | | fixation in photosynthetic | |
| | | | | organisms | |
| DpigGOR10532 | Unknown | Unknown | Unknown | Unknown | Unknown |
| DpigGOR10548 | holo-(acyl-carrier | EC2.7.8.7 | K00997 | Pantothenate and CoA | Metabolism of Cofactors and |
| | protein) synthase | | | biosynthesis | Vitamins |
| DpigGOR10552 | hydroxymethylbilane | EC2.5.1.61 | K01749 | Porphyrin and chlorophyll | Metabolism of Cofactors and |
| | synthase | | | metabolism | Vitamins |
| DpigGOR10553 | Unknown | Unknown | Unknown | Unknown | Unknown |
| DpigGOR10555 | Unknown | Unknown | Unknown | Unknown | Unknown |
| DpigGOR10619 | Unknown | Unknown | Unknown | Unknown | Unknown |
| DpigGOR10620 | ribose 5-phosphate | EC5.3.1.6 | K01808 | Pentose phosphate | Carbohydrate Metabolism; Energy |
| | isomerase B | | | pathway; Carbon fixation in | Metabolism |
| | | | | photosynthetic organisms | |
| DpigGOR10635 | Unknown | Unknown | Unknown | Unknown | Unknown |
| DpigGOR10636 | branched-chain amino | EC2.6.1.42 | K00826 | Valine, leucine and isoleucine | Amino Acid |
| | acid aminotransferase | | | degradation; Valine, leucine | Metabolism; Metabolism of |
| | | | | and isoleucine | Cofactors and Vitamins |
| | | | | biosynthesis; Amino acid | |
| | | | | related | |
| | | | | enzymes; Pantothenate and | |
| | | | | CoA biosynthesis | |
| DpigGOR10648 | phosphatidylserine | EC2.7.8.8 | K00998 | Glycerophospholipid | Lipid Metabolism; Amino Acid |
| | synthase | | | metabolism; Glycine, serine | Metabolism |
| | | | | and threonine metabolism | |
| DpigGOR10649 | phosphatidylserine | EC4.1.1.65 | K01613 | Glycerophospholipid | Lipid Metabolism |
| | decarboxylase | | | metabolism | |
| DpigGOR10669 | alanine racemase | EC5.1.1.1 | K01775 | D-Alanine metabolism | Metabolism of Other Amino Acids |
| DpigGOR10681 | large subunit ribosomal | NOT | K02871 | Ribosome | Translation |
| | protein L13 | DEFINED | | | |
| DpigGOR10682 | small subunit ribosomal | NOT | K02996 | Ribosome | Translation |
| | protein S9 | DEFINED | | | |
| DpigGOR10722 | K06871 | NOT | K06871 | Unclassified | General function prediction only |
| | | DEFINED | | | |
| DpigGOR10737 | signal peptidase II | EC3.4.23.36 | K03101 | Peptidases; Protein export | Enzyme Families; Folding, Sorting |
| | | | | | and Degradation |
| DpigGOR10746 | pantetheine-phosphate | EC2.7.7.3 | K00954 | Pantothenate and CoA | Metabolism of Cofactors and |
| | adenylyltransferase | | | biosynthesis | Vitamins |
| DpigGOR10783 | K07121 | NOT | K07121 | Unclassified | General function prediction only |
| | | DEFINED | | | |
| DpigGOR10784 | K07121 | NOT | K07121 | Unclassified | General function prediction only |
| | | DEFINED | | | |
| DpigGOR10785 | aspartyl- | EC6.3.5.6; | K02435 | Aminoacyl-tRNA biosynthesis | Translation |
| | tRNA(Asn)/glutamyl- | EC6.3.5.7 | | | |
| | tRNA(Gln) | | | | |
| | amidotransferase | | | | |
| | subunit C | | | | |
| DpigGOR10823 | Unknown | Unknown | Unknown | Unknown | Unknown |
| DpigGOR10834 | GTP-binding protein | NOT | K03979 | Unclassified | General function prediction only |
| | | DEFINED | | | |
| DpigGOR10858 | Unknown | Unknown | Unknown | Unknown | Unknown |
| DpigGOR10867 | aspartyl-tRNA | EC6.1.1.12 | K01876 | Amino acid related | Amino Acid |
| | synthetase | | | enzymes; Aminoacyl-tRNA | Metabolism; Translation |
| | | | | biosynthesis | |
| DpigGOR10869 | methionyl-tRNA | EC2.1.2.9 | K00604 | One carbon pool by | Metabolism of Cofactors and |
| | formyltransferase | | | folate; Aminoacyl-tRNA | Vitamins; Translation |
| | | | | biosynthesis | |
| DpigGOR10870 | quinolinate synthase | EC2.5.1.72 | K03517 | Nicotinate and nicotinamide | Metabolism of Cofactors and |
| | | | | metabolism | Vitamins |
| DpigGOR10871 | L-aspartate oxidase | EC1.4.3.16 | K00278 | Alanine, aspartate and | Amino Acid |
| | | | | glutamate | Metabolism; Metabolism of |
| | | | | metabolism; Nicotinate and | Cofactors and Vitamins |
| | | | | nicotinamide metabolism | |
| DpigGOR10907 | HlyD family secretion | NOT | K02005 | Unclassified | Membrane and intracellular |
| | protein | DEFINED | | | structural molecules |
| DpigGOR10909 | Unknown | Unknown | Unknown | Unknown | Unknown |
| DpigGOR11040 | DNA (cytosine-5-)- | EC2.1.1.37 | K00558 | Cysteine and methionine | Amino Acid |
| | methyltransferase | | | metabolism; DNA replication | Metabolism; Replication and Repair |
| | | | | proteins; Chromosome | |
| DpigGOR11060 | GTP cyclohydrolase II/ | EC3.5.4.25; | K01497; | Riboflavin | Metabolism of Cofactors and |
| | 3,4-dihydroxy 2- | EC4.1.99.12 | K02858 | metabolism\|Riboflavin | Vitamins\|Metabolism of Cofactors |
| | butanone 4-phosphate | | | metabolism | and Vitamins |
| | synthase | | | | |
| DpigGOR11061 | GTP cyclohydrolase II/ | EC3.5.4.25; | K01497; | Riboflavin | Metabolism of Cofactors and |
| | 3,4-dihydroxy 2- | EC4.1.99.12 | K02858 | metabolism\|Riboflavin | Vitamins\|Metabolism of Cofactors |
| | butanone 4-phosphate | | | metabolism | and Vitamins |
| | synthase | | | | |
| DpigGOR11105 | Unknown | Unknown | Unknown | Unknown | Unknown |
| DpigGOR11122 | Unknown | Unknown | Unknown | Unknown | Unknown |
| DpigGOR11212 | Unknown | Unknown | Unknown | Unknown | Unknown |
| DpigGOR11227 | glycyl-tRNA synthetase | EC6.1.1.14 | K01878 | Aminoa cid related | Amino Acid |
| | alpha chain | | | enzymes; Aminoacyl-tRNA | Metabolism; Translation |
| | | | | biosynthesis | |
| DpigGOR11254 | IMP dehydrogenase | EC1.1.1.205 | K00088 | Purine metabolism; Drug | Nucleotide |
| | | | | metabolism - other enzymes | Metabolism; Xenobiotics |
| | | | | | Biodegradation and Metabolism |
| DpigGOR11255 | GMP synthase | EC6.3.5.2 | K01951 | Purine metabolism; Drug | Nucleotide |
| | (glutamine-hydrolysing) | | | metabolism - other | Metabolism; Xenobiotics |
| | | | | enzymes; Peptidases | Biodegradation and |
| | | | | | Metabolism; Enzyme Families |
| DpigGOR11259 | sec-independent protein | NOT | K03117 | Protein export; Bacterial | Folding, Sorting and |
| | translocase protein TatB | DEFINED | | secretion system; Secretion | Degradation; Membrane Transport |
| | | | | system | |
| DpigGOR11271 | glycerol-3-phosphate | EC2.3.1.15 | K08591 | Glycerolipid | Lipid Metabolism |
| | acyltransferase PlsY | | | metabolism; Glycerophospholipid | |
| | | | | metabolism; Lipid | |
| | | | | biosynthesis proteins | |
| DpigGOR11272 | exoribonuclease II | EC3.1.13.1 | K01147 | Unclassified | Translation proteins |
| DpigGOR11300 | thiamine biosynthesis | NOT | K03149 | Thiamine metabolism | Metabolism of Cofactors and |
| | ThiG | DEFINED | | | Vitamins |
| DpigGOR11306 | thiamine- | EC2.7.4.16 | K00946 | Thiamine metabolism | Metabolism of Cofactors and |
| | monophosphate kinase | | | | Vitamins |
| DpigGOR11310 | translation initiation | NOT | K02520 | Translation factors | Translation |
| | factor IF-3 | DEFINED | | | |
| DpigGOR11348 | preprotein translocase | NOT | K03074 | Protein export; Bacterial | Folding, Sorting and |
| | subunit SecF | DEFINED | | secretion system; Secretion | Degradation; Membrane Transport |
| | | | | system | |
| DpigGOR11350 | preprotein translocase | NOT | K03210 | Protein export; Bacterial | Folding, Sorting and |
| | subunit YajC | DEFINED | | secretion system; Secretion | Degradation; Membrane Transport |
| | | | | system | |
| DpigGOR11354 | Unknown | Unknown | Unknown | Unknown | Unknown |
| DpigGOR11360 | NAD + synthase | EC6.3.5.1 | K01950 | Nicotinate and nicotinamide | Metabolism of Cofactors and |
| | (glutamine-hydrolysing) | | | metabolism | Vitamins |
| DpigGOR11361 | 3-octaprenyl-4- | EC4.1.1.— | K03182 | Ubiquinone and other | Metabolism of Cofactors and |
| | hydroxybenzoate | | | terpenoid-quinone | Vitamins |
| | carboxy-lyase UbiD | | | biosynthesis | |
| DpigGOR11402 | nicotinate-nucleotide | EC2.7.7.18 | K00969 | Nicotinate and nicotinamide | Metabolism of Cofactors and |
| | adenylyltransferase | | | metabolism | Vitamins |
| DpigGOR11415 | 3R-hydroxymyristoyl | EC4.2.1.— | K02372 | Fatty acid biosynthesis; Lipid | Lipid Metabolism |
| | ACP dehydrase | | | biosynthesis proteins | |
| DpigGOR11420 | lipoprotein-releasing | EC3.6.3.— | K09810 | Transporters; ABC | Membrane Transport |
| | system ATP-binding | | | transporters | |
| | protein | | | | |
| DpigGOR11439 | fused signal recognition | NOT | K03110 | Protein export; Bacterial | Folding, Sorting and |
| | particle receptor | DEFINED | | secretion system; Secretion | Degradation; Membrane Transport |
| | | | | system | |
| DpigGOR11441 | small subunit ribosomal | NOT | K02946 | Ribosome | Translation |
| | protein S10 | DEFINED | | | |
| DpigGOR11442 | large subunit ribosomal | NOT | K02906 | Ribosome | Translation |
| | protein L3 | DEFINED | | | |
| DpigGOR11443 | large subunit ribosomal | NOT | K02926 | Ribosome | Translation |
| | protein L4 | DEFINED | | | |
| DpigGOR11444 | large subunit ribosomal | NOT | K02892 | Ribosome | Translation |
| | protein L23 | DEFINED | | | |
| DpigGOR11445 | large subunit ribosomal | NOT | K02886 | Ribosome | Translation |
| | protein L2 | DEFINED | | | |
| DpigGOR11447 | large subunit ribosomal | NOT | K02890 | Ribosome | Translation |
| | protein L22 | DEFINED | | | |
| DpigGOR11448 | small subunit ribosomal | NOT | K02982 | Ribosome | Translation |
| | protein S3 | DEFINED | | | |
| DpigGOR11452 | small subunit ribosomal | NOT | K02961 | Ribosome | Translation |
| | protein S17 | DEFINED | | | |
| DpigGOR11453 | large subunit ribosomal | NOT | K02874 | Ribosome | Translation |
| | protein L14 | DEFINED | | | |
| DpigGOR11454 | large subunit ribosomal | NOT | K02895 | Ribosome | Translation |
| | protein L24 | DEFINED | | | |
| DpigGOR11455 | large subunit ribosomal | NOT | K02931 | Ribosome | Translation |
| | protein L5 | DEFINED | | | |
| DpigGOR11456 | small subunit ribosomal | NOT | K02954 | Ribosome | Translation |
| | protein S14 | DEFINED | | | |
| DpigGOR11458 | small subunit ribosomal | NOT | K02994 | Ribosome | Translation |
| | protein S8 | DEFINED | | | |
| DpigGOR11459 | large subunit ribosomal | NOT | K02933 | Ribosome | Translation |
| | protein L6 | DEFINED | | | |
| DpigGOR11460 | large subunit ribosomal | NOT | K02881 | Ribosome | Translation |
| | protein L18 | DEFINED | | | |
| DpigGOR11461 | small subunit ribosomal | NOT | K02988 | Ribosome | Translation |
| | protein S5 | DEFINED | | | |
| DpigGOR11463 | large subunit ribosomal | NOT | K02876 | Ribosome | Translation |
| | protein L15 | DEFINED | | | |
| DpigGOR11466 | small subunit ribosomal | NOT | K02952 | Ribosome | Translation |
| | protein S13 | DEFINED | | | |
| DpigGOR11468 | small subunit ribosomal | NOT | K02986 | Ribosome | Translation |
| | protein S4 | DEFINED | | | |
| DpigGOR11469 | DNA-directed RNA | EC2.7.7.6 | K03040 | Purine | Nucleotide |
| | polymerase subunit | | | metabolism; Pyrimidine | Metabolism; Transcription; |
| | alpha | | | metabolism; RNA | Replication and Repair |
| | | | | polymerase; DNA repair and | |
| | | | | recombination proteins | |
| DpigGOR11522 | biopolymer transport | NOT | K03562 | Unclassified | Cell motility and secretion |
| | protein TolQ | DEFINED | | | |
| DpigGOR11523 | biopolymer transport | NOT | K03559 | Unclassified | Cell motility and secretion |
| | protein ExbD | DEFINED | | | |
| DpigGOR11524 | colicin import | NOT | K03646 | Unclassified | Pores ion channels |
| | membrane protein | DEFINED | | | |
| DpigGOR11532 | Unknown | Unknown | Unknown | Unknown | Unknown |
| DpigGOR11535 | Unknown | Unknown | Unknown | Unknown | Unknown |
| DpigGOR11617 | Cu2+-exporting ATPase | EC3.6.3.4 | K01533 | Unclassified | Energy metabolism |
| DpigGOR11690 | hypothetical protein | NOT | K09791 | Unclassified | Function unknown |
| | | DEFINED | | | |
| DpigGOR11727 | Unknown | Unknown | Unknown | Unknown | Unknown |
| DpigGOR11776 | Unknown | Unknown | Unknown | Unknown | Unknown |
| DpigGOR11830 | Unknown | Unknown | Unknown | Unknown | Unknown |
| DpigGOR11853 | Unknown | Unknown | Unknown | Unknown | Unknown |
| DpigGOR11855 | NOT DEFINED | EC2.—.—.— | K01043 | Unclassified | Others |
| DpigGOR11856 | Unknown | Unknown | Unknown | Unknown | Unknown |
| DpigGOR11868 | small subunit ribosomal | NOT | K02950 | Ribosome | Translation |
| | protein S12 | DEFINED | | | |
| DpigGOR11882 | MraZ protein | NOT | K03925 | Unclassified | Function unknown |
| | | DEFINED | | | |
| DpigGOR11883 | S-adenosyl- | EC2.1.1.— | K03438 | Unclassified | Membrane and intracellular |
| | methyltransferase | | | | structural molecules |
| DpigGOR11884 | Unknown | Unknown | Unknown | Unknown | Unknown |
| DpigGOR11885 | cell division protein FtsI | EC2.4.1.129 | K03587 | Peptidoglycan | Glycan Biosynthesis and |
| | (penicillin-binding | | | biosynthesis; Chromosome | Metabolism; Replication and Repair |
| | protein 3) | | | | |
| DpigGOR11886 | UDP-N- | EC6.3.2.13 | K01928 | Lysine | Amino Acid Metabolism; Glycan |
| | acetylmuramoylalanyl- | | | biosynthesis; Peptidoglycan | Biosynthesis and Metabolism |
| | D-glutamate--2,6- | | | biosynthesis | |
| | diaminopimelate ligase | | | | |
| DpigGOR11887 | UDP-N- | EC6.3.2.10 | K01929 | Lysine | Amino Acid Metabolism; Glycan |
| | acetylmuramoylalanyl- | | | biosynthesis; Peptidoglycan | Biosynthesis and Metabolism |
| | D-glutamyl-2,6- | | | biosynthesis | |
| | diaminopimelate--D- | | | | |
| | alanyl- | | | | |
| DpigGOR11890 | cell division protein | NOT | K03588 | Chromosome; Cell cycle - | Replication and Repair; Cell Growth |
| | FtsW | DEFINED | | Caulobacter | and Death |
| DpigGOR11891 | UDP-N- | EC2.4.1.227 | K02563 | Peptidoglycan | Glycan Biosynthesis and |
| | acetylglucosamine--N- | | | biosynthesis; Cell cycle - | Metabolism; Cell Growth and Death |
| | acetylmuramyl- | | | Caulobacter | |
| | (pentapeptide) | | | | |
| DpigGOR11892 | UDP-N-acetylmuramate-- | EC6.3.2.8 | K01924 | D-Glutamine and D- | Metabolism of Other Amino |
| | alanine ligase | | | glutamate | Acids; Glycan Biosynthesis and |
| | | | | metabolism; Peptidoglycan | Metabolism |
| | | | | biosynthesis | |
| DpigGOR11893 | UDP-N-acetylmuramate | EC1.1.1.158 | K00075 | Amino sugar and nucleotide | Carbohydrate Metabolism; Glycan |
| | dehydrogenase | | | sugar | Biosynthesis and Metabolism |
| | | | | metabolism; Peptidoglycan | |
| | | | | biosynthesis | |
| DpigGOR11900 | 1-deoxy-D-xylulose-5- | EC1.1.1.267 | K00099 | Terpenoid backbone | Metabolism of Terpenoids and |
| | phosphate | | | biosynthesis | Polyketides |
| | reductoisomerase | | | | |
| DpigGOR11901 | phosphatidate | EC2.7.7.41 | K00981 | Glycerophospholipid | Lipid Metabolism; Signal |
| | cytidylyltransferase | | | metabolism; Phosphatidylinositol | Transduction |
| | | | | signaling system | |
| DpigGOR11902 | undecaprenyl | EC2.5.1.31 | K00806 | Prenyltransferases; Terpenoid | Metabolism of Terpenoids and |
| | diphosphate synthase | | | backbone biosynthesis | Polyketides |
| DpigGOR11903 | ribosome recycling | NOT | K02838 | Translation factors | Translation |
| | factor | DEFINED | | | |
| DpigGOR11942 | K07164 | NOT | K07164 | Unclassified | General function prediction only |
| | | DEFINED | | | |
| DpigGOR12007 | Unknown | Unknown | Unknown | Unknown | Unknown |
| DpigGOR12060 | signal recognition | NOT | K03106 | Protein export; Bacterial | Folding, Sorting and |
| | particle subunit SRP54 | DEFINED | | secretion system; Secretion | Degradation; Membrane Transport |
| | | | | system | |
| DpigGOR12061 | small subunit ribosomal | NOT | K02959 | Ribosome | Translation |
| | protein S16 | DEFINED | | | |
| DpigGOR12075 | large subunit ribosomal | NOT | K02884 | Ribosome | Translation |
| | protein L19 | DEFINED | | | |
| DpigGOR12082 | phosphoglucosamine | EC5.4.2.10 | K03431 | Amino sugar and nucleotide | Carbohydrate Metabolism |
| | mutase | | | sugar metabolism | |
| DpigGOR12083 | UTP--glucose-1- | EC2.7.7.9 | K00963 | Pentose and glucuronate | Carbohydrate Metabolism |
| | phosphate | | | interconversions; Galactose | |
| | uridylyltransferase | | | metabolism; Starch and | |
| | | | | sucrose metabolism; Amino | |
| | | | | sugar and nucleotide sugar | |
| | | | | metabolism | |
| DpigGOR12085 | chromosomal | NOT | K02313 | DNA replication | Replication and Repair; Signal |
| | replication initiator | DEFINED | | proteins; Chromosome; Two- | Transduction; Cell Growth and |
| | protein | | | component system; Cell cycle - | Death |
| | | | | Caulobacter | |
| DpigGOR12099 | ceramide | EC2.4.1.80 | K00720 | Sphingolipid | Lipid Metabolism; Glycan |
| | glucosyltransferase | | | metabolism; Glycosyltransferases | Biosynthesis and Metabolism |
| DpigGOR12100 | Unknown | Unknown | Unknown | Unknown | Unknown |
| DpigGOR12102 | Unknown | Unknown | Unknown | Unknown | Unknown |
| DpigGOR12139 | Unknown | Unknown | Unknown | Unknown | Unknown |
| DpigGOR12160 | Unknown | Unknown | Unknown | Unknown | Unknown |
| DpigGOR12210 | CDP-diacylglycerol-- | EC2.7.8.5 | K00995 | Glycerophospholipid | Lipid Metabolism |
| | glycerol-3-phosphate 3- | | | metabolism | |
| | phosphatidyltransferase | | | | |
| DpigGOR12211 | Unknown | Unknown | Unknown | Unknown | Unknown |
| DpigGOR12212 | cell division protein FtsB | NOT | K05589 | Chromosome | Replication and Repair |
| | | DEFINED | | | |
| DpigGOR12213 | Unknown | Unknown | Unknown | Unknown | Unknown |
| DpigGOR12217 | thioredoxin 1 | NOT | K03671 | Chaperones and folding | Folding, Sorting and Degradation |
| | | DEFINED | | catalysts | |
| DpigGOR12218 | thioredoxin reductase | EC1.8.1.9 | K00384 | Pyrimidine metabolism | Nucleotide Metabolism |
| | (NADPH) | | | | |
| DpigGOR12221 | GTP-binding protein | NOT | K03978 | Unclassified | General function prediction only |
| | | DEFINED | | | |
| DpigGOR12224 | outer membrane | NOT | K03634 | Unclassified | Membrane and intracellular |
| | lipoprotein carrier | DEFINED | | | structural molecules |
| | protein | | | | |
| DpigGOR12245 | (E)-4-hydroxy-3- | EC1.17.7.1 | K03526 | Terpenoid backbone | Metabolism of Terpenoids and |
| | methylbut-2-enyl- | | | biosynthesis | Polyketides |
| | diphosphate synthase | | | | |
| DpigGOR12249 | exodeoxyribonuclease | EC3.1.11.6 | K03602 | Mismatch repair; DNA repair | Replication and Repair |
| | VII small subunit | | | and recombination proteins | |
| DpigGOR12250 | geranylgeranyl | NOT | K13789 | Prenyltransferases; Terpenoid | Metabolism of Terpenoids and |
| | diphosphate synthase, | DEFINED | | backbone biosynthesis | Polyketides |
| | type II | | | | |
| DpigGOR12251 | 1-deoxy-D-xylulose-5- | EC2.2.1.7 | K01662 | Terpenoid backbone | Metabolism of Terpenoids and |
| | phosphate synthase | | | biosynthesis | Polyketides |
| DpigGOR12254 | glutamyl-tRNA | EC1.2.1.70 | K02492 | Porphyrin and chlorophyll | Metabolism of Cofactors and |
| | reductase | | | metabolism | Vitamins |
| DpigGOR12255 | Unknown | Unknown | Unknown | Unknown | Unknown |
| DpigGOR12256 | precorrin-2 | EC1.3.1.76; | K02304 | Porphyrin and chlorophyll | Metabolism of Cofactors and |
| | dehydrogenase/ | EC4.99.1.4 | | metabolism | Vitamins |
| | sirohydrochlorin | | | | |
| | ferrochelatase | | | | |
| DpigGOR12258 | Unknown | Unknown | Unknown | Unknown | Unknown |
| DpigGOR12288 | uroporphyrinogen III | NOT | K13542 | Porphyrin and chlorophyll | Metabolism of Cofactors and |
| | methyltransferase/ | DEFINED | | metabolism | Vitamins |
| | synthase | | | | |
| DpigGOR12324 | hypothetical protein | NOT | K09141 | Unclassified | Function unknown |
| | | DEFINED | | | |
| DpigGOR12354 | Unknown | Unknown | Unknown | Unknown | Unknown |
| DpigGOR12360 | hypothetical protein | NOT | K09117 | Unclassified | Function unknown |
| | | DEFINED | | | |
| DpigGOR12362 | DNA primase | EC2.7.7.— | K02316 | DNA replication; DNA | Replication and Repair |
| | | | | replication proteins | |
| DpigGOR12409 | guanylate kinase | EC2.7.4.8 | K00942 | Purine metabolism | Nucleotide Metabolism |
| DpigGOR12425 | acyl carrier protein | NOT | K02078 | Unclassified | Lipid metabolism |
| | | DEFINED | | | |
| DpigGOR12426 | 3-oxoacyl-(acyl-carrier- | EC2.3.1.179 | K09458 | Fatty acid biosynthesis; Lipid | Lipid Metabolism |
| | protein) synthase II | | | biosynthesis proteins | |
| DpigGOR12430 | diaminohydroxyphospho- | NOT | K11752 | Riboflavin metabolism | Metabolism of Cofactors and |
| | ribosylaminopyrimidine | DEFINED | | | Vitamins |
| | deaminase/ | | | | |
| DpigGOR12431 | riboflavin synthase | EC2.5.1.9 | K00793 | Riboflavin metabolism | Metabolism of Cofactors and |
| | alpha chain | | | | Vitamins |
| DpigGOR12432 | riboflavin synthase beta | EC2.5.1.— | K00794 | Riboflavin metabolism | Metabolism of Cofactors and |
| | chain | | | | Vitamins |
| DpigGOR12433 | N utilization substance | NOT | K03625 | Unclassified | Transcription related proteins |
| | protein B | DEFINED | | | |
| DpigGOR12435 | DNA polymerase III | EC2.7.7.7 | K02340 | Purine | Nucleotide Metabolism; Replication |
| | subunit delta | | | metabolism; Pyrimidine | and Repair |
| | | | | metabolism; DNA | |
| | | | | replication; DNA replication | |
| | | | | proteins; Mismatch | |
| | | | | repair; Homologous | |
| | | | | recombination; DNA repair | |
| | | | | and recombination proteins | |
| DpigGOR12438 | methyltransferase | EC2.1.1.— | K02493 | Unclassified | Translation proteins |
| DpigGOR12459 | elongation factor EF-Tu | EC3.6.5.3 | K02358 | Translation factors; Plant- | Translation; Environmental |
| | | | | pathogen interaction | Adaptation |
| DpigGOR12461 | preprotein translocase | NOT | K03073 | Protein export; Bacterial | Folding, Sorting and |
| | subunit SecE | DEFINED | | secretion system; Secretion | Degradation; Membrane Transport |
| | | | | system | |
| DpigGOR12463 | large subunit ribosomal | NOT | K02867 | Ribosome | Translation |
| | protein L11 | DEFINED | | | |
| DpigGOR12465 | large subunit ribosomal | NOT | K02864 | Ribosome | Translation |
| | protein L10 | DEFINED | | | |
| DpigGOR12466 | large subunit ribosomal | NOT | K02935 | Ribosome | Translation |
| | protein L7/L12 | DEFINED | | | |
| DpigGOR12470 | Unknown | Unknown | Unknown | Unknown | Unknown |

TABLE 2
D. piger fitness determinants that exhibit diet-sensitivity

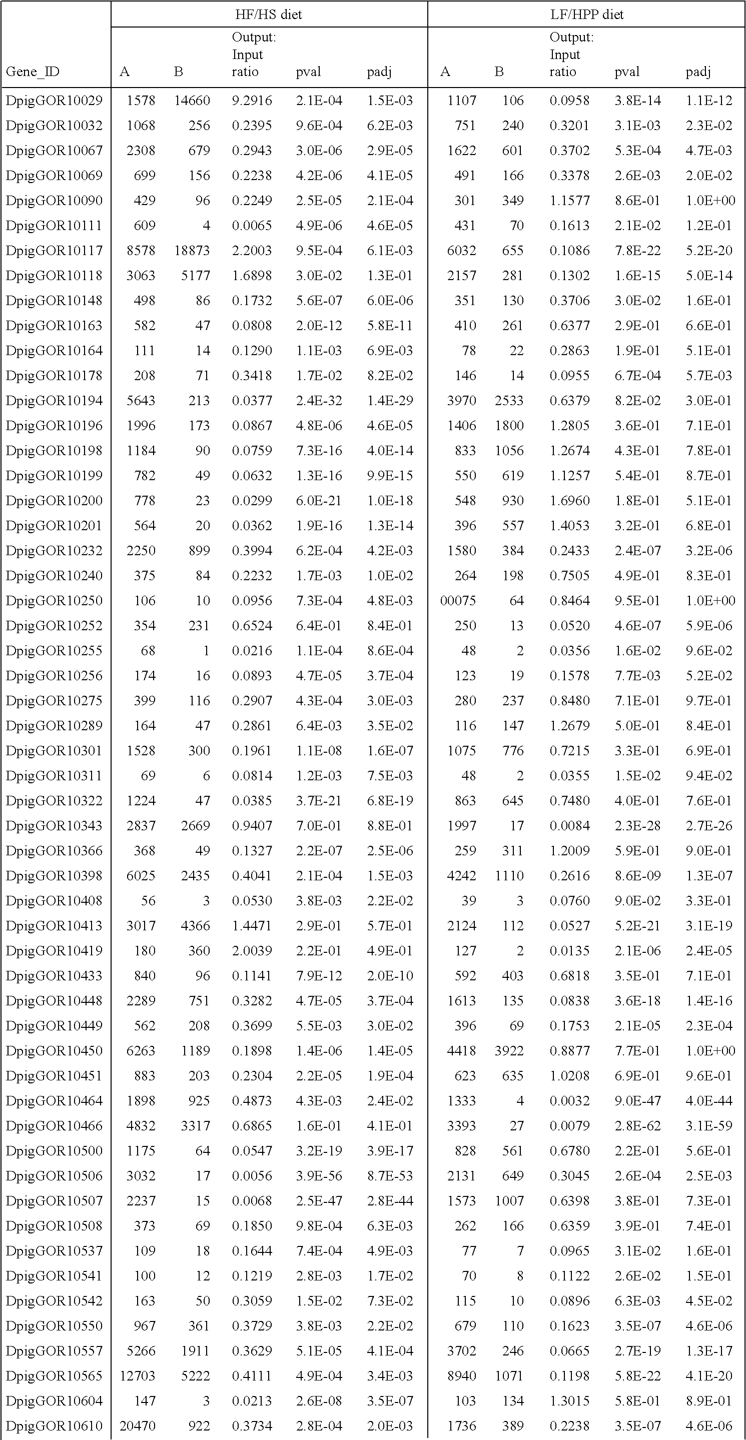

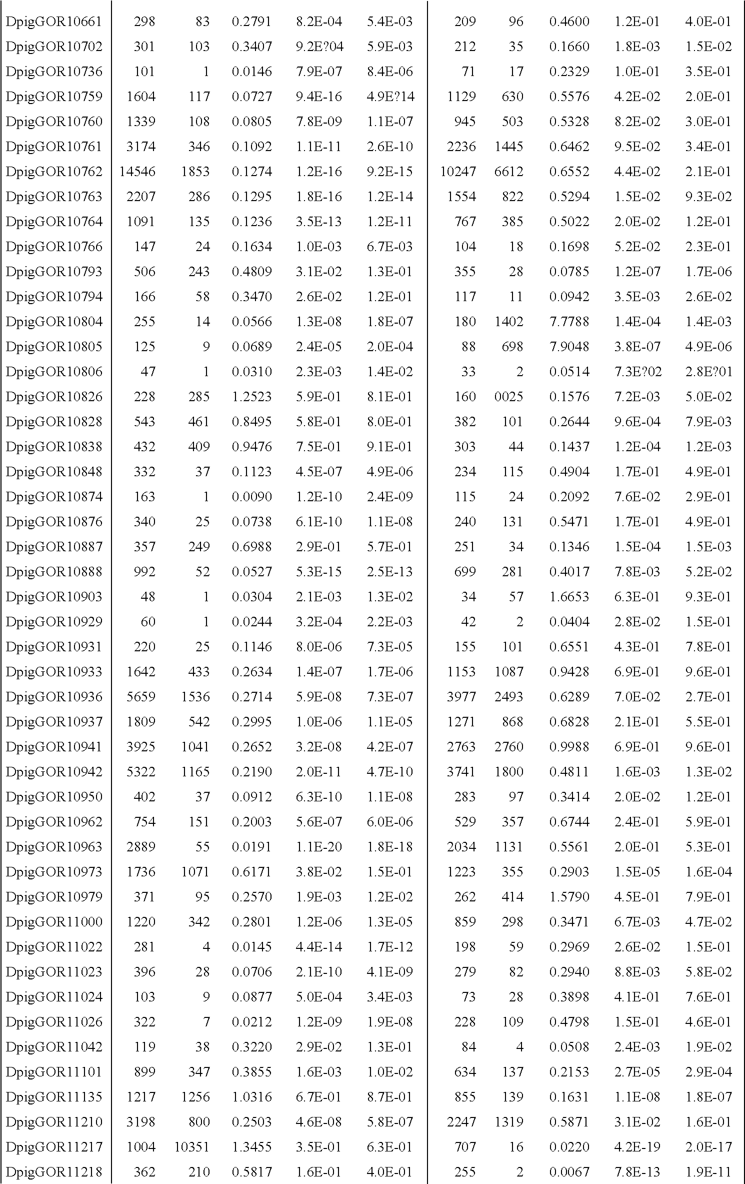

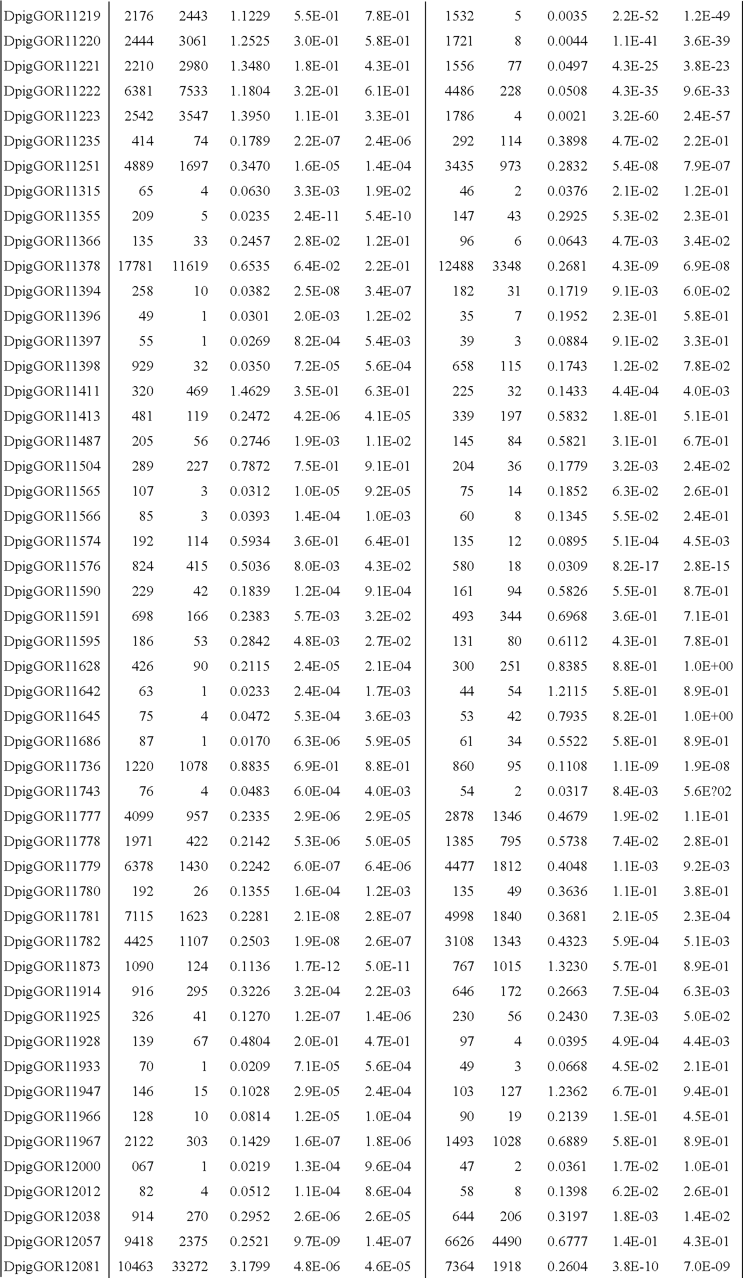

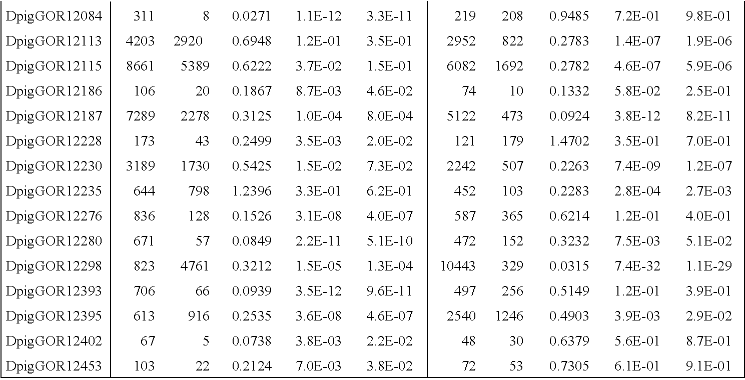

A = Normalized input reads (mean)
B = Normalized output reads (mean)
Highlighted rows indicate significant difference relative to input (padj < 0.005; output: input ratio < 0.3)

TABLE 3
D. piger fitness determinants that exhibit in vivo specificity

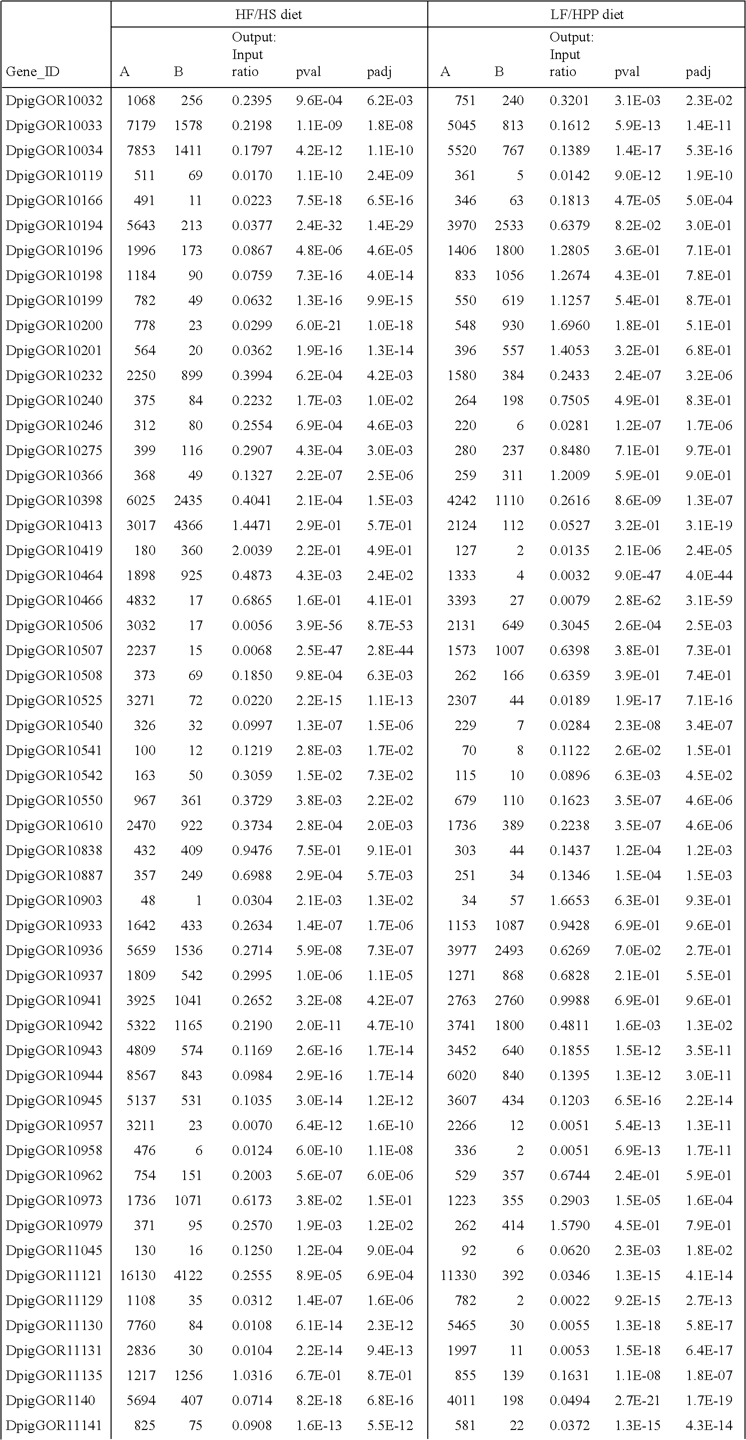

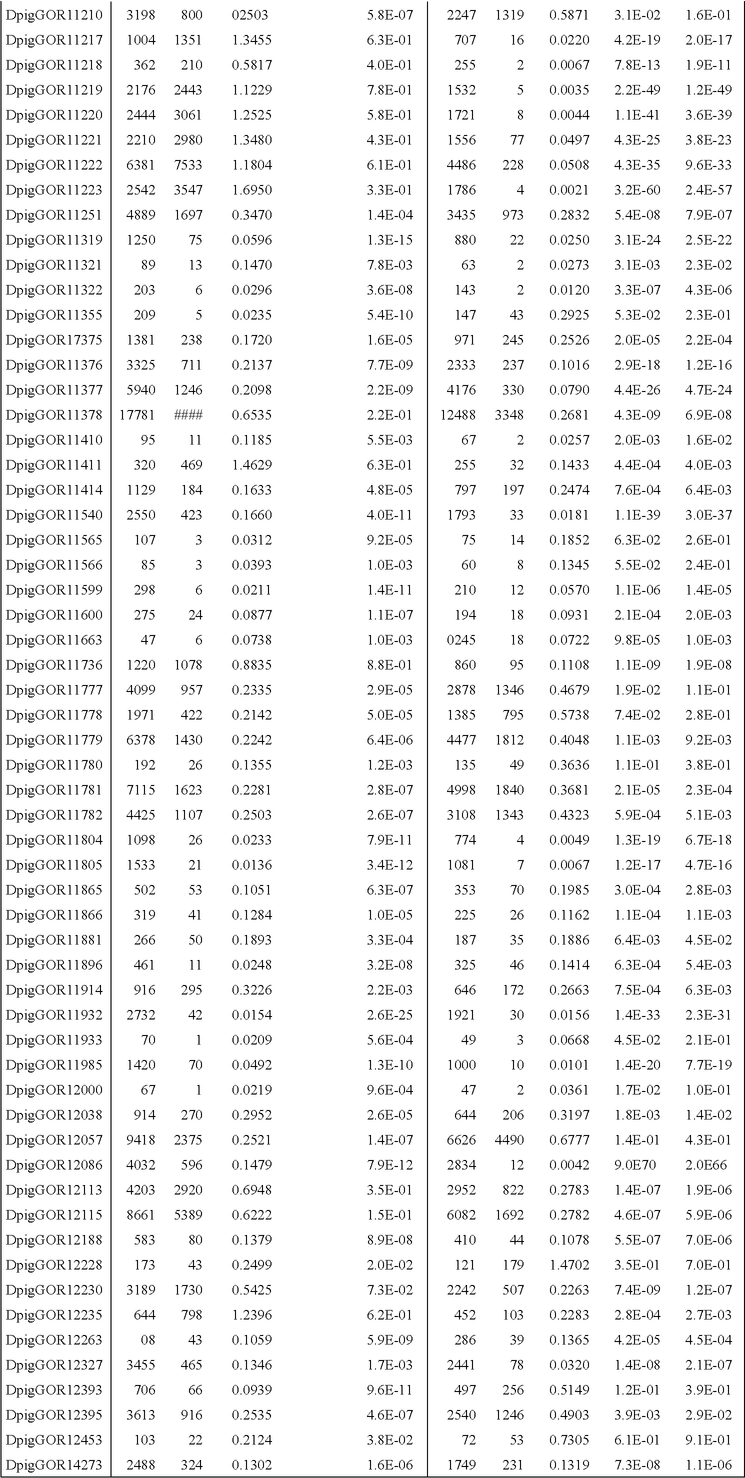

A = Normalized input reads (mean)
B = Normalized output reads (mean)
Highlighted rows indicate significant differences relative to input (padj < 0.005; output: input ratio < 0.3). Analysis of fecal samples.

TABLE 4
Effect of D. piger on the microbial community metatranscriptome

| | Fold Change (8-member | | | |
|---|---|---|---|---|
| | community plus D. piger vs. 8 | | | |
| EC number | member community) | p-value | ppde | Description |
| EC4.1.1.37 | 4.2 | 4.8E−04 | 0.96 | uroporphyrinogen decarboxylase |
| EC3.2.1.52 | −1.9 | 4.6E−04 | 0.96 | N-acetyl-β-glucosaminidase subunit |
| EC4.2.2.17 | −2.1 | 6.1E−04 | 0.95 | inulin fructotransferase (DFA-I-forming) |
| EC3.2.1.139 | −2.3 | 6.3E−04 | 0.95 | α-glucosiduronase |
| EC3.1.6.6 | −2.8 | 6.6E−05 | 0.98 | choline sulfatase |
| EC4.2.2.20 | −2.8 | 1.4E−05 | 0.99 | chondroitin ABC endo-lyase |
| EC4.2.2.21 | −2.8 | 1.4E−05 | 0.99 | chondroitin sulfate ABC lyase II |
| EC1.1.1.37 | −3.0 | 2.4E−04 | 0.97 | malate dehydrogenase |
| EC3.2.1.14 | −3.8 | 4.3E−05 | 0.99 | glycoside hydrolase Family 18. |
| EC2.3.2.2 | −7.0 | 8.4E−06 | 1.00 | γ-glutamyl transpeptidase (GGT) |

TABLE 5
Effects of the presence or absence of D. piger on mouse gene expression in the proximal colon

| | | Fold Change (8-member community |
|---|---|---|
| Gene name | Description | plus D. piger vs 8-member community |
| PTPN5 | protein tyrosine phosphatase, non-receptor type 5 (striatum-enriched) | 3.5 |
| MMP7 | matrix metallopeptidase 7 (matrilysin, uterine) | 2.5 |
| ARSJ | arylsulfatase family, member J | 2.2 |
| CDKL1 | cyclin-dependent kinase-like 1 (CDC2-related kinase) | 2.2 |
| DPP10 | dipeptidyl-peptidase 10 (non-functional) | 2.0 |
| PRKG2 | protein kinase, cGMP-dependent, type II | 2.0 |
| GRIN3A | glutamate receptor, ionotropic, N-methyl-D-aspartate 3A | 2.0 |
| BCL3 | B-cell CLL/lymphoma 3 | −2.0 |
| FSCN1 | fascin homolog 1, actin-bundling protein (Strongylocentrotus purpuratus) | −2.1 |
| OASL | 2′-5′-oligoadenylate synthetase-like | −2.2 |
| DHRS9 | dehydrogenase/reductase (SDR family) member 9 | −2.2 |
| ITGAL | integrin, alpha L (antigen CD11A (p180), lymphocyte function- | −2.2 |
| | associated antigen 1; alpha polypeptide) | |
| CLDN4 | claudin 4 | −2.2 |
| AQP8 | aquaporin 8 | −2.2 |
| EGLN3 | egl nine homolog 3 (C. elegans) | −2.3 |
| DUOXA1 | dual oxidase maturation factor 1 | −2.5 |
| GSDMC | gasdermin C | −2.5 |
| IGJ | immunoglobulin J polypeptide, linker protein for immunoglobulin alpha | −2.5 |
| | and mu polypeptides | |
| MFSD2A | major facilitator superfamily domain containing 2A | −2.6 |
| BHLHA15 | basic helix-loop-helix family, member a15 | −2.7 |
| ETV4 | ets variant 4 | −2.8 |
| ABCG8 | ATP-binding cassette, sub-family G (WHITE), member 8 | −2.9 |
| MZB1 | marginal zone B and B1 cell-specific protein | −2.9 |
| TNFRSF17 | tumor necrosis factor receptor superfamily, member 17 | −3.0 |
| CD79A | CD79a molecule, immunoglobulin-associated alpha | −3.0 |
| GDF15 | growth differentiation factor 15 | −3.0 |
| Sprr1a | small proline-rich protein 1A | −3.2 |
| DUOXA2 | dual oxidase maturation factor 2 | −3.3 |
| Xlr3c | X-linked lymphocyte-regulated 3C | −3.3 |
| (includes | | |
| others) | | |
| SLC37A2 | solute carrier family 37 (glycerol-3-phosphate transporter), member 2 | −3.6 |
| CPS1 | carbamoyl-phosphate synthase 1, mitochondrial | −3.9 |
| ABCG5 | ATP-binding cassette, sub-family G (WHITE), member 5 | −4.6 |
| COCH | coagulation factor C homolog, cochlin (Limulus polyphemus) | −4.6 |
| Igkv6-14 | immunoglobulin kappa variable 6-14 | −4.8 |
| IGHM | immunoglobulin heavy constant mu | −5.0 |
| IGHA1 | immunoglobulin heavy constant alpha 1 | −5.6 |
| Ighg | Immunoglobulin heavy chain (gamma polypeptide) | −8.6 |
| Ighg2c | immunoglobulin heavy constant gamma 2C | −11.8 |
| Igh-VS107 | immunoglobulin heavy chain (S107 family) | −13.5 |

Genes with significant changes are shown;
threshold cut-off p < 0.005; fold change >2 or <−2

TABLE 6
Abundancea of acylcarnitines, TCA cycle intermediates and glutathione in livers from mice
colonized with the 8-member community and the 8-member community plus D. piger

| | | | | | | | | | Fold- | P- |
|---|---|---|---|---|---|---|---|---|---|---|
| metabolite | 8-member community plus D. piger | | | | | 8-member community | | | change^{b} | value^{c} |
| L-Acetylcarnitine | 8.1E+07 | 6.2E+07 | 6.4E+07 | 6.9E+07 | 7.7E+07 | 1.0E+08 | 1.0E+08 | 8.0E+07 | 0.75 | 0.0152 |
| Propionylcarnitine | 1.2E+07 | 1.5E+07 | 1.3E+07 | 1.2E+07 | 1.2E+07 | 2.1E+07 | 1.6E+07 | 1.5E+07 | 0.75 | 0.0340 |
| butyryl-carnitine | 1.1E+07 | 9.9E+06 | 8.9E+06 | 1.2E+07 | 1.4E+07 | 2.0E+07 | 1.7E+07 | 1.2E+07 | 0.69 | 0.0485 |
| pimelylcarnitine | 5.1E+07 | 1.0E+08 | 1.3E+08 | 4.4E+07 | 8.2E+07 | 3.9E+07 | 3.6E+07 | 3.2E+07 | 2.29 | 0.0736 |
| succinic acid | 1.3E+07 | 8.0E+06 | 7.9E+06 | 8.1E+06 | 8.2E+06 | 8.3E+06 | 9.6E+06 | 8.0E+06 | 1.04 | 0.8019 |
| fumarate | 1.9E+06 | 5.7E+05 | 5.7E+05 | 8.8E+05 | 6.5E+05 | 6.8E+05 | 9.2E+05 | 9.1E+05 | 1.09 | 0.8339 |
| glutathione | 3.5E+05 | 8.1E+04 | 8.8E+04 | 4.6E+05 | 1.9E+05 | 1.4E+06 | 1.7E+06 | 1.2E+06 | 0.16 | 0.0001 |
| (reduced) | | | | | | | | | | |
| glutathione | 4.3E+06 | 2.5E+06 | 4.4E+06 | 3.4E+06 | 3.7E+06 | 4.8E+06 | 3.6E+06 | 4.6E+06 | 0.84 | 0.2322 |
| (oxidized) | | | | | | | | | | |

a, raw intensity values from UPLC-MS
^{b}fold-change = 8-member community plus D. piger/8-member community
^{c}two-tailed t-test

TABLE 7
Media used for growth of bacteria

| | | |
|---|---|---|
| Component | quantity/L | Comments |

MegaMedia 2.0- medium used for matings
and D. piger mutant library selection

| | | | |
|---|---|---|---|
| Tryptone Peptone | 10 | g | |
| Yeast Extract | 5 | g | |
| D-glucose | 2 | g | |
| L-Cysteine HCl | 0.5 | g | |
| Na₂S0₄ | 2 | g | |
| Malate | 0.5 | g | |
| KH₂PO₄ | 100 | ml | 1M stock solution, pH 7.2 |
| Vitamin K (menadione) | 1 | ml | 1 mg/ml in 100% ethanol |
| | | | stock solution |
| MgSO₄•7H₂0 | 0.02 | g | |
| NaHCO₃ | 0.4 | g | |
| NaCl | 0.08 | g | |
| CaCl₂ | 1 | ml | 0.8 g/100 ml dH₂0 stock |
| | | | solution |
| FeSO₄ | 1 | ml | 40 mg/100 ml dH₂0 stock |
| | | | solution |
| Resazurin | 4 | ml | 25 mg resazurin/100 ml of |
| | | | dH₂0 stock solution |
| Histidine Hematin | 1 | ml | 1.2 mg hematin/ml in 0.2M |
| | | | histidine (pH 8.0) stock |
| | | | solution |
| Na Acetate | 1 | g | |
| Meat Extract | 5 | g | |
| ATCC Vitamin Mix | 10 | ml | |
| ATCC Trace Mineral | 10 | ml | |
| Mix | | | |
| Noble Agar | 12 | g | |

SRB641- medium used for routine
growth of D. piger GOR1

| | | |
|---|---|---|
| NH₄Cl | 1 | g |
| Na₂SO₄ | 2 | g |
| Na₂S₂0₃•5H₂0 | 1 | g |
| MgSO₄•7H₂0 | 1 | g |
| CaCl₂•2H₂0 | 0.1 | g |
| KH₂PO₄ | 0.5 | g |
| Yeast extract | 1 | g |
| Resazurin | 0.5 | ml |
| Cysteine | 0.6 | g |
| DTT | 0.6 | g |
| NaHCO₃ | 1 | g |
| Pyruvic acid | 3 | g |
| Malic acid | 3 | g |
| ATCC Trace Mineral | 10 | ml |
| Mix | | |
| ATCC Vitamin Mix | 10 | ml |
| adjust pH to 7.2 and | | |
| filter sterilized | | |

SRB medium supplemented with 20 amino
acid used for INSeq library selection

| | | |
|---|---|---|
| Na₂SO₄ | 2 | g |
| MgSO₄•7H₂0 | 1 | g |
| CaCl₂•2H₂0 | 0.1 | g |
| KH₂PO₄ | 0.5 | g |
| Resazurin | 0.5 | ml |
| Alanine | 2 | g |
| Asparagine | 2 | g |
| Arginine HCl | 2 | g |
| Aspartic acid | 2 | g |
| Cysteine HCl | 2.89 | g |
| Glutamine | 2 | g |
| Glutamic acid | 2 | g |
| Glycine | 2 | g |
| Histidine HCl | 2.42 | g |
| Isoleucine | 2 | g |
| Leucine | 10 | g |
| Lysine HCl | 2.98 | g |
| Methionine | 2 | g |
| Phenylalanine | 2 | g |
| Proline | 2 | g |
| Serine | 2 | g |
| Threonine | 2 | g |
| Tryptophan | 2 | g |
| Tyrosine | 2 | g |
| Valine | 2 | g |
| DTT | 0.6 | g |
| NaHCO₃ | 1 | g |
| Lactate | 3.36 | g |
| ATCC Trace Mineral | 10 | ml |
| Mix | | |
| ATCC Vitamin Mix | 10 | ml |
| adjust pH to 7.2 and | | |
| filter sterilized | | |

SRB Base medium used for INSeq library
selection and sulfate cross-feeding experiment

| | | |
|---|---|---|
| CaCl₂•2H₂0 | 0.1 | g |
| KH₂PO₄ | 0.5 | g |
| Resazurin | 0.5 | ml |
| DTT | 0.6 | g |
| NaHCO₃ | 1 | g |
| ATCC Trace Mineral | 10 | ml |
| Mix | | |
| ATCC Vitamin Mix | 10 | ml |
| adjust pH to 7.2 and | | |
| filter sterilized | | |

### EXAMPLES

The following examples illustrate various iterations of the invention. Further details may be in Rey F. E. et al, PNAS 2013, 110: 13582-13587, incorporated herein by reference in its entirety. Sequence data for D. piger GOR1 can be found at gordonlab.wustl.edu/modeling_microbiota/(link: model_gut_microbiota_genomes.tar.gz).

### Example 1

### D. piger is a Common SRB Present in the Fecal Microbiota

Using PCR primers directed against the aprA gene, which encodes the alpha-subunit of the adesnosine-5′-phosphosulfate reductase present in all known SRB, amplicons were generated from fecal samples previously collected from a group of 34 individuals known to harbor SRB (Hansen et al., 2011). Multiplex pyrosequencing of the PCR products [Titanium chemistry; 2406±1696 reads/sample (mean±SD); 361±6 nt/read] revealed that D. piger was the most frequent SRB present [21/34 (60%)]. D. piger was the sole detectable SRB in 12 of the 21 healthy adult subjects (57%) and co-existed with one or two other sulfate reducers, D. intestinalis and an unclassified SRB, in the other individuals (FIG. 1). The observed prevalence of D. piger is consistent with previously published results (Scanlan et al., 2009). The prominence of D. piger, coupled with the fact that we had previously isolated and sequenced a D. piger strain from human feces (D. piger GOR1; Faith et al., 2011), led us to focus on characterizing the niche of this SRB in a gnotobiotic mouse model of the human gut microbiota.

### Example 2

### A Diet with Low Levels of Fermentable Carbohydrates is Associated with Increased Utilization of Host-Derived Glycans and Increased Levels of D. piger

Adult germ-free mice (NMRI inbred strain) were colonized with D. piger GOR1 and eight other sequenced human gut bacterial species. Together, these genomes contain 36,822 predicted open reading frames (ORFs) that encode major metabolic functions present in the distal human gut microbiome of healthy adults (Turnbaugh et al., 2009; Qin et al., 2010; HMP consortium, 2012), including the ability to (i) break down proteins, plant and host-derived polysaccharides (Bacteroides thetaiotaomicron, Bacteroides caccae and Bacteroides ovatus), (ii) consume oligosaccharides and simple sugars (Eubacterium rectale, Marvinbryantia formatexigens, Collinsella aerofaciens, Escherichia coli), and (iii) ferment amino acids (Clostridium symbiosum, E. coli). Table 51 of Rey et al. PNAS 110: 13582-13587 lists the wide range of predicted proteases and carbohydrate active enzymes (CAZymes; i.e., glycoside hydrolases, polysaccharide lyases, carbohydrate esterases) (Rawlings et al., 2012; Cantarel et al., 2009) that are present in this model human gut microbiome, and their distribution among community members.

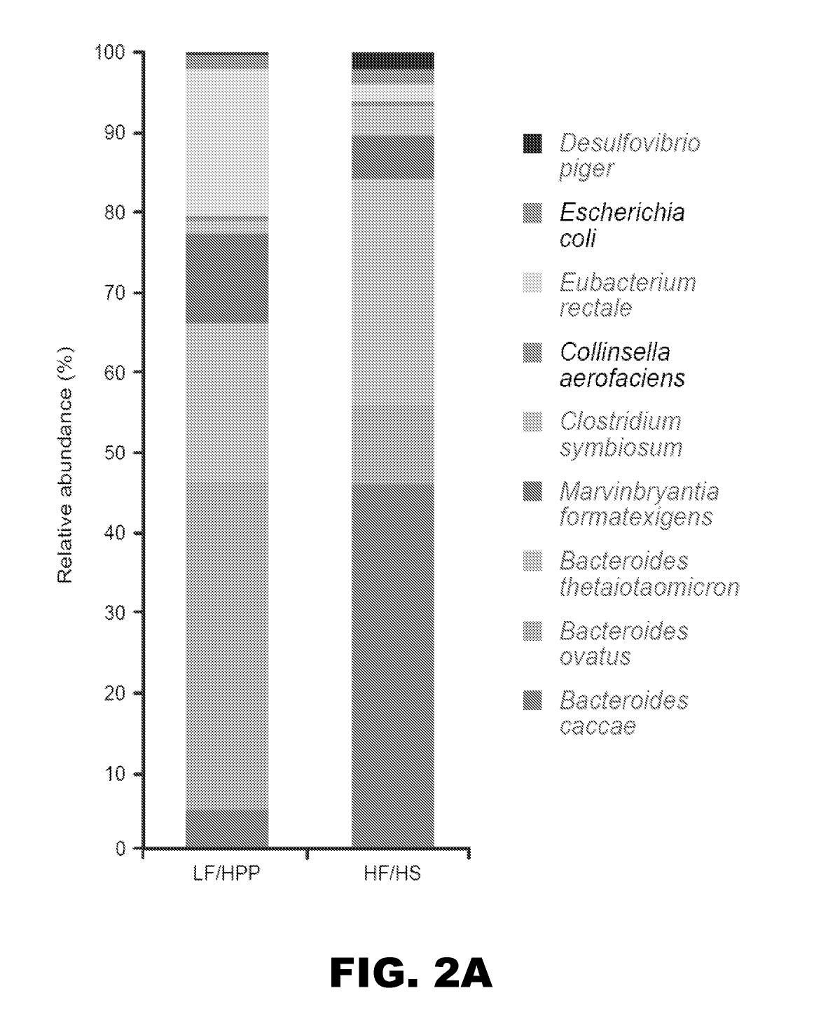

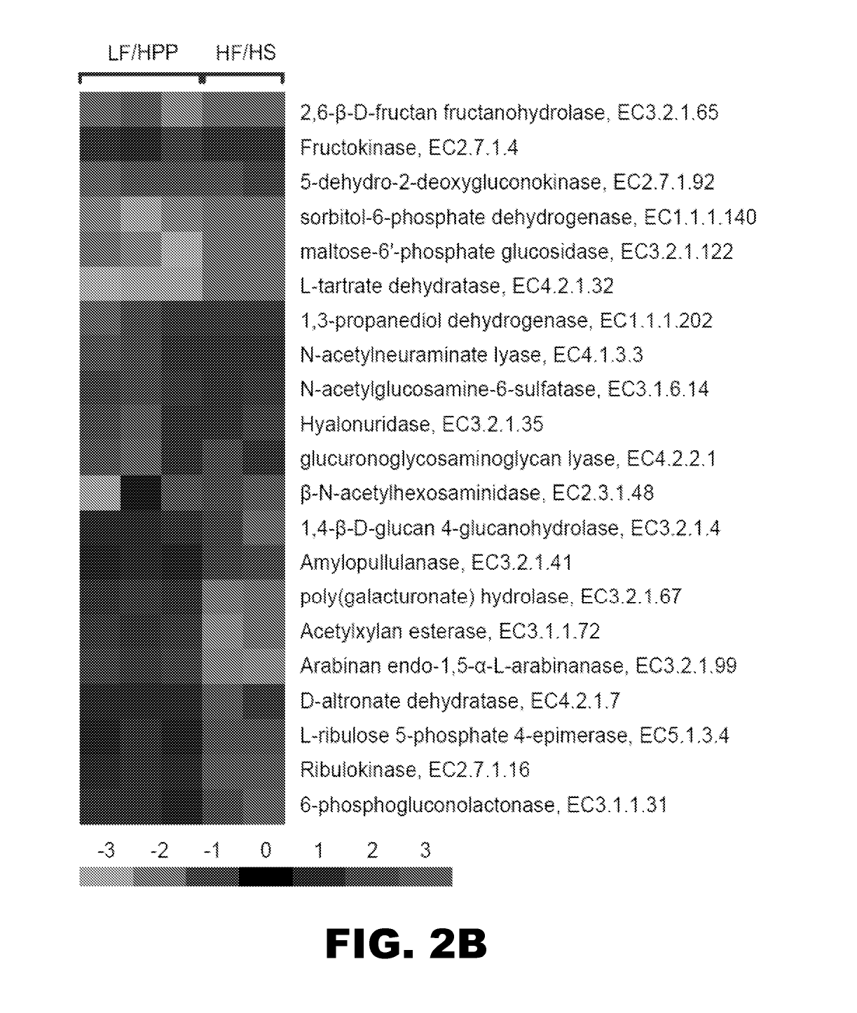

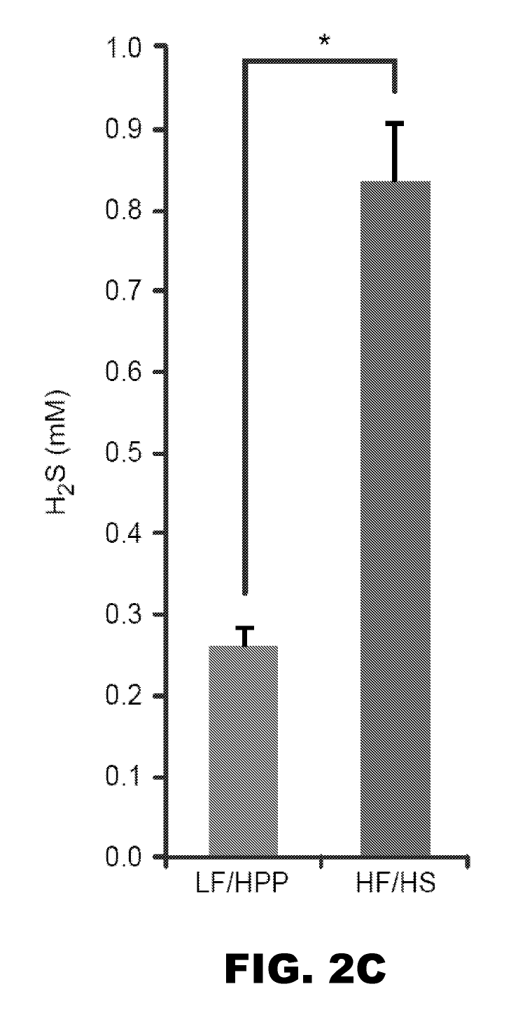

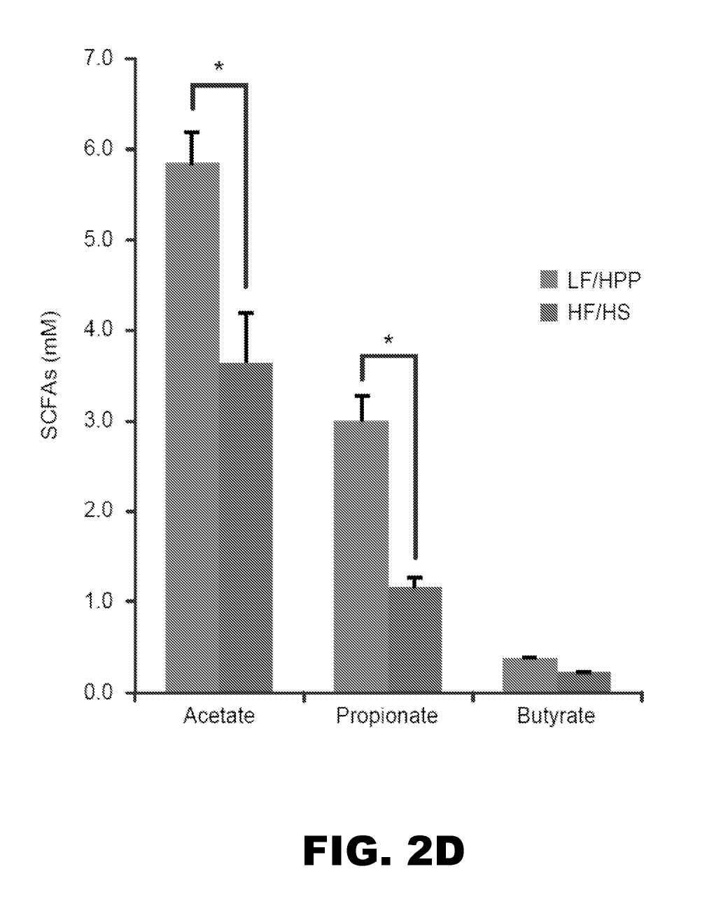

Mice colonized with these nine species were fed one of two different diets ad libitum: one low in fat (4% w/w) and high in plant polysaccharides (abbreviated LF/HPP); the other high in fat (20% w/w) and simple sugars (47% w/w sucrose) (HF/HS; see Table S2 of Rey et al. PNAS 110: 13582-13587 for composition of diets; n=5 mice/diet type). COmmunity PROfiling by shotgun Sequencing (COPRO-Seq) of DNA isolated from fecal samples collected 7 and 14 days after introduction of this nine-member consortium revealed that the relative abundances of five of the nine members were significantly different between mice fed the two different diets (p value <0.05; two-tailed t-test followed by Bonferroni correction). The diet-responsive species included D. piger, which was present at higher levels when mice were consuming the HF/HS diet (FIG. 2A).

To identify microbial functions in D. piger and other members of the community that changed as a function of diet, microbial RNA-Seq analysis of mRNA prepared from fecal samples collected after 14 days on either of the two diets wasp performed (14.0±8.7×10⁶ mRNA reads/sample). mRNA transcripts were functionally grouped based on enzyme commission numbers (ECs) assigned to their protein products (FIG. 2B, Table S3 of Rey et al. PNAS 110: 13582-13587). Among the 1191 ECs detected, 96 were identified that were differentially represented in fecal microbiomes as a function of diet (threshold cutoffs; fold-difference >2, PPDE>0.95; Cyber-T; Table S3 of Rey et al. PNAS 110: 13582-13587). Many of these enzymes participate in various facets of carbohydrate metabolism. For example, the microbiota of mice fed the LF/HPP diet exhibited significantly higher expression of genes encoding ECs involved in (i) the breakdown of plant-derived polysaccharides present in this diet, including xylans (EC3.1.1.72, acetylxylan esterase), β-glucans (EC3.2.1.4, β-glucan hydrolase), pectins (EC3.2.1.67, polygalacturonate hydrolase) and arabinans (EC3.2.1.99, endo-arabinanase, EC3.2.1.55 arabinofuranosidase), and (ii) metabolism of the resulting monosaccharides [arabinose present in arabinans and pectins (EC2.7.1.16, ribulokinase and EC5.1.3.4, L-ribulose 5-phosphate 4-epimerase); and galacturonic acid present in pectins (EC4.2.1.7, D-altronate dehydratase)] (FIG. 2B, Table S3 of Rey et al. PNAS 110: 13582-13587). In contrast, the microbiota of mice fed the HF/HS diet exhibited higher levels of expression of genes involved in (i) the metabolism of sucrose (EC2.7.1.4, fructokinase), sorbitol (EC1.1.1.140, sorbitol dehydrogenase), glycerol (e.g., EC1.1.1.202, 1,3-propanediol dehydrogenase) and myo-inositol (EC1.1.1.18, myo-inositol dehydrogenase), (ii) the breakdown of host-derived mucus glycans (e.g., EC4.1.3.3, N-acetylneuraminate lyase; EC3.2.1.35, hyaluronidase), and (iii) the removal of sulfate from sulfated glycans (EC3.1.6.14, N-acetylglucosamine-6-sulfatase) (FIG. 2B, Table S3 of Rey et al. PNAS 110: 13582-13587).

The contributions of individual species to the pool of ECs differentially represented in the fecal metatranscriptomes of mice consuming the LF/HPP versus HF/HS diets are presented in Table S3 of Rey et al. PNAS 110: 13582-13587. Transcriptional changes in genes encoding enzymes predicted to be involved in the breakdown of dietary and host polysaccharides were largely driven by Bacteroides species; B. ovatus, and to a lesser extent B. thetaiotaomicron, made the biggest contribution to ECs involved in the breakdown of plant polysaccharides that were overrepresented in LF/HPP diet (e.g., EC3.2.1.4, β-glucan hydrolase, EC3.2.1.99, endo-arabinanase) while transcripts from B. caccae and B. thetaiotaomicron drove the observed increase in the abundance of ECs predicted to breakdown host polysaccharides including sulfated mucins (e.g., EC4.1.3.3, N-acetylneuraminate lyase; EC3.2.1.35, hyaluronidase; EC3.1.6.14, N-acetylglucosamine-6-sulfatase).

Chemostat experiments have suggested that liberation of sulfate from sulfated mucins promotes growth of SRB (Willis et al., 1996; Gibson et al., 1988). Consistent with these observations, it was found that the increased sulfatase (EC3.1.6.14) gene expression in Bacteroides species in mice harboring the 9-member community and consuming the HF/HS diet was associated with higher relative levels of D. piger and higher cecal levels of H₂S compared to mice on the LF/HPP diet (FIG. 2A-C and Table S3 of Rey et al. PNAS 110: 13582-13587). Additionally, targeted GC-MS analysis of cecal contents revealed higher levels of bacterial fermentation products (acetate, propionate, and butyrate) in mice fed the LF/HPP versus HF/HS diet (FIG. 2D; p<0.05 two-tailed t-test).

These results suggest that D. piger benefits from diets that provide low levels of fermentable carbohydrates to the distal gut. This benefit may reflect the fact that the polysaccharide-poor HF/HS diet results in increased utilization of host sulfated glycans by members of the model human microbiota, thereby providing free sulfate to D. piger.

### Example 3

### Transposon Mutagenesis Identifies Key Determinants for D. piger Fitness In Vivo

A genome-wide transposon mutagenesis method known as INsertion Sequencing (INSeq) (Goodman et al., 2009) was used to define D. piger fitness determinants in various nutrient contexts. INSeq uses a modified mariner transposon that contains Mmel restriction enzyme sites at its ends, allowing capture of 16-17 bp of flanking chromosomal DNA adjacent to the site of transposon insertion. A population of transposon mutants is generated from a sequenced bacterial species, with each mutant strain containing a single site of transposon insertion. The resulting library of tens of thousands of mutants is then subjected to an in vitro or in vivo selection. DNA sequencing of the transposon and flanking chromosomal DNA liberated by Mmel permits the location and abundance of each transposon mutant in the library. The number of sequencing reads for each mutant in the ‘output’ population that was subjected to a given selection is compared to the sequencing reads obtained from the ‘input’ population. This ratio (number of reads in the output/number of reads in the input) provides information about the effect each transposon insertion has on the fitness of the organism under the selection condition applied. Transposon insertions in genes that result in reduced fitness under a given selective pressure will have a reduced abundance of reads relative to those observed in the input library.

An isogenic library composed of 30,000 unique transposon mutants of D. piger was constructed (inter- and intragenic insertions). The library was generated under strict anaerobic conditions using a rich medium, allowing us to obtain mutants in genes involved in a wide range of metabolic functions. INSeq analysis revealed that the library was composed of transposon insertions in 2,181 of the 2,487 predicted ORFs in the D. piger GOR1 genome. Of the 306 ORFs without observed transposon insertions, we predict that 174 ORFs likely encode genes that are essential for the growth of D. piger on rich medium; they include genes involved in ‘core functions’ such as cell division, protein translation, and cell wall biosynthesis (Table 1).

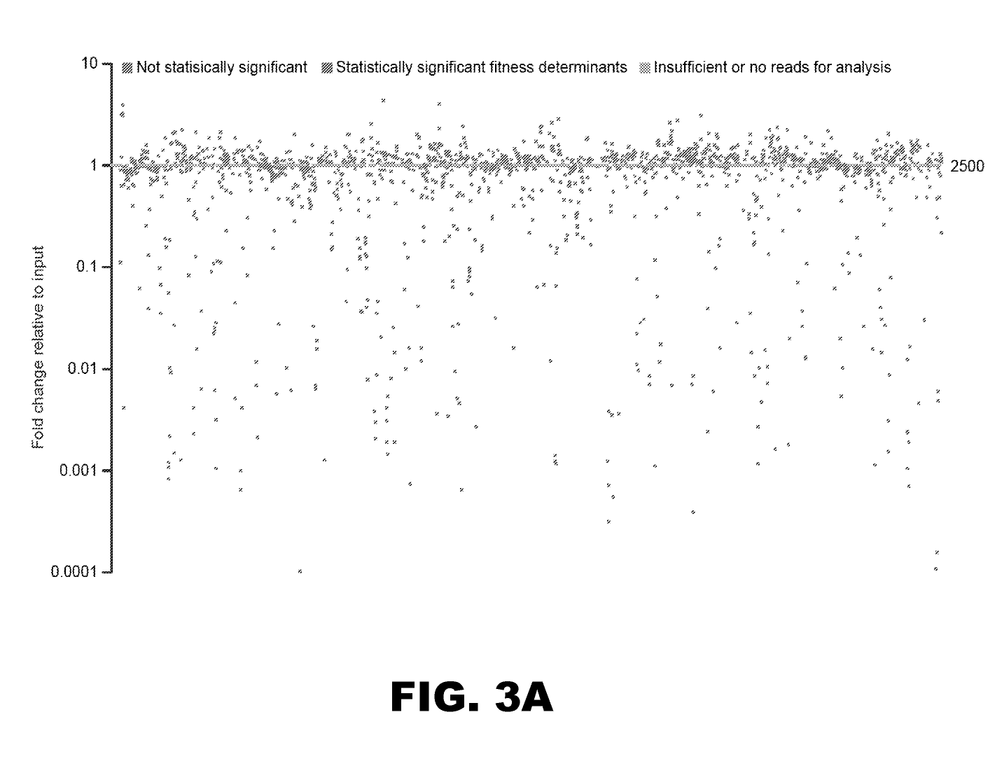

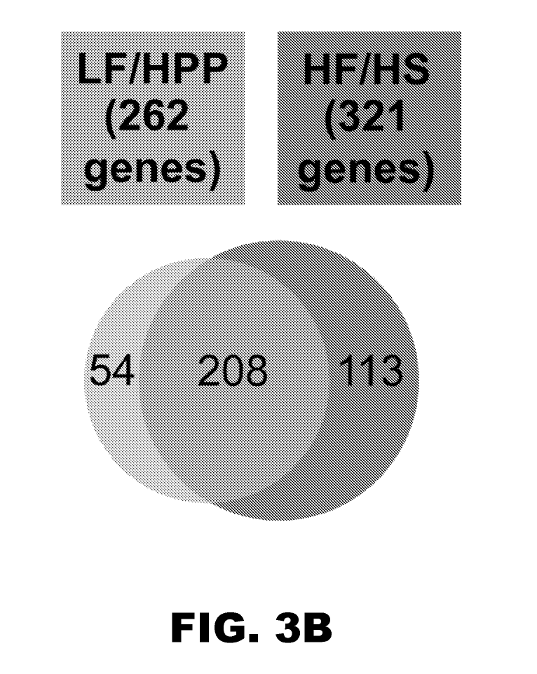

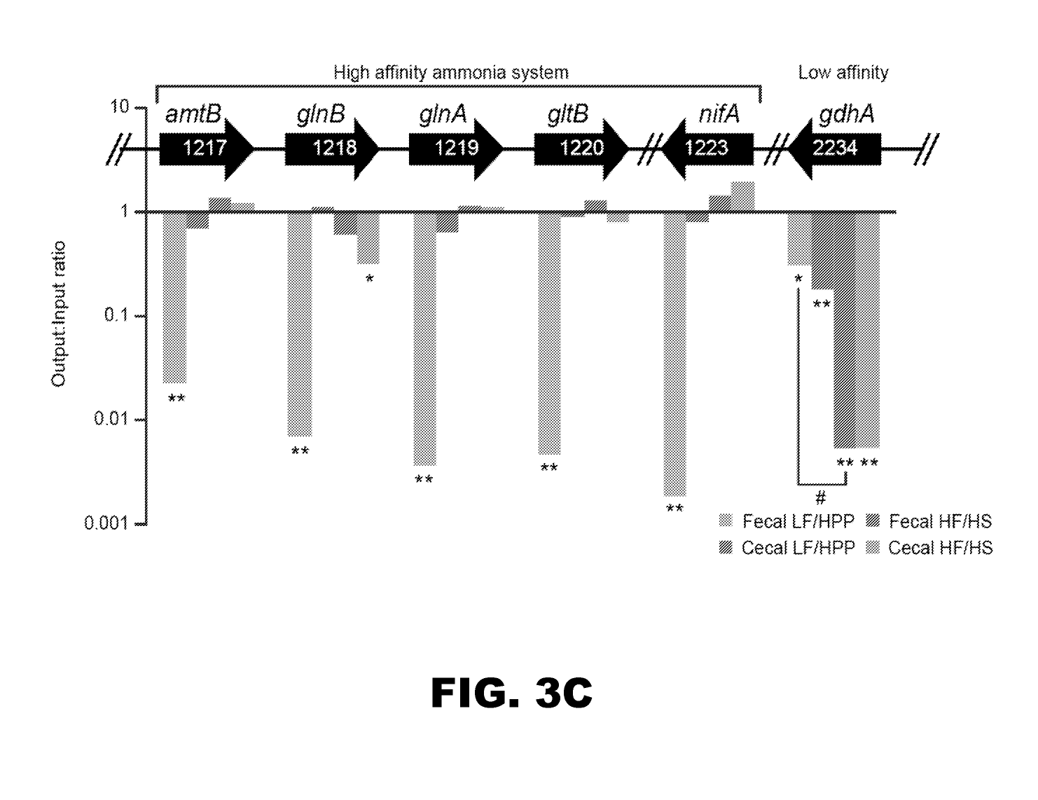

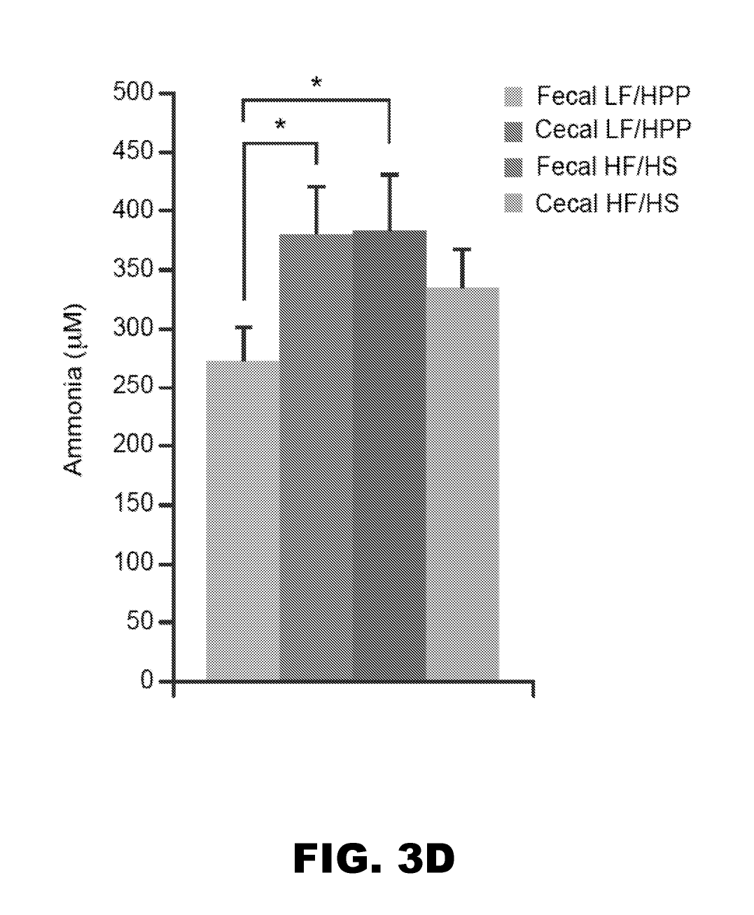

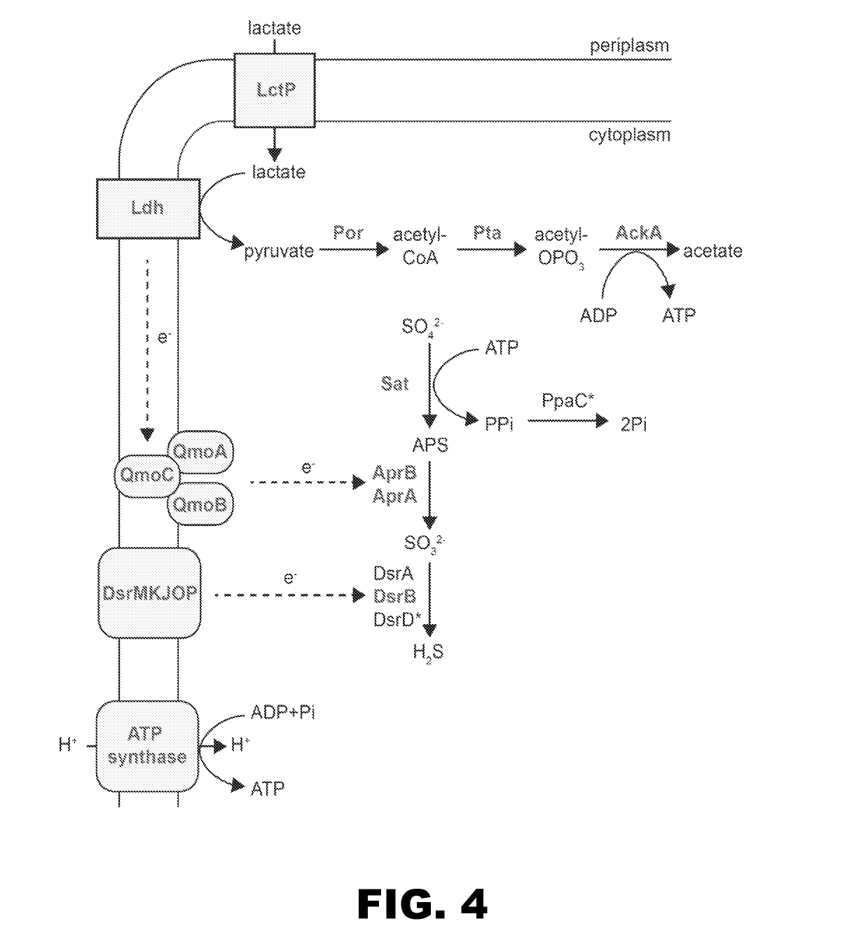

The mutant library was first characterized in vitro, applying a growth selection in a fully defined medium containing all 20 amino acids, lactate (source of carbon and reducing equivalents) and sulfate (electron acceptor). 266 genes were identified that when disrupted by a transposon had significantly reduced fitness under these conditions (p_{adj}<0.05, output:input ratio <0.3; FIG. 3A). They included genes involved in pyrimidine and purine biosynthesis, lactate utilization, gluconeogenesis and sulfate-reduction (Table S5 of Rey et al. PNAS 110: 13582-13587; FIG. 4 presents a pathway map for sulfate reduction showing fitness determinants disclosed by the transposon mutagenesis screen). With the exception of arginine, genes involved in amino acid biosynthesis were generally not required for growth in this amino acid-rich medium (Table S5 of Rey et al. PNAS 110: 13582-13587).

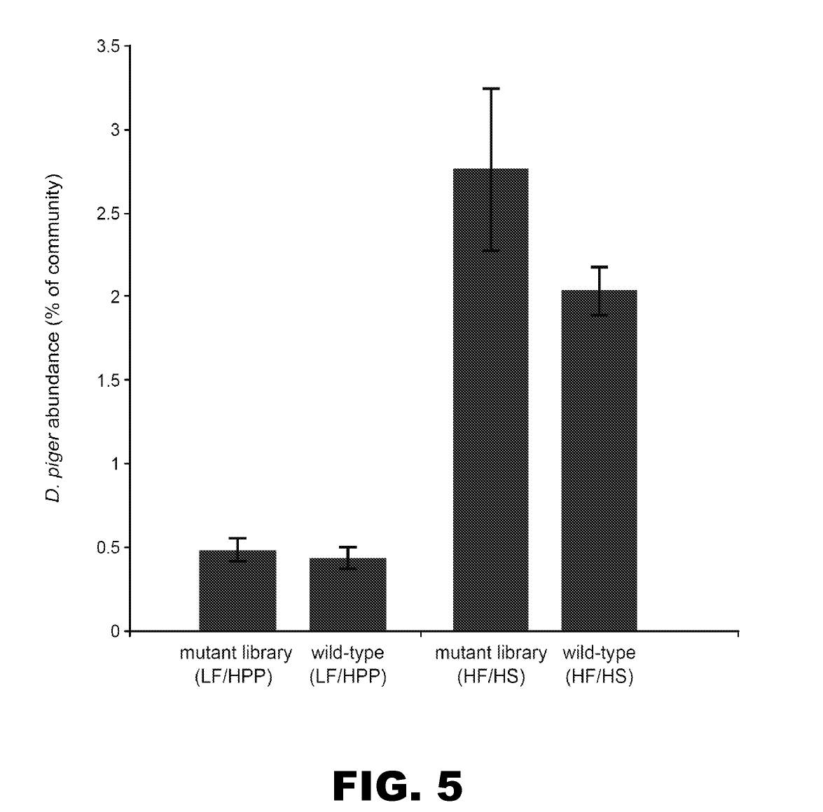

Next, the D. piger mutant library was introduced by gavage into gnotobiotic mice colonized with the same eight species mentioned above. Mice colonized with the eight-member community were fed either the LF/HPP or HF/HS diet for 14 days before introduction of the D. piger mutant library and remained on these diets for the duration of the experiment. COPRO-Seq analysis of fecal pellets obtained 7 days after inoculation of the mutant library indicated that the relative abundance reached by the aggregate pool of transposon-mutants was not significantly different than the abundance achieved by wild-type D. piger in mice on the same diets (FIG. 5). The ability of D. piger to colonize an established community to levels similar to those reached when gavaged with the 8-member community (FIG. 5) highlights its capacity to invade. INSeq analysis of fecal pellets obtained at the time of sacrifice 7 days after gavage revealed mutations in 262 and 321 genes that produced a significant reduction in invasiveness/fitness (FDR p_{adj}<0.05, output:input ratio <0.3) in mice consuming LF/HPP and HF/HS diets, respectively. Two hundred and eight of these fitness determinants are shared between both diet selections (FIG. 3B, Table S6 of Rey et al. PNAS 110: 13582-13587) and their fitness effects were comparable in the cecal and fecal microbiota (more than 78% of fitness determinants were shared between fecal and cecum in each diet context), including many genes known or predicted to be involved in amino acid metabolism, carbohydrate metabolism, energy metabolism, membrane transport, and nucleotide metabolism (Table S7 of Rey et al. PNAS 110: 13582-13587). These likely represent core fitness determinants for establishment and maintenance of D. piger in the gut, at least in the context of the two diets tested.

The fitness effects of 167 genes were differentially affected by diet (Table 2). For example, the LF/HPP and HF/HS diets select for genes involved in distinct ammonia assimilation pathways (FIG. 3C). Ammonia can serve as a source of nitrogen that is incorporated into glutamate and glutamine and then transferred to other nitrogen-containing components (e.g., other amino acids, purines, pyrimidines, amino sugars). Incorporation of ammonia can occur in an energy-dependent or -independent manner depending upon whether the concentration of ammonia is low or high, respectively. We found that genes predicted to be involved in ammonia assimilation under limiting conditions (high affinity ammonia system), including an ammonia transporter [DpigGOR1_{—}1217 (amtB)], two nitrogen regulatory proteins [DpigGOR1_{—}1218 (glnB), DpigGOR1_{—}1223 (nifA)], glutamine synthase [DpigGOR1_{—}1219 (glnA)] and glutamate synthase [DpigGOR1_{—}1220 (gltB)], are important for fitness when mice are fed the LF/HPP but not the HF/HS diet (FIG. 3C). In contrast, transposon disruption of the gene encoding glutamate dehydrogenase [DpigGOR1_{—}2234 (gdhA)], an enzyme involved in ammonia assimilation when levels are high (low affinity ammonia system), resulted in a strong fitness defect in mice fed the HF/HS diet, but had a significantly smaller effect in mice consuming the LF/HPP diet (FIG. 3C). Consistent with these findings, we detected significantly lower levels of ammonia in fecal pellets collected from mice fed the LF/HPP diet compared to their HF/HS diet-consuming counterparts (FIG. 3D).

Although transposon disruption of genes involved in the high affinity ammonia assimilation pathway resulted in lower D. piger abundance in the fecal microbiota of LF/HPP-fed mice, we observed no fitness defect in the cecal microbiota (FIG. 3C). In contrast, disruption of the gene encoding glutamate dehydrogenase [DpigGOR1_{—}2234 (gdhA)] from the low affinity system had a significantly larger effect (lower abundance of mutants in this gene) in the cecal compared to fecal microbiota of LF/HPP-fed mice (see FIG. 3C which also shows that the differential fitness effects of gdhA disruption in the cecal compared to fecal microbiota are diet-dependent; they are not observed on the HF/HS diet). The differential effects of diet and location on the fitness contributions of genes involved in distinct ammonia assimilation pathways can be explained by the significantly lower ammonia levels in feces compared to cecal contents of mice fed the LF/HPP diet; this difference is not observed in the HF/HS diet (FIG. 3D).

Genes involved in H₂ consumption and sulfate reduction are required for optimal in vivo colonization of D. piger in both diet contexts; they include (i) a predicted periplasmic [NiFeSe] hydrogenase complex (DpigGOR1_{—}1496-DpigGOR1_{—}1497) important in other Desulfovibrio species for growth in H₂ (Caffrey et al., 2007), (ii) hydrogenase maturation genes (DpigGOR1_{—}0739-DpigGOR1_{—}0740), (iii) a predicted transport system for nickel, which functions as an important cofactor for the hydrogenase (DpigGOR1_{—}1393-DpigGOR1_{—}1398), (iv) a high molecular weight cytochrome complex, Hmc (DpigGOR1_{—}0741-DpigGOR1_{—}0744) and the QmoABC complex (DpigGOR1_{—}0790-DpigGOR1_{—}0792) which are two electron transport systems required for sulfate reduction in other species (Dolla et al., 2000; Keon et al., 1997; Zane et al., 2010), plus (v) components of sulfite reductase (DpigGOR1_{—}0170-DpigGOR1_{—}0174). These results emphasize the importance of hydrogen metabolism and sulfate respiration and/or other oxidized sulfur compounds for survival of D. piger in the distal gut and underscore the restricted metabolic options that D. piger has to efficiently generate energy in this environment.

### Example 4

### Comparison of In Vitro and In Vivo D. piger Fitness Determinants

We subjected the D. piger mutant library to another set of selections in vitro, this time using various electron donors for sulfate reduction (formate, H₂, lactate or pyruvate). We also tested fermentative growth (i.e. the ability to grow without sulfate using pyruvate as the sole carbon and energy source). INSeq revealed a set of genes involved in numerous functions important for growth (e.g., sulfate reduction, purine and pyrimidine biosynthesis, and ATP synthesis) that were also critical for fitness in vivo (Table S9 of Rey et al. PNAS 110: 13582-13587). Transposon insertions in the periplasmic [NiFeSe] hydrogenase genes (DpigGOR1_{—}1496-DpigGOR1_{—}1497) important for gut colonization (see above), resulted in in vitro growth defects in the presence of H₂ but not with the other electron donors. In contrast, genes required for optimal growth and survival in vitro with formate [e.g., formate dehydrogenase encoded by DpigGOR1_{—}0133-DpigGOR1_{—}0135], or lactate [e.g., the lactate transporter specified by DpigGOR1_{—}1075; and lactate dehydrogenase (DpigGOR1_{—}0371)] were not required for fitness in vivo. The finding that genes required for optimal growth in vivo do not overlap with those specifically required for optimal growth in vitro with formate, lactate, and pyruvate suggests that D. piger either does not use these electron donors in vivo, or uses several different electron donors, and/or that disruption of one pathway is compensated by another pathway.

The list of in vivo-specific fitness determinants included members of a locus that encodes rubredoxin:oxygen oxidoreductase (DpigGOR1_{—}1319), rubredoxin (DpigGOR1_{—}1321) and rubredoxin oxidoreductase (DpigGOR1_{—}1322), and a locus encoding subunits of a cytochrome bd oxidase (DpigGOR1_{—}1865-DpigGOR1_{—}1866). These genes are known to be important for handling oxygen and oxidative stress (Gomes et al., 1997; Auchere et al., 2006; Wildschut et al., 2006; Voordouw and Voordouw, 1998; Lumppio et al., 2001). D. piger could experience varying degrees of oxidative stress during the process of gavage into gnotobiotic animals, during transit from the proximal to the distal gut and/or as it associates with the gastrointestinal mucosa (a microhabitat that is exposed to higher oxygen levels due the extensive submucosal capillary network that underlies it compared to the intestinal lumen; Zinkevich and Beech, 2000; Fite et al., 2004; Nava et al., 2012).

Table 3 groups genes that have significant fitness effects in vivo but not in vitro into those that exhibit diet-independence or diet-dependence.

### Example 5

### B. thetaiotaomicron Boosts D. piger Growth In Vitro and In Vivo Through Provision of Free Sulfate

Potential in vivo sources of sulfate for D. piger include the host diet, sulfated oligosaccharide side chains of glycosaminoglycans in host mucins, and sulfonic acid moieties in bile acids. Accessing these host sources of sulfate requires their liberation by sulfatases, an enzymatic activity encoded by members of the microbiota (Salyers and O'Brien, 1980). Bacterial sulfatases require a sulfatase maturation enzyme for a post-translational modification (oxidation) of their active-site cysteine or serine to C_{α}-formylglycine (Benjdia et al., 2011). One D. piger gene (DpigGOR1_{—}2296) encoding a protein with a predicted Pfam sulfatase domain was identified, but the Blastp E-value was low compared to other known sulfatases (e.g., 3.4×10⁻⁷ versus 6×10⁻⁶⁰ for the sulfatase encoded by B. thetaiotaomicron locus BT3051). In addition, a D. piger gene encoding a sulfatase-maturation enzyme was not identified. Therefore, it was hypothesized that D. piger lacks an endogenous mechanism to liberate host sulfate and may benefit from other bacterial species capable of liberating sulfate from a diverse array of sulfated host glycans. One member of the model community used in this study, Bacteroides thetaiotaomicron, has demonstrated sulfatase activity that is required for its adaptive foraging of mucosal glycans when the host diet lacks complex polysaccharide substrates (Benjdia et al., 2011). Despite the presence of 28 putative sulfatase genes, B. thetaiotaomicron encodes only one sulfatase maturation enzyme (BT0238) that is essential for its sulfatase activity (Benjdia et al., 2011).

Since it was unclear if sulfate liberated by B. thetaiotaomicron from host mucosal glycans would be available to D. piger, experiments were initially performed to determine the potential for cross-feeding between these two bacteria in a simplified and defined in vitro system. A B. thetaiotaomicron strain Δbt0238 that lacks detectable sulfatase activity, and the isogenic wild-type strain were grown in separate cultures containing minimal medium with either a sulfated or non-sulfated carbon substrate (chondroitin sulfate and fructose, respectively). The resulting conditioned medium, after filter sterilization, was used as a potential source of sulfate for D. piger. The conditioned medium was supplemented with lactate as the sole carbon and electron source for D. piger (lactate does not support growth of D. piger in the absence of sulfate; data not shown).

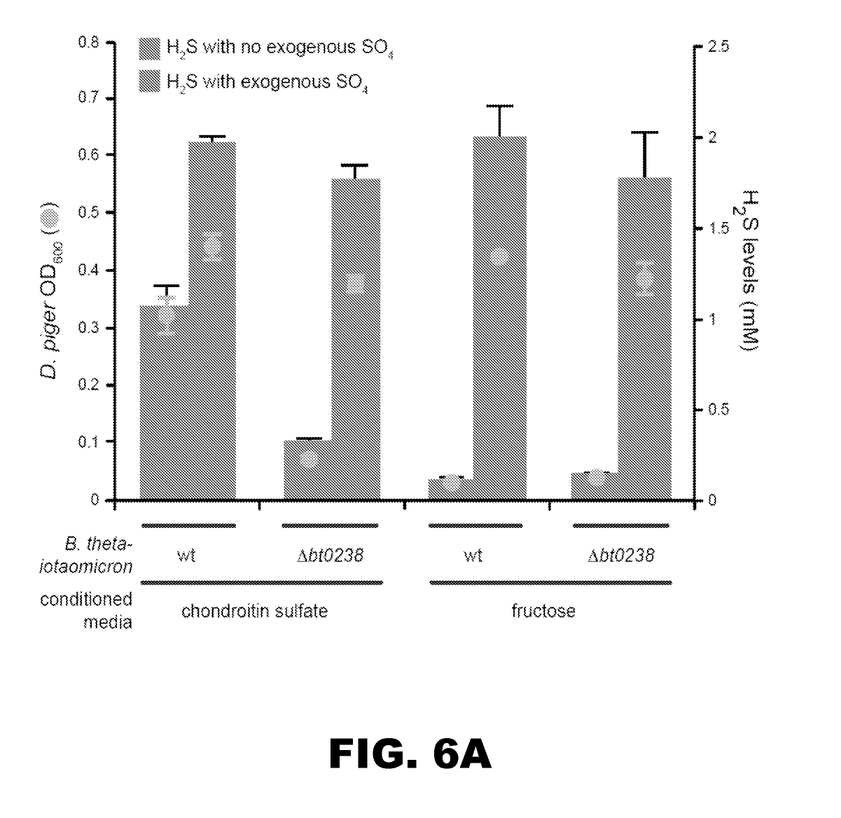

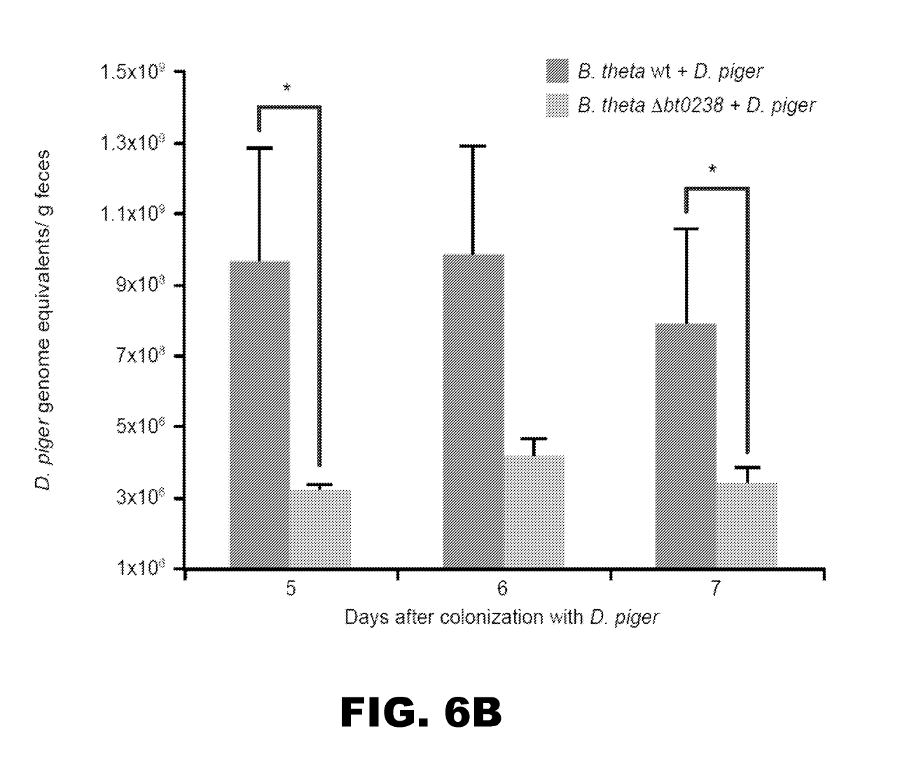

Wild-type B. thetaiotaomicron grew in minimal medium containing chondroitin sulfate, whereas the Δbt0238 strain, which lacks the sulfatase maturation enzyme and hence is deficient in sulfatase activity, failed to grow. In contrast, both the wild-type and mutant B. thetaiotaomicron strains grew in minimal medium containing fructose as the carbon source. Growth of D. piger was only observed in conditioned medium obtained from wild-type B. thetaiotaomicron cultured in the presence of chondroitin sulfate (FIG. 6A). The lack of growth of D. piger in the fructose-conditioned medium was not due to inhibitory effects, since addition of exogenous sulfate allowed growth (FIG. 6A). The inability of D. piger to grow in the chondroitin sulfate-containing medium harvested from cultures of B. thetaiotaomicron Δbt0238 shows that D. piger is not able to metabolize chondroitin sulfate. H₂S measurements confirmed that the growth observed with conditioned chondroitin sulfate-containing medium correlates with sulfate reduction (FIG. 6A). Together, these in vitro results indicate that B. thetaiotaomicron can liberate sulfate from glycans that then becomes available for D. piger, and that this cross-feeding activity ultimately depends on the sulfatase maturation enzyme of B. thetaiotaomicron.

To examine the role of sulfate cross-feeding between B. thetaiotaomicron and D. piger in gnotobiotic mice, adult germ-free animals were mono-colonized with a single oral gavage of wild-type or Δbt0238 B. thetaiotaomicron strains. Mice were fed the HF/HS diet for one week prior to a second gavage with wild-type D. piger. This diet was chosen because it results in increased expression of B. thetaiotaomicron sulfatase genes as well as genes involved in utilization of host glycans (FIG. 2B), thereby permitting adaptive foraging of sulfated host glycans. qPCR analysis of fecal pellets collected 5, 6 and 7 days after introduction of D. piger revealed that its abundance in mice co-colonized with B. thetaiotaomicron Δbt0238 was significantly lower than in mice co-colonized with the isogenic wild-type B. thetaiotaomicron strain (FIG. 6B). These results indicate that sulfate cross-feeding by bacteria with sulfatase activity supports higher levels of intestinal colonization by D. piger. However, because D. piger was still able to colonize mice associated with the mutant B. thetaiotaomicron strain there appear to be other available sources of oxidized sulfur, including the diet. These sources were searched for in follow-up experiments involving a series of diets containing different sources and levels of sulfur.

### Example 6

### Supplementation of Diet with a Sulfated Glycosaminoglycan (Chondroitin Sulfate) Increases Levels of D. piger Colonization

Sulfate and sulfite are commonly used as preservatives and antioxidants in a variety of foods (bread, preserved meat, dried fruit, wine). Sulfate is also present in the commonly used dietary supplement chondroitin sulfate and in food additives (carrageenan). To test how different dietary sulfur sources affect D. piger colonization levels, 12 diets were generated, all based on the HF/HS diet that contains 0.12% (w/w) sulfate. In these diets the sulfate concentration was deliberately modified over a 600-fold range (from 0.001% to 0.6% w/w), and introduced sulfur compounds with different redox states (e.g., sulfate versus thiosulfate versus sulfite). Since the gut has a large absorptive capacity for sulfate and likely related compounds (Curno et al., 2008) sulfate availability was also manipulated by constructing a diet with a glycan-bound source of sulfate (chondroitin sulfate) that is poorly absorbed in the small intestine (Barthe et al., 2004) (see Table 16 for the composition of all diets). Six groups of gnotobiotic mice, each composed of two co-housed animals colonized with the nine-member community were fed one of the 13 diets (the unmodified HF/HS diet served as a reference control). A sequence of five different diets was administered to each set of mice. Each diet was given for 1 week. All mice began with the baseline HF/HS diet. The order of presentation of the four subsequent diets, and diet type were randomized among the six groups so that in the end each diet had been administered to two different sets of mice (n=4 animals; Table S11 of Rey et al. PNAS 110: 13582-13587).

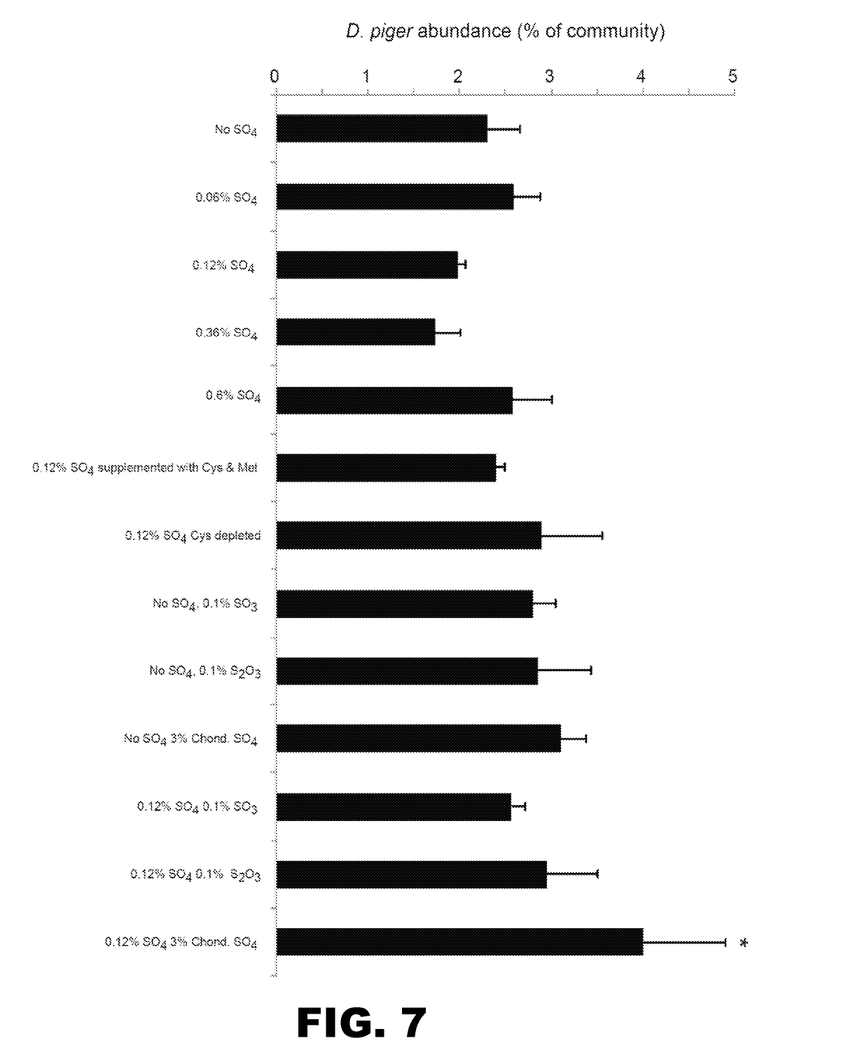

A 600-fold change in dietary sulfate levels did not affect the relative abundance of D. piger in the 9-member model human microbiota (FIG. 7). The lack of a reduction in D. piger levels with administration of the lowest sulfate diet (0.001% w/w) suggested that D. piger either predominately uses host-derived sulfate or that under these dietary conditions D. piger uses an alternative pathway for energy generation instead of sulfate reduction. To differentiate between these possibilities, mice were colonized with the 8-member community and fed the low sulfate diet (0.001% w/w) or the control HF/HS diet prior to and for 7 days after gavage with the D. piger mutant library. INSeq analysis of fecal samples obtained 7 days after introduction of the mutant library revealed 291 genes as important fitness determinants for both the low and standard sulfate diets (out of a total of 384 unique fitness determinants; see Table S12 of Rey et al. PNAS 110: 13582-13587 for a list of shared as well as diet-specific fitness factors). Importantly, we found that all of the sulfate reduction and hydrogenase genes are important for fitness in the low sulfate diet context, just as they were with the standard HF/HS diet.

Together, these results indicate that although the ability to reduce sulfate is critical for D. piger colonization of the intestine, dietary free sulfate is not a necessary contributor to D. piger colonization levels and that, and at least in our model human gut community, D. piger can use sulfate from sources other than diet (e.g., the host) without a decrease in its representation. Supplementation of the HF/HS diet with 3% chondroitin sulfate doubled D. piger levels relative to the HF/HS diet (FIG. 7; p<0.05; one-way ANOVA and Dunnett's post-hoc test). These latter findings provided a means to test the effect of manipulating levels of D. piger on other members of the community and on host physiology.

### Example 7

### High Levels of D. piger Produced by Chondroitin Supplementation Decreases Oxidative Metabolism in the Mouse Gut

To assess the impact that diet-induced increases in the levels of D. piger has on the microbiota and the host, seven week-old germ-free mice were colonized with either the 8-member community that lacks this SRB or with the D. piger-containing nine-member bacterial consortium. Animals were fed the HF/HS diet supplemented with 3% chondroitin sulfate for two weeks (n=4-5 mice/community). COPRO-Seq was used to determine the relative abundance of each member of the community, (ii) RNA-Seq to profile the microbial community and proximal colonic responses to D. piger, and (iii) gas chromatography and ultra high performance liquid chromatography mass spectrometry (UPLC-MS) to assess metabolic changes that result from co-colonization with D. piger.

The presence of D. piger was associated with a significant increase in the representation of C. aerofaciens and a decrease in E. coli (FIG. 10A). Furthermore, Spearman correlation analysis of the relative abundance of D. piger and C. aerofaciens in the fecal microbiota of mice containing the nine-member community who were fed all of the diets described above (LF/HPP plus the 13 HF/HS-based diets) revealed a significant positive association between the levels of these two species [r=0.376, P=0.001 (r=0.562, P=0.003 if only the 2-wk diet exposures with LF/HPP, HF/HS, and HS/HS+3% chondroitin sulfate are considered)]. The main products of C. aerofaciens fermentation are lactate, H₂, and formate, all of which serve as substrates for D. piger growth (Loubinoux et al. 2002), GC-MS disclosed that lactate levels were lower in the cecal contents of mice harboring D. piger (FIG. 10B) Higher levels of D. piger may contribute to increased levels of C. aerofaciens promoting more efficient fermentation through removal of H₂ and formate.

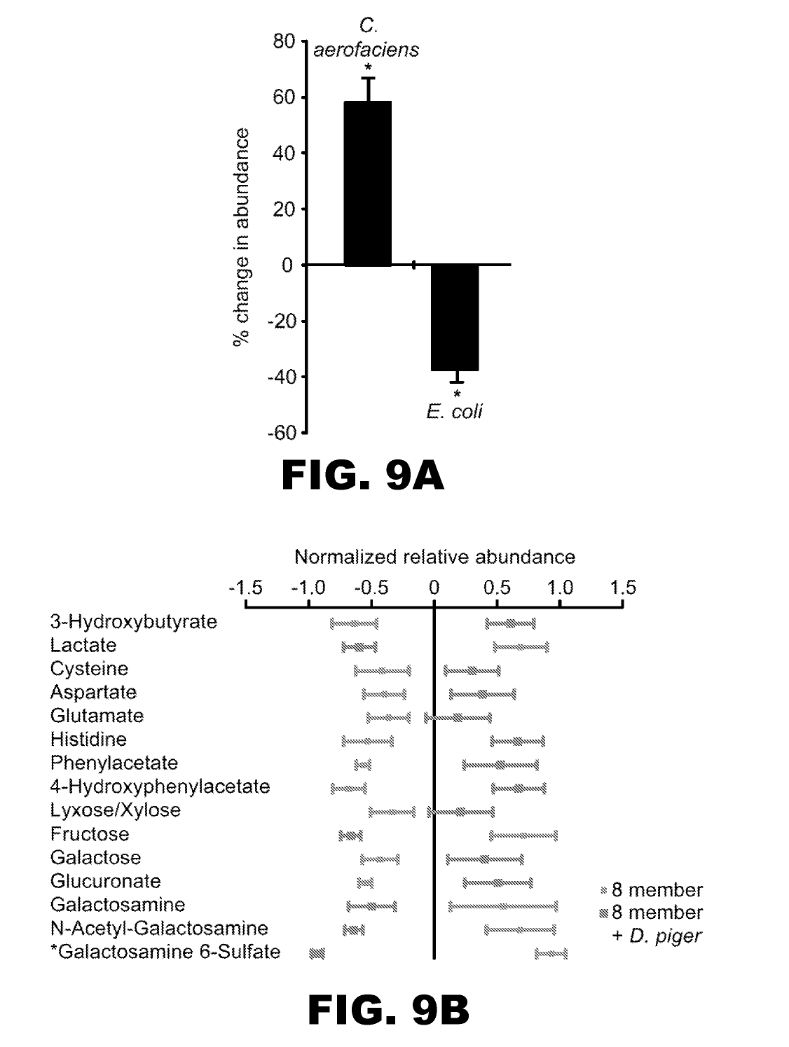

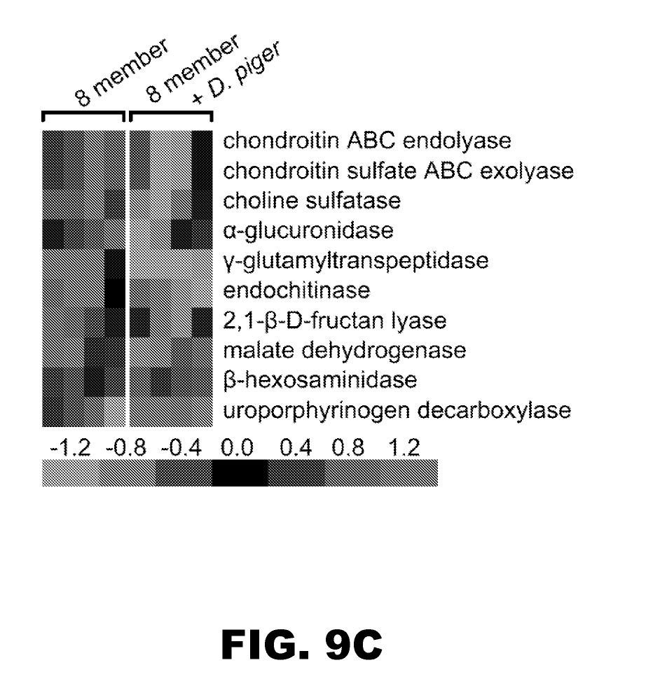

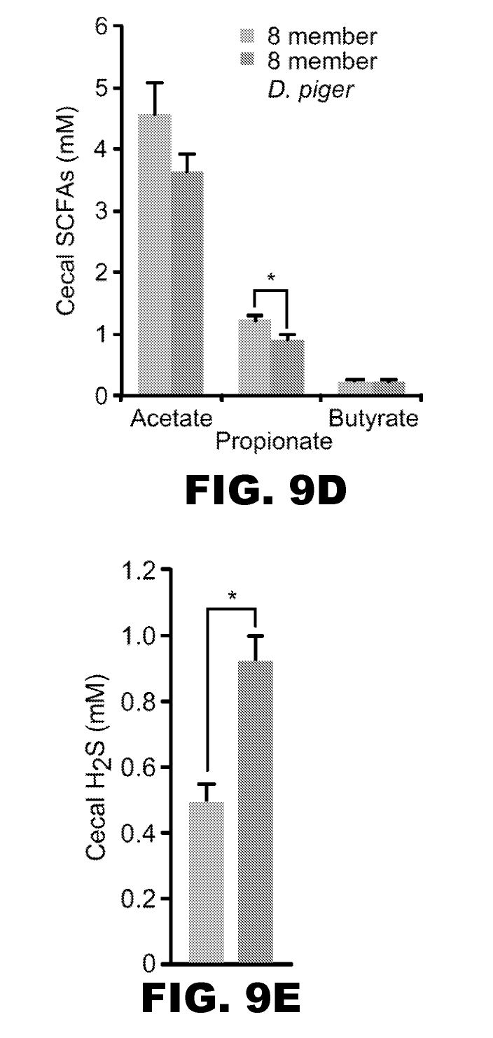

Microbial RNA-Seq analysis of the fecal metatranscriptome revealed that genes encoding malate dehydrogenase (EC1.1.37; 9C) exhibited lower levels of expression in the presence of D. piger. This change was largely driven by changes in expression in B. caccae, B. ovatus and B. thetaiotaomicron. Malate dehydrogenase is involved in the NADH-consuming step that converts oxaloacetate into malate, which in turn is used for the production of succinate or propionate in Bacteroides sp. Consistant with this finding, levels of proprionate, a major end-product of fermentation generated by Bacteroides spp., were lower in the fecal microbiota of mice colonized with D. piger (FIG. 9D).

Untargeted GC-MS and Ultra High-Performance Liquid Chromatography (UPLC)-MS analyses of cecal contents harvested from mice colonized for 2 wk with the eight-member versus nine-member communities indicated that D. piger impacted microbial metabolism of amino acids and carbohydrates. Levels of phenylacetate and 4-hydroxyphenylacetate, two microbial metabolites derived from phenylalanine and tyrosine, respectively, were increased with D. piger colonization. Cecal levels of fructose, N-acetyl galactosamine (one of the alternating sugars of chondroitin sulfate), galactosamine, and galactosamine-6-sulfate were lower with D. piger, whereas glucuronate (the other alternating sugar of chondroitin sulfate) was present at higher levels (FIG. 9B). Glucuronate is more oxidized than N-acetyl galactosamine, and its fermentation results in lower biomass yields per mole of carbohydrate metabolized compared with more reduced carbon sources. Although there were no differences in microbial biomass between the groups of mice (defined by fecal DNA content), microbial RNA-Seq identified several enzymes involved in the degradation of chondroitin sulfate that were expressed at lower levels in the presence of D. piger (i) chondroitin sulfate lyase (EC4.2.2.20; EC4.2.2.21), which degrades chondroitin sulfate into sulfated disaccharides: (ii) a glucuronidase (EC3.2.1.139), which breaks the unsulfated disaccharides from chondroitin sulfate into monosaccharide components, and (iii) N-acetyl-β-hexosaminidase (EC3.2.1.52), which is involved in the degradation of compounds containing terminal N-acetyl hexosamine residues, such as chondroitin sulfate (FIG. 9C, Table 4). These results suggest that in the presence of D. piger, community members require less chondroitin sulfate and prioritize the use of its more reduced carbohydrate moiety (N-acetyl-galactosamine). Utilization of more reduced carbon sources in the presence of D. piger may be facilitated via interspecies formate/hydrogen transfer. Altogether, these findings suggest that in the presence of D. piger, the microbial community (most likely its Bacteroides spp.) ferments substrates more actively: i.e., members of the community consume fewer substrates to maintain the same biomass.

We next assessed the effects of D. piger on host physiology. At high concentrations (mM range), H₂S impairs oxygen consumption by inhibiting cytochrome c oxidase, the terminal oxidase of the mitochondrial respiratory chain. Mice containing D. piger and consuming the HF/HS diet supplemented with chondroitin sulfate had significantly increased cecal levels of H₂S (FIG. 9E) compared with mice consuming the same diet but with the eight-member consortium. Besides short-chain fatty acids, amino acids and ketone bodies (e.g., 3-hydroxybutyrate generated via ketogenesis) serve as respiratory fuels for the gut epithelium. GC-MS of cecal contents disclosed that levels of glutamate, cysteine, aspartate, histidine, and 3-hydroxybutyrate were significantly increased in the presence of D. piger (FIG. 9B). RNA-Seq of mouse gene expression in the proximal colon provided evidence of decreased host consumption of amino acids in HF/HS diet-fed mice colonized with the nine-member compared with the eight-member consortium that lacked this SRB. Oxidation of amino acids results in the production of intracellular ammonia that is subsequently detoxified via the urea cycle. Levels of mRNA encoding carbamoyl-phosphate synthase 1, the enzyme that catalyzes the first committed step of the urea cycle, were 3.8-fold lower (P<0.005; Table 5) in mice harboring D. piger. Moreover, because there were no significant differences in expression of microbial genes involved in the metabolism of these compounds between the two groups of mice, as judged by microbial RNA-Seq, we surmised that the increased cecal levels of amino acids, particularly glutamate, or 3-hydroxybutyrate were not a consequence of reduced microbial consumption or increased production of these metabolites brought about by the presence of D. piger but rather a reflection of reduced host metabolism.

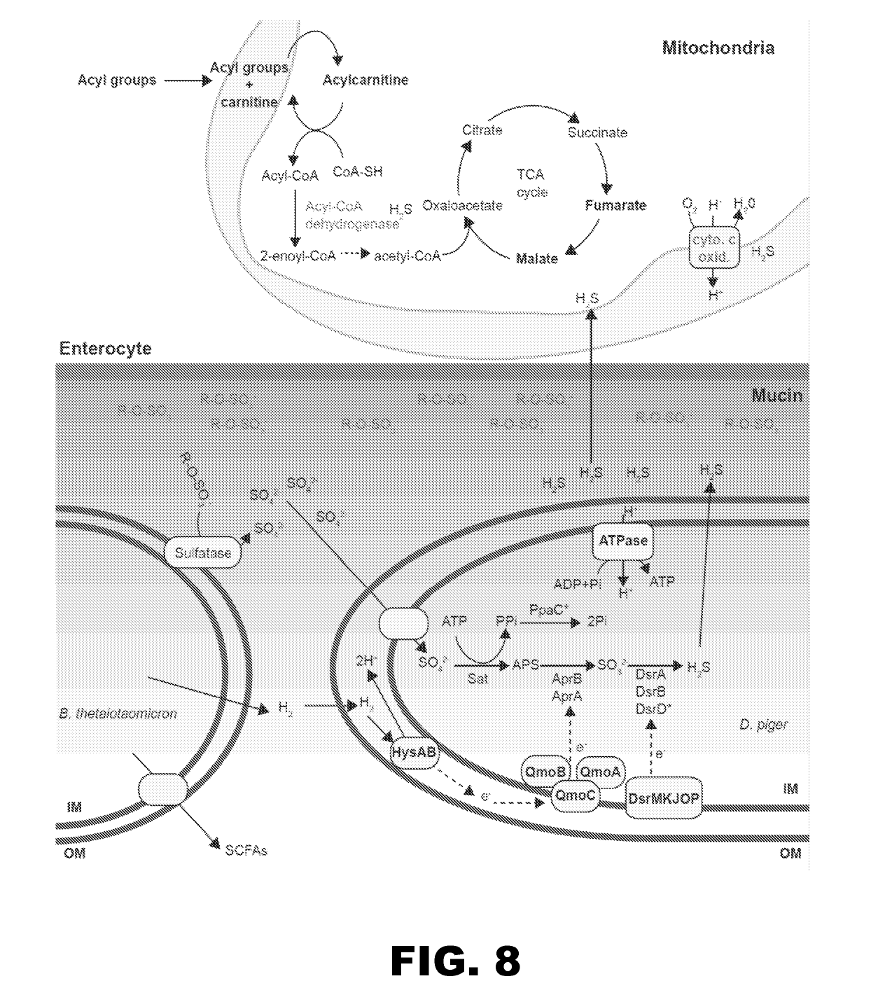

Taken together, the metabolic profiling and microbial and mouse RNA-Seq analyses suggest that high levels of H₂S generated by D. piger in the presence of dietary chondroitin sulfate result in lower host metabolic activity in the colon and less uptake of nutrients from luminal contents (FIG. 8). These results are consistent with a previous study that showed that daily colonic infusions of mM levels of H₂S significantly diminished the ex-vivo oxidative capacity of colonocytes (Moore et al., 1997). The net host effect of co-colonization with D. piger (i.e., increased microbial fermentative activity and decreased colonic oxidation of substrates) did not appear to translate into a significant difference in epididymal fat pad weight (mean±SEM: 30.3±2.3 (8-member) versus 23.6±1.8 (8-member plus D. piger) mg/g body weight, respectively; p=0.051).

The reported effects of H₂S on gut mucosal barrier function and immune activation in preclinical models have varied from promotion of inflammation to prevention of colitis (Pitcher et al., 2000; Levine et al., 1998; Wallace et al., 2009). Moreover, a severe decrease in oxidative metabolism in the colonic mucosa of rats results in inflammation (Roediger and Nance, 1986). In these studies, applying mouse RNA-Seq to the proximal colon revealed that colonization with D. piger was associated with significantly lower levels of mRNA encoding the tight junction protein claudin-4 plus higher levels of matrix metalloproteinase-7 (p<0.005, fold change >2 or <-2; Table 5). Histological inspection of the distal colon tissue did not show evidence of an ongoing inflammatory process in either group of mice consuming the HF/HS diet, Thus, deliberately increasing D. piger and H₂S levels with chondroitn sulfate did not have detectable effects on these measures of gut barrier integrity.

### Example 8

### Prospectus

To improve health status through personalized nutritional recommendations, the characteristics of a given diet, including its fermentable substrates, bioactive compounds and energy content, should be matched not only to the genetic makeup of the individual, but also to the metabolic potential of their intestinal microbiota. Developing conceptual and pragmatic strategies for manipulating the proportional representation and metabolic activities of gut microbes occupying different trophic positions in food webs, and identifying genetic and metabolic biomarkers of their niches and of the effects of such manipulations, requires preclinical models. These models should be representative of the human gut microbiota, yet with a sufficient degree of definition, simplification, and ease of manipulation, so that rules governing the operations of the microbiota can be deciphered through comprehensive characterization of community dynamics, microbial-host co-metabolism, and host physiology. Importantly, systems are needed where proof-of-concept therapeutic tests can be conducted through deliberate addition or subtraction of microbes and components of the diet and the effects on host physiology deciphered. Gnotobiotic mice experiments of the type described in the present report, where the effects of altering the hydrogen economy of a model human gut microbiota through (i) deliberate manipulation of the representation of a common human gut hydrogenotroph and a common component of human diets, (ii) inactivation of genes involved in key metabolic pathways within that hydrogenotroph and in a community partner with whom it shares food, and (iii) collection of datasets of different types (DNA-, mRNA- and metabolic-level) under highly controlled and replicated conditions, should be helpful in this regard.

This study focused on a sulfate-reducing bacterium because of its ability to generate H₂S and its possible relationship to human health (Babidge et al., 1998; Levine et al., 1998; Moore et al., 1997; Loubinoux et al., 2002a). There is great interpersonal variation among humans for carriage of SRB (Stewart et al., 2006; Christophersen et al., 2011; Hansen et al., 2011). The ability of the D. piger mutant library to invade an established community of moderate complexity suggests that this species could be introduced into humans lacking SRB to improve fermentation activity. Furthermore, levels of D. piger and H₂S could be altered by dietary components (e.g., chondroitin sulfate). An additional benefit of practical and societal importance is that these types of simplified, defined preclinical gnotobiotic animal models of the human gut microbiota provide an initial means to rigorously assess the impact of new foods, existing or new dietary supplements, flavor enhancers, food preservatives, or new approaches to food processing whose health effects or benefits are unclear.

### Methods for Examples 1-8

### Gnotobiotic Husbandry

All experiments involving mice were performed using protocols approved by the Washington University Animal Studies Committee. Mice belonging to the NRMI inbred strain were maintained in plastic flexible film gnotobiotic isolators under a strict 12 h light cycle (lights on at 0600) and fed diets ad libitum. Diets are listed in Table 7 and were sterilized by irradiation.

In Vitro Cross Feeding Between B. thetaiotaomicron and D. piger

Exponential phase cultures of B. thetaiotaomicron Δbt0238 and the isogenic wild-type parental strain (Benjdia et al., 2011; kindly provided by Eric Martens, University of Michigan and Olivier Berteau, INRA), grown in Mega Medium 2.0, were inoculated under anaerobic conditions (atmosphere of 5% H₂, 20% CO₂ and 75% N₂) into Balch tubes containing minimal medium supplemented with either 0.5% (w/v) chondroitin sulfate purified from shark cartilage (Sigma) or 0.5% fructose (Sigma) (n=6 tubes/carbon substrate/strain). Anaerobic cultures were incubated at 37° C. and growth was monitored at OD600 until cells reached late exponential phase (with the exception of B. thetaiotaomicron Δbt0238 which failed to grow in minimal medium plus chondroitin sulfate). Samples were taken and immediately frozen in liquid nitrogen for GC-MS analysis to provide the background levels of H₂S prior to D. piger growth. Cultures representing the same strain and carbon substrate were combined and bacteria were pelleted by centrifugation at 3,200×g at 4° C. for ˜20 min. The supernatant was removed and sterilized by passage through a 0.22 μm filter (Fisher). To allow for potential D. piger growth, we added lactate (to a final concentration of 30 mM), yeast extract (to 1 mg/mL), NH₄Cl (to 20 mM) and a mixture of vitamins and minerals (ATCC; 1× final concentration). The pH of the conditioned medium was adjusted to ˜7.0 using potassium phosphate buffer (pH 7.2). One half of each conditioned medium preparation was used to fill anaerobic Balch tubes (in triplicate) while sulfate (14 mM Na₂SO₄ and 4.1 mM MgSO₄) was added to the remaining conditioned medium prior to filling the tubes (in triplicate). A 100 μL aliquot of a late exponential phase culture of D. piger GOR1 (grown in SRB641 medium) was added to each tube containing the conditioned medium, and the tubes were incubated at 37° C. Samples were taken during exponential phase (OD600 0.28-0.44) for those cultures with growth and at this same time point for cultures without growth, and immediately frozen in liquid nitrogen for GC-MS analysis of H₂S levels.

Multiplex Pyrosequencing of Amplicons Generated from the aprA Gene

DNA was isolated from frozen fecal specimens obtained from healthy adults living in the USA who were recruited to a previously described and completed study using protocols approved by the Washington University HRPO (Turnbaugh et al., 2009; Hansen et al., 2011). An aliquot of fecal DNA was used for PCR amplification and sequencing of a conserved region of subunit A of the adenosine-5′-phosphosulfate reductase gene (aprA) present in sulfate-reducing bacteria using primers adapted from Deplancke et al. (2000). Amplicons (˜466 bp) were generated by using (i) modified primer AprA forward primer (5′-CCATCTCATCCCTGCGTGTCTCCGACTCAGNNNNNNNNNNTGGCAGATMATGATY MACGG-3′) (SEQ ID NO: 13) which consists of 454 FLX Titanium Amplicon primer A (underlined), a sample specific 10-mer barcode (N's) and the AprA primer (italics) and (ii) a modified AprA reverse primer (5′-CCTATCCCCTGTGTGCCTTGGCAGTCTCAG GGGCCGTAACCGTCCTTGAA (SEQ ID NO: 14) which contains 454 FLX titanium amplicon primer B (underlined), and the bacterial primer AprA (italics). Three replicate polymerase chain reactions were performed for each fecal DNA sample: each 20 μL reaction contained 50 ng of purified fecal DNA (Qiaquick, QIAGEN), 8□μL 2.5× HotMaster PCR Mix (Eppendorf), 0.25 μM of the forward primer and 0.1 μM of the reverse primer. PCR conditions consisted of an initial denaturation step performed at 95° C. for 4 min, followed by 35 cycles of denaturation (95° C. for 20 sec), annealing and amplification (65° C. for 1 min). Amplicons generated from each set of three reactions were subsequently pooled and purified using Ampure magnetic beads (Agencourt). The amount of purified DNA obtained was quantified using Picogreen (Invitrogen), and equimolar amounts of barcoded samples were pooled for each subsequent multiplex 454 FLX pyrosequencer run. aprA amplicon sequences were processed using the QIIME (v1.2) suite of software tools (Caporaso et al., 2010); fasta files and a mapping file indicating the sequence of the 10 nt barcode that corresponded to each sample were used as inputs.

COPRO-Seq

DNA isolated from feces (and cecal contents) was used to prepare libraries for shotgun Illumina sequencing (McNulty et al., 2011). Briefly, 1 μg of DNA from each sample was fragmented by sonication to an average size of ˜500 bp and subjected to enzymatic blunting and adenine tailing. Customized Illumina adapters containing maximally distant 4 bp or 8 bp barcodes were ligated to the A-tailed DNA. Barcoded libraries were then pooled, subjected to gel electrophoresis for size selection (˜200 bp) and the size-selected DNA amplified by PCR using primers and cycling conditions recommended by Illumina. Amplicons were purified (QIAquick PCR Purification Kit, Qiagen) and sequenced using an Illumina GA-IIx or HiSeq2000 instrument, with libraries loaded onto the flow cell at a concentration of 2.0-2.5 pM. A previously described custom software pipeline was used to process and analyze the resulting COPRO-Seq datasets (McNulty et al., 2011).

qPCR Measurements of D. piger Colonization

qPCR was performed by using an Mx3000P real-time PCR system (Stratagene). Reaction mixtures (25 μL) contained SYBR Green supermix (Bio-Rad), 400 nM D. piger-specific primers (see below), and 10 ng of DNA isolated from feces or cecal contents. Primer pairs targeted the 16S rRNA gene of D. piger (DpigGOR1_fwd (SEQ ID NO: 15) 5′-AAAGGAAGCACCGGCTAACT-3′, DpigGOR1_rev (SEQ ID NO: 16) 5′-CGGATTCAAGTCGTGCAGTA-3′). Amplification conditions were 55° C. for 2 min and 95° C. for 15 min, followed by 40 cycles of 95° C. (30 sec), 55° C. (45 sec), and 72° C. (30 sec). Data were collected at 78° C., 80° C., 82° C., and 84° C. The amount of D. piger DNA from each genome in each PCR was calculated by comparison to a standard curve of genomic DNA prepared in the same manner from D. piger monocultures. Data were converted to genome equivalents (GE) by calculating the mass of D. piger genomic DNA/cell (˜3.4×10⁶ fg) and normalized by fecal weight.

Microbioal RNA-Seq

Fecal samples obtained from mice, and from bacteria cultured under various defined nutrient conditions were immediately frozen at −80° C. and maintained at this temperature prior to processing. All samples were treated with RNAProtect (Qiagen). Each frozen sample was suspended in a solution containing 500 μL of acid-washed glass beads (Sigma-Aldrich), 500 μL of extraction buffer A (200 mM NaCl, 20 mM EDTA), 210 μL of 20% SDS, and 500 μL of a mixture of phenol:chloroform:isoamyl alcohol (125:24:1, pH 4.5; Ambion), and lysed by using a bead beater (BioSpec Products; maximal setting; 4 min at room temperature). Cellular debris was removed by centrifugation (8,000×g; 3 min at 4° C.). The extraction was repeated, and nucleic acids were precipitated with isopropanol and sodium acetate (pH 5.5). Details about protocols used for removing residual DNA from RNA preparations, rRNA depletion, double-stranded cDNA synthesis, and multiplex sequencing with the Illumina Hi-Seq instrument, as well as our data analysis pipeline have been described previously (Faith et al., 2011; Rey et al., 2010).

RNA-Seq Analysis of Proximal Colon Samples

Transcriptional profiling of mouse samples was performed as previously described (Marioni J C, 2008). Frozen proximal colon tissue was homogenized in 1 mL of Trizol (Invitrogen) and total RNA was purified using the Qiagen RNeasy mini kit and two DNAse treatments including one on column DNase treatment (Qiagen) followed by the Zymo DNA-Free RNA kit (Zymo Research). mRNA was further purified using Dynabeads mRNA Purification Kit (Invitrogen), reverse-transcribed to ds cDNA and Illumina libraries were generated using the NEBNext mRNA Sample Prep Reagent Set 1 (NEB) following the manufacturer's protocol. In-house barcoded DNA adaptors were ligated to cDNA to allow multiplexing of 7 libraries per lane on the Illumina HiSeq 2000 (Illumina).

Construction of D. piger Transposon Mutagenesis Vector

To generate the D. piger GOR1 transposon mutant library, we modified the original INSeq vector, pSAM_Bt (Goodman et al., 2009), by (i) switching the transposon's ermG antibiotic resistance gene with one known to work in Desulfovibrio vulgaris [aadA (spectinomycin resistance)], (ii) using the promoter region from a highly expressed D. piger gene to drive expression of the mariner transposase, and (iii) optimizing codon usage for the transposase based on the D. piger genome. This effort involved the following procedures. aadA was PCR amplified from pMO719 (Keller et al., 2009; kindly provided by Judy Wall, University of Missouri) using primers Mfel aadA (SEQ ID NO: 17) 5′ (5′-GGGAATTCCAATTGAGACCAGCCAGGACAGAAATGCC) and Xbal (SEQ ID NO: 18) aadA 3′ (5′-CTAGTCTAGACGGGGTCTGACGCTCAGTGGAACG). The resulting PCR fragment was digested with Mfel and Xbal, and ligated into pSAM_Bt (Goodman et al., 2009) after excision of its ermG gene with Mfel and Xbal, creating pSAM-aadA. The mariner transposase gene was synthesized (GenScript) using codon sequences optimized to the D. piger GOR1 genome, and a 1,052 bp fragment containing this gene was excised with Ndel and Notl from the pUC57 vector into which it had been originally cloned. The D. piger codon-optimized mariner transposase was then ligated to the linearized pSAM-aadA, creating pSAM-aadA*. Finally, we recovered the 5′ proximal region of a highly expressed D. piger gene (DpigGOR12316) that encodes the a subunit of sulfite reductase using PCR primers BamHIDpig23165′ (SEQ ID NO: 19) (5′-ACGCGGATCCGGGCGCTCCCGCAGGGGACAGCGG) and Dpig2316prom3 (SEQ ID NO: 20) (5′-GCCATACCTCCACATGGTTTGTTGTATCAC) and D. piger GOR1 genomic DNA. The resulting amplicon was (i) digested with BamHI and (ii) ligated into pSAM-aadA*, which had been initially cut with Ndel and blunt ended by filling in the 5′ overhang using T4 DNA polymerase and then digested with BamHI, yielding pSAM-aadA*-2316. Throughout the cloning process, we confirmed the correct DNA sequence for each construct by DNA sequencing.

Transposon Mutagenesis of D. piger GOR1

We used the following procedure to mutagenize D. piger GOR1 via anaerobic conjugation with a diaminopimelic acid (DAP) auxotrophic strain of E. coli, β2163(Demarre et al., 2005), harboring pSAM-aadA*-2316. Aliquots (1.25 OD600 units) of exponential phase cultures of D. piger GOR1, grown anaerobically at 37° C. in SRB641 medium (see Table 7), and the E. coli mating strain (β2163/pSAM-aadA*-2316), grown aerobically at 37° C. in LB medium containing 100 μg ampicillin/mL and 300 μg diaminopimelic acid (DAP)/mL, were combined on a filter that was then transferred to MegaMedium 2.0 (see Table 7) containing DAP (300 μg/mL) and dithiothreitol (0.5 g/L) in lieu of cysteine as the reductant (the oxidized form of cysteine, cystine, competes with DAP for cellular uptake and can inhibit growth of the DAP auxotrophic strain; Berger and Heppel, 1972). We incubated the filter matings overnight at 37° C. under strictly anaerobic conditions (atmosphere of 5% H₂, 20% CO₂, and 75% N₂), and then resuspended the cells in 2.5 mL of MegaMedium 2.0. To obtain isolated D. piger transconjugants, we diluted the cell suspension 1:3 in MegaMedium 2.0 and plated 300 μL aliquots onto large Petri dish plates (150×15 mm, Falcon) containing MegaMedium 2.0/agar supplemented with spectinomycin (300 μg/mL). These plates lacked DAP and contained cysteine instead of DTT to counterselect against growth of the E. coli donor strain. Plates were incubated at 37° C. under strictly anaerobic conditions for 2 days to allow spectinomycin-resistant transconjugants of D. piger GOR1 to grow. Colonies (˜40,000) were scraped from plates and pooled together in MegaMedium 2.0 with 20% glycerol and frozen at −80° C. in 0.5 mL aliquots (in cryovials).

In Vitro INSeq Analysis of the D. piger Mutant Library

A 0.5 mL aliquot of the D. piger transposon mutant library was diluted in SRB Base medium (Table 7) to an OD600 of ˜6 under anaerobic conditions, and 0.5 mL aliquots of this dilution were the introduced into duplicate flasks containing 500 mL of SRB medium (see next paragraph). The resulting culture was incubated at 37° C. under anaerobic conditions to late exponential phase (OD600˜0.5). Aliquots (2 mL) were then inoculated into duplicate flasks of containing 500 mL of fresh SRB medium. Growth of this second set of flasks was monitored and samples were harvested during the late exponential phase of growth (OD600˜0.5) for INSeq analysis.

We used SRB 20 amino acid medium (Table 7), or the SRB Base medium (Table 7) with both yeast extract and NH₄Cl, supplemented with (i) pyruvate alone (60 mM final concentration) or (ii) pyruvate (60 mM final concentration) and sulfate (14 mM Na₂SO₄, □4.1 mM MgSO₄) or (iii) lactate (30 mM) and sulfate (14 mM Na₂SO₄, 4.1 mM MgSO₄), or (iv) formate (60 mM), acetate (10 mM) and sulfate (14 mM Na₂SO₄, 4.1 mM MgSO₄), or (v) acetate (10 mM) and sulfate (14 mM Na₂SO₄, 4.1 mM MgSO₄). The last condition was used for testing H₂ as the electron donor and done in 125 mL serum bottles filled with 50 mL of medium and incubated with a headspace of 80% H₂/20% CO₂ (30 psi of pressure) at 37° C.

INSeq Library Preparation

INSeq analysis involves the following steps (i) isolation and purification of DNA; (ii) linear PCR enrichment of the transposon/chromosomal junction; (iii) purification and double-strand synthesis of the PCR product; (iv) digestion with restriction enzymes for DNA size selection, (v) barcode ligation, (vi) PCR amplification and (vii) Illumina DNA sequencing. We followed the DNA preparation and INSeq protocol as previously described (Goodman et al., 2011) with the following exceptions. Linear PCR was done with 2× Pfx buffer (20 μL/100 μL PCR reaction) and the linear PCR was run on a thermocycler using the following conditions: 94□C. for 2 min, followed by 50 cycles of 94° C. for 15 sec, 60° C. for 30 sec, and 68° C. for 30 sec. The final PCR amplification was run on a thermocycler at 94° C. for 2 min, followed by 20 cycles of 94° C. for 15 sec, 55° C. for 1 min, 68° C. for 30 sec and then 68° C. for 4 min. Amplicons were sequenced using an Illumina HiSeq instrument. Sequencing data was analyzed using the DESeq package (Anders and Huber, 2010).

Identification of Essential Genes

We identified a list of D. piger genes likely to be essential through the following method: we assembled the read counts at each TA site from the input libraries of five independent library preparations and sequencing runs (each insertion site needed more than 3 reads to be counted as an insertion). Additionally, only insertions located within the first 80% of the coding region (relative to the 5′ end) were considered, since those would likely disrupt gene function. From this data we compiled a list of putatively essential genes based on matching two criteria: (i) there were no insertions located within the 80% proximal region of the gene, and (ii) the gene has a significant probability of having a transposon insertion (p-value <0.05). The probability that a given gene with n TA sites has k insertions follows a binominal distribution with a success probability θ, in which θ was conservatively estimated to be the fraction of TA sites containing insertions in the entire genome. To assess the statistical significance of the observed gene without disrupted insertions, the p-value was calculated as

$$P ( k ; n , θ ) = ( \begin{matrix}n \\ k\end{matrix} ) {{θ}^{k} ( 1 - θ )}^{( n - k )} .$$

Gas Chromatography-Mass Spectroscopy: Targeted GC-MS of Short Chain Fatty Acid Measurements—

Cecal contents or fecal pellets were weighed in 4 mL polytetrafluoroethylene (PTFE) screw cap vials and 10 μL of a mixture of internal standards (20 mM of acetic acid-¹³C₂, D₄, propionic acid-D₆, butyric acid-¹³C₄, lactic acid-3,3,3-D₃ and succinic acid-¹³C₄) was subsequently added to each vial, followed by 20 μL of 33% HCl and 1 mL diethyl ether. The mixture was vortexed vigorously for 10 min and then centrifuged (4,000×g, 5 min). The upper organic layer was transferred to another vial and a second diethyl ether extraction was performed. After combining the two ether extracts, a 60 μL aliquot was removed, combined with 20 μL N-tert-butyldimethylsilyl-N-methyltrifluoroacetamide (MTBSTFA) in a GC auto-sampler vial with a 200 μL glass insert, and incubated for 2 h at room temperature.

Samples were analyzed in a randomized order. Derivatized samples (1 μL) were injected with 15:1 split into an Agilent 7890A gas chromatography system coupled with 5975C mass spectrometer detector (Agilent, CA). Analyses were carried on a HP-5MS capillary column (30 m×0.25 mm, 0.25 μm film thickness, Agilent J & W Scientific, Folsom, Calif.) using electronic impact (70 eV) as ionization mode. Helium was used as a carrier gas at a constant flow rate of 1.26 mL/min and the solvent delay time was set to 3.5 min. The column head-pressure was 10 p.s.i. The temperatures of injector, transfer line, and quadrupole were 270° C., 280° C. and 150° C., respectively. The GC oven was programmed as follows: 45° C. held for 225 min; increased to 200° C. at a rate of 20° C./min; increased to 300° C. at a rate of 100° C./min; and finally held for 3 min.

Quantification of SCFA was performed by isotope dilution GC-MS using selected ion monitoring (SIM). For SIM analysis, the m/z for native and labeled molecular peaks for SCFA quantified were 117 and 122 (acetate), 131 and 136 (propionate), 145 and 149 (butyrate), 261 and 264 (lactate) and 289 and 293 (succinate), respectively. Various concentrations of standards were spiked into control samples to prepare the calibration curves for quantification.

Targeted GC-MS of Hydrogen Sulfide—

Sample preparation was based on a previously described procedure (Hyspler et al., 2002) with some modifications. Frozen cecal contents were cut on dry ice into 10 mg aliquots and weighed in 2 mL screw cap vials. 150 μL of 5 mM benzalkonium chloride in oxygen-free water, saturated with sodium tetraborate, was added to each vial, followed by 400 μL of 20 mM of pentafluorobenzylbromide (PFBBr) in toluene and 100 μL of ethyl acetate containing 15 □μM 4-chloro-benzyl methyl sulfide (internal standard). Vials were closed tightly with a PTFE-coated cap and the mixture was shaken in a 55.8° C. oven for 4 h. A saturated solution of potassium dihydrogenphosphate (in water) was added (200 μL) and the mixture was vigorously vortexed for 1 min. The organic and inorganic layers were separated by centrifugation (3,220×g for 10 min at 4° C.).

Samples were analyzed in a randomized order. Samples (1 μL) were injected without split into an Agilent 7890A gas chromatography system coupled with 5975C mass spectrometer detector. Analyses were carried on a HP-5MS capillary column (see above) using electronic impact (70 eV) as ionization mode. Helium was used as a carrier gas at a constant flow rate of 1.1 mL/min and the solvent delay time was set to 5.5 min. The column head-pressure was 8.23 p.s.i. The temperatures of the injector, transfer line, and quadrupole were 250° C., 280° C. and 150° C., respectively. The GC oven was programmed as follows: 100° C. held for 1 min; increased to 250° C. at a rate of 8° C./min, increased to 300° C. at a rate of 50° C./min; and finally held for 3 min.

Non-Targeted GC-MS Analysis—

Cecal contents or fecal pellets were weighed and 20 volumes of HPLC grade water were added. Homogenization was performed using a bead beater (Biospec Products) without beads for 2 min. After centrifugation (20,800×g for 10 min at 4° C.), a 200 μL aliquot of the supernatant was transferred to a clean tube. Ice-cold methanol (400 μL) was added to each sample; the mixture was vortexed, and subsequently centrifuged at 20,800×g for 10 min at 4° C. A 500 μL aliquot of the resulting supernatant together with 10 μL of lysine-¹³C₆,¹⁵N₂ (2 mM) was evaporated to dryness using a speed vacuum. Derivatization of all dried supernatants followed a method adapted with modifications from Gao et al. (2010). Briefly, 80 μL of a solution of methoxylamine (15 mg/mL in pyridine) was added to methoximate reactive carbonyls (incubation for 16 h for 37° C.), followed by replacement of exchangeable protons with trimethylsilyl groups using N-methyl-N-(trimethylsilyl) trifluoroacetamide (MSTFA) with a 1% v/v catalytic admixture of trimethylchlorosilane (Thermo-Fisher Scientific, Rockford, Ill.) (incubation at 70° C. for 1 h). Finally, 160 μL heptane was added to the derivatives prior to injection.

A 1 μL aliquot of each derivatized sample was injected without split into the GC-MS system described above. Analyses were carried on a HP-5MS capillary column (see above) using electronic impact (70 eV) as ionization mode. Helium was used as a carrier gas at a constant flow rate of 1 mL/min; the solvent delay time was set to 5.5 min. The column head-pressure was 8.23 p.s.i. Temperatures of the injector, transfer line, and source were 250° C., 290° C. and 230° C., respectively. The GC oven was programmed as follows: 60° C. held for 2 min; increased to 140° C. at a rate of 10° C./min; increased to 240° C. at a rate of 4° C./min; increased to 300° C. at a rate of 10° C./min; and finally held at 300° C. for 8 min. Metabolite identification was done by co-characterization of standards.

Data in instrument specific format (.D) were converted to common data format (.cdf) files using MSD ChemStation (E02.01, Agilent, CA); the .cdf files were extracted using Bioinformatics Toolbox in the MATLAB 7.1 (The MathWorks, Inc., Natick, Mass.), along with custom scripts (Cheng et al., 2011) for alignment of data in the time domain, automatic integration, and extraction of peak intensities. The resulting three dimension data set included sample information, peak retention time and peak intensities. Data were then mean centered and unit variance scaled for multivariate analysis.

Quality Control of Metabolomics Data—

Pooled quality control (QC) samples were prepared from 20 μL of each sample and analyzed together with the other samples. The QC samples were also inserted and analyzed in every 10 samples. To exclude false positives, the raw data of statistical significant metabolites were re-evaluated in MSD ChemStation E.02.01.1177 (Agilent, CA).

Ultra High Performance Liquid Chromatography-Mass Spectrometry (UPLC-MS)

Frozen cecal samples were combined with 20 volumes of cold methanol, one volume of cysteine ¹³C₆,¹⁵N₂ (4 mM) and mixed for 2 min in a bead beater (Biospec Products; maximal setting; no beads added). Samples were then incubated at −20° C. for 1 h, and subsequently centrifuged 10 min at 20,800×g. The supernatant (300 μL) was collected and dried in a SpeedVac at room temperature. Dried samples were resuspended in 100 μL of 95:5 water:ethanol, clarified for 5 min by centrifugation at 20,800×g for 10 min at 4° C., and the supernatant vias separated for UPLC-MS. Analyses were performed on a Waters Acquity I Class UPLC system (Waters Corp., Milford, Mass.) coupled to an LTQ-Orbitrap Discovery (Thermo Fisher Corporation). A 5 μL injection volume and flow rate of 0.3 mL/min were used for both the Discovery HS F5 PFPP column (150 mm×2.1 mm, 3 μm particle size; Sigma-Aldrich) and the 150 mm×2.1 mm Waters BEH C18 1.7 μm particle column. Mobile phases for positive ion mode were (A) 0.1% formic acid in water and (B) 0.1% formic acid in acetonitrile, whereas negative ion mode used (A) 5 mM ammonium bicarbonate in water and (B) 5 mM ammonium bicarbonate in 95/5 acetonitrile/water. The capillary column was maintained at 325° C. with a sheath gas flow of 40 (arbitrary units), an aux gas flow of 5 (arbitrary units) and a sweep gas flow of 3 (arbitrary units), for both positive and negative injections. The spray voltage for the positive ion injection was 4.5 kV, and 4 kV for the negative ion injection.

### Ammonia Measurements

Ammonia levels in feces and cecal contents were quantified using an assay kit from Abcam (ab83360) and the protocol described by the manufacturer.

### REFERENCES FOR EXAMPLES 1-8

Anders, S. and Huber, W. (2010). Differential expression analysis for sequence count data. Genome Biol. 11, R106.
Ardawi, M. S., and Newsholme, E. A. (1985). Fuel utilization in colonocytes of the rat. Biochem. J. 231, 713-719.
Auchere, F. et al. (2006). Kinetics studies of the superoxide-mediated electron transfer reactions between rubredoxin-type proteins and superoxide reductases. J. Biol. Inorganic Chem. 11, 433-444.
Babidge, W. et al. (1998). Sulfides impair short chain fatty acid beta-oxidation at acyl-CoA dehydrogenase level in colonocytes: implications for ulcerative colitis. Mol. Cell. Biochem. 181, 117-124.
Barthe, L. et al. (2004). In vitro intestinal degradation and absorption of chondroitin sulfate, a glycosaminoglycan drug. Arzneimittel-Forschung 54, 286-292.
Benjdia, A. et al. (2011). Sulfatases and a radical S-adenosyl-L-methionine (AdoMet) enzyme are key for mucosal foraging and fitness of the prominent human gut symbiont, Bacteroides thetaiotaomicron. J. Biol. Chem. 286, 25973-25982.
Berger, E. A., and Heppel, L. A. (1972). A binding protein involved in the transport of cystine and diaminopimelic acid in Escherichia coli. J. Biol. Chem. 247, 7684-7694.
Bergman, E. N. (1990). Energy contributions of volatile fatty acids from the gastrointestinal tract in various species. Physiol. Rev. 70, 567-590.
Bhala, A. et al. (1995). Clinical and biochemical characterization of short-chain acyl-coenzyme A dehydrogenase deficiency. J. Pediatrics 126, 910-915.
Blachier, F. et al. (2009). Metabolism and functions of L-glutamate in the epithelial cells of the small and large intestines. Am. J. Clin. Nutr. 90, 814S-821 S.
Caffrey, S. M. et al. (2007). Function of periplasmic hydrogenases in the sulfate-reducing bacterium Desulfovibrio vulgaris Hildenborough. J. Bacteriol. 189, 6159-6167.
Cantarel, B. L. et al. (2009). The Carbohydrate-Active EnZymes database (CAZy): an expert resource for Glycogenomics. Nucleic Acids Res. 37, D233-238.
Caporaso, J. G. et al. (2010). QIIME allows analysis of high-throughput community sequencing data. Nat. Methods 7, 335-336.
Cheng, J., Yuan, C., and Graham, T. L. (2011). Potential defense-related prenylated isoflavones in lactofen-induced soybean. Phytochemistry 9, 875-881.
Christophersen, C. T. et al. (2011). Overestimation of the abundance of sulfate-reducing bacteria in human feces by quantitative PCR targeting the Desulfovibrio 16S rRNA gene. Appl. Environ. Microbiol. 77, 3544-3546.
Cordruwisch, R. et al. (1988). The Capacity of Hydrogenotrophic Anaerobic-Bacteria to Compete for Traces of Hydrogen Depends on the Redox Potential of the Terminal Electron-Acceptor. Arch. Microbiol. 149, 350-357.
Curno, R. et al. (2008). Studies of a urinary biomarker of dietary inorganic sulphur in subjects on diets containing 1-38 mmol sulphur/day and of the half-life of ingested 34SO42-. Eur. J. Clin. Nutr. 62, 1106-1115.
Demarre, G. et al. (2005). A new family of mobilizable suicide plasmids based on broad host range R388 plasmid (IncW) and RP4 plasmid (IncPalpha) conjugative machineries and their cognate Escherichia coli host strains. Research in Microbiol. 156, 245-255.
Deplancke, B. et al. (2000). Molecular ecological analysis of the succession and diversity of sulfate-reducing bacteria in the mouse gastrointestinal tract. Appl. Environ. Microbiol. 66, 2166-2174.
Devkota, S. et al. (2012). Dietary-fat-induced taurocholic acid promotes pathobiont expansion and colitis in II10(−/−) mice. Nature 487, 104-108.
Dolla, A. et al. (2000). Deletion of the hmc operon of Desulfovibrio vulgaris subsp. vulgaris Hildenborough hampers hydrogen metabolism and low-redox-potential niche establishment. Arch. Microbiol. 174, 143-151.
Faith, J. J. et al. (2011). Predicting a human gut microbiota's response to diet in gnotobiotic mice. Science 333, 101-104.
Firmansyah, A. et al. (1989). Isolated colonocyte metabolism of glucose, glutamine, n-butyrate, and beta-hydroxybutyrate in malnutrition. Gastroenterology 97, 622-629.
Fischbach, M. A., and Sonnenburg, J. L. (2011). Eating for two: how metabolism establishes interspecies interactions in the gut. Cell Host & Microbe 10, 336-347.
Fite, A. et al. (2004). Identification and quantitation of mucosal and faecal desulfovibrios using real time polymerase chain reaction. Gut 53, 523-529.
Flint, H. J. et al. (2008). Polysaccharide utilization by gut bacteria: potential for new insights from genomic analysis. Nature Rev. Microbiol. 6, 121-131.
Gao, X. et al. (2010). Development of a quantitative metabolomic approach to study clinical human fecal water metabolome based on trimethylsilylation derivatization and GC/MS analysis. Analytical Chem. 82, 6447-6456.
Gibson, G. R. et al. (1988). Use of a three-stage continuous culture system to study the effect of mucin on dissimilatory sulfate reduction and methanogenesis by mixed populations of human gut bacteria. Appl. Environ. Microbiol. 54, 2750-2755.
Gomes, C. M. et al. (1997). Studies on the redox centers of the terminal oxidase from Desulfovibrio gigas and evidence for its interaction with rubredoxin. J. Biol. Chem. 272, 22502-22508.
Goodman, A. L. et al. (2009). Identifying genetic determinants needed to establish a human gut symbiont in its habitat. Cell Host & Microbe 6, 279-289.
Goodman, A. L. et al. (2011). Identifying microbial fitness determinants by insertion sequencing using genome-wide transposon mutant libraries. Nat. Protoc. 6, 1969-1980.
Hansen, E. E. et al. (2011). Pan-genome of the dominant human gut-associated archaeon, Methanobrevibacter smithii, studied in twins. Proc. Natl. Acad. Sci. USA 108, 4599-4606.
HMP consortium (2012). Structure, function and diversity of the healthy human microbiome. Nature 486, 207-214.
Hyspler, R. et al. (2002). A simple, optimized method for the determination of sulphide in whole blood by GC-MS as a marker of bowel fermentation processes. J. Chromatography B, Analytical Technol. Biomedical & Life Sciences 770, 255-259.
Karlsson, C. L. et al. (2012). The microbiota of the gut in preschool children with normal and excessive body weight. Obesity 20, 2257-2261.
Keller, K. L. et al. (2009). Development of a markerless genetic exchange system for Desulfovibrio vulgaris Hildenborough and its use in generating a strain with increased transformation efficiency. Applied Environ. Microbiol. 75, 7682-7691.
Keon, R. G. et al. (1997). Deletion of two downstream genes alters expression of the hmc operon of Desulfovibrio vulgaris subsp. vulgaris Hildenborough. Arch. Microbiol. 167, 376-383.
Kimura, R. E., and Mich, J. Z. (1991). The oxidation of 3-hydroxybutyrate in developing rat jejunum. J. Pediatric Gastroenterol. & Nutr. 13, 347-353.
Kler, R. S. et al. (1991). Quantitation of acyl-CoA and acylcarnitine esters accumulated during abnormal mitochondrial fatty acid oxidation. J. Biol. Chem. 266, 22932-22938.
Levine, J. et al. (1998). Fecal hydrogen sulfide production in ulcerative colitis. Am. J. Gastroenterol. 93, 83-87.
Loubinoux, J. et al. (2002a). Sulfate-reducing bacteria in human feces and their association with inflammatory bowel diseases. FEMS Microbiol. Ecol. 40, 107-112.
Loubinoux, J. et al. (2002b). Reclassification of the only species of the genus Desulfomonas, Desulfomonas pigra, as Desulfovibrio piger comb. nov. Int. J. Syst. Evol. Microbiol. 52, 1305-1308.
Lumppio, H. L. Shenvi et al. (2001). Rubrerythrin and rubredoxin oxidoreductase in Desulfovibrio vulgaris: a novel oxidative stress protection system. J. Bacteriol. 183, 101-108.
Macfarlane, S., and Macfarlane, G. T. (2003). Regulation of short-chain fatty acid production. Proc. Nutr. Soc. 62, 67-72.
Marioni J C et al. (2008). RNA-seq: an assessment of technical reproducibility and comparison with gene expression arrays. Genome Res. 18, 1509-17.
McNulty, N. P. et al. (2011). The impact of a consortium of fermented milk strains on the gut microbiome of gnotobiotic mice and monozygotic twins. Science Translational Med. 3, 106ra106.
Moore, J. W. et al. (1997). Hydrogen sulphide produces diminished fatty acid oxidation in the rat colon in vivo: implications for ulcerative colitis. Australian & New Zealand J. Surgery 67, 245-249.
Nava, G. M. et al. (2012). Abundance and diversity of mucosa-associated hydrogenotrophic microbes in the healthy human colon. ISME J. 6, 57-70.
Nicholls, P. (1975). The effect of sulphide on cytochrome aa3. Isosteric and allosteric shifts of the reduced alpha-peak. Biochim. Biophys. Acta 396, 24-35.
Pitcher, M. C. et al. (2000). The contribution of sulphate reducing bacteria and 5-aminosalicylic acid to faecal sulphide in patients with ulcerative colitis. Gut 46, 64-72.
Qin, J. et al. (2010). A human gut microbial gene catalogue established by metagenomic sequencing. Nature 464, 59-65.
Rawlings, N. D. et al. (2012). MEROPS: the database of proteolytic enzymes, their substrates and inhibitors. Nucleic Acids Res 40, D343-350.
Rey, F. E. et al. (2010). Dissecting the in vivo metabolic potential of two human gut acetogens. J. Biol. Chem. 285, 22082-22090.
Roediger, W. E., and Nance, S. (1986). Metabolic induction of experimental ulcerative colitis by inhibition of fatty acid oxidation. British J. Exp. Pathol. 67, 773-782.
Salyers, A. A., and O'Brien, M. (1980). Cellular location of enzymes involved in chondroitin sulfate breakdown by Bacteroides thetaiotaomicron. J. Bacteriol. 143, 772-780.
Scanlan, P. D. et al. (2009). Culture-independent analysis of desulfovibrios in the human distal colon of healthy, colorectal cancer and polypectomized individuals. FEMS Microbiol. Ecol. 69, 213-221.
Stams, A. J. (1994). Metabolic interactions between anaerobic bacteria in methanogenic environments. Antonie van Leeuwenhoek 66, 271-294.
Stams, A. J., and Plugge, C. M. (2009). Electron transfer in syntrophic communities of anaerobic bacteria and archaea. Nature Rev. Microbol. 7, 568-577.
Stewart, J. A. et al. (2006). Carriage, quantification, and predominance of methanogens and sulfate-reducing bacteria in faecal samples. Letters Applied Microbiol. 43, 58-63.
Truong, D. H. et al. (2006). Molecular mechanisms of hydrogen sulfide toxicity. Drug Metabol. Rev. 38, 733-744.
Turnbaugh, P. J. et al. (2009). A core gut microbiome in obese and lean twins. Nature 457, 480-484.
Voordouw, J. K., and Voordouw, G. (1998). Deletion of the rbo gene increases the oxygen sensitivity of the sulfate-reducing bacterium Desulfovibrio vulgaris Hildenborough. Applied Environ. Microbiol. 64, 2882-2887.
Wallace, J. L et al. (2009). Endogenous and exogenous hydrogen sulfide promotes resolution of colitis in rats. Gastroenterology 137, 569-578.
Wildschut, J. D. et al. (2006). Rubredoxin:oxygen oxidoreductase enhances survival of Desulfovibrio vulgaris hildenborough under microaerophilic conditions. J. Bacteriol. 188, 6253-6260.
Willis, C. L. et al. (1996). In vitro effects of mucin fermentation on the growth of human colonic sulphate-reducing bacteria. Anaerobe 2, 117-122.
Willis, C. L. et al. (1997). Nutritional aspects of dissimilatory sulfate reduction in the human large intestine. Current Microbiol. 35, 294-298.
Wolin, M. J., and Miller, T. L. (1983). Interactions of microbial populations in cellulose fermentation. Fed. Proc. 42, 109-113.
Yatsunenko, T. et al. (2012). Human gut microbiome viewed across age and geography. Nature 486, 222-227.
Zane, G. M., et al. (2010). Effect of the deletion of qmoABC and the promoter-distal gene encoding a hypothetical protein on sulfate reduction in Desulfovibrio vulgaris Hildenborough. Applied Environ. Microbiol. 76, 5500-5509.
Zhang, J., et al. (1998). Energy metabolism of rat colonocytes changes during the tumorigenic process and is dependent on diet and carcinogen. J. Nutr. 128, 1262-1269.
Zinkevich, V. V., and Beech, I. B. (2000). Screening of sulfate-reducing bacteria in colonoscopy samples from healthy and colitic human gut mucosa. FEMS Microbiol. Ecol. 34, 147-155.

1. A method for increasing microbial fermentative activity in the gut of a subject in need thereof, the method comprising administering a combination comprising a sulfated polysaccharide and an effective amount of at least one isolated Desulfovibrio species, wherein the at least one isolated Desulfovibrio species comprises at least one nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

2. A method for increasing the nutritional value of a diet, the method comprising administering to a subject as part of a diet a combination comprising a sulfated polysaccharide and an effective amount of at least one isolated Desulfovibrio species, wherein the at least one isolated Desulfovibrio species comprises at least one nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12), wherein the combination increases microbial fermentative activity in the gut of the subject, thereby increasing the nutritional value of the diet.

3. The method of claim 1, wherein the isolated Desulfovibrio species comprises at least 3 nucleic acids with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

4. The method of claim 1, wherein the isolated Desulfovibrio species comprises at least 6 nucleic acids with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

5. The method of claim 1, wherein the isolated Desulfovibrio species comprises at least 9 nucleic acids with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

6. The method of claim 1, wherein the isolated Desulfovibrio species comprises a nucleic acid with at least 80% identity to each nucleic acid in the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

7. The method of claim 1, wherein the identity is at least 90%.

8. The method of claim 1, wherein the identity is at least 94%.

9. The method of claim 1, wherein the sulfated polysaccharide is selected from the group consisting of a pentosan polysulfate, a fucoidan, a carrageenan, a sulfated glycosaminoglycan, and derivatives thereof.

10. The method of claim 1, wherein the combination further comprises an effective amount of at least one additional bacterial species selected from the group consisting of a saccharolytic bacterial species, a butyrate-producing bacterial species, and a combination thereof.

11. The method of claim 1, wherein at least one isolated Desulfovibrio species is Desulfovibrio piger and the sulfated polysaccharide is chondroitin sulfate.

12. The method of claim 1, wherein the method further comprises confirming the increase in microbial fermentative activity, wherein the measurement for increased microbial fermentative activity is selected from the group consisting of increased short chain fatty acids, increased hydrogen sulfide, increased abundance of the Desulfovibrio species, and combinations thereof.

13. A combination comprising a sulfated polysaccharide and an effective amount of an isolated Desulfovibrio species, wherein the at least one isolated Desulfovibrio species comprises at least one nucleic acid with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

14. (canceled)

15. (canceled)

16. The combination of any of claims 13, wherein the isolated Desulfovibrio species comprises 9 or more nucleic acids with at least 80% identity to a nucleic acid selected from the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

17. The combination of claim 13, wherein the isolated Desulfovibrio species comprises a nucleic acid with at least 80% identity to each nucleic acid in the group consisting of DpigGOR1_{—}1496 (SEQ ID NO: 1), DpigGOR1_{—}1497 (SEQ ID NO: 2), DpigGOR1_{—}0739 (SEQ ID NO: 3), DpigGOR1_{—}0740 (SEQ ID NO: 4), DpigGOR1_{—}1393 (SEQ ID NO: 5), DpigGOR1_{—}1398 (SEQ ID NO: 6), DpigGOR1_{—}0741 (SEQ ID NO: 7), DpigGOR1_{—}0744 (SEQ ID NO: 8), DpigGOR1_{—}0790 (SEQ ID NO: 9), DpigGOR1_{—}0792 (SEQ ID NO: 10), DpigGOR1_{—}0170 (SEQ ID NO: 11), and DpigGOR1_{—}0174 (SEQ ID NO: 12).

18. The combination of claim 13, wherein the identity is at least 90%.

19. The combination of claim 13, wherein the identity is at least 94%.

20. The combination of claim 13, wherein the sulfated polysaccharide is selected from the group consisting of a pentosan polysulfate, a fucoidan, a carrageenan, a sulfated glycosaminoglycan, and derivatives thereof.

21. The combination of claim 13, wherein the combination further comprises an effective amount of at least one additional bacterial species selected from the group consisting of a saccharolytic bacterial species, a butyrate-producing bacterial species, or a combination thereof.

22. The combination of any of claim 13, wherein the at least one isolated Desulfovibrio species is Desulfovibrio piger and the sulfated polysaccharide is chondroitin sulfate.
